(19) United States
(12) Patent Application Publication
(10) Pub. No.: US 2023/0276701 A1
(43) Pub. Date: Aug. 31, 2023

(54) ORGANIC COMPOUND AND ORGANIC LIGHT EMISSION DEVICE

(71) Applicant: OSAKA UNIVERSITY, Osaka (JP)

(72) Inventors: Daigo MIYAJIMA, Saitama (JP); Naoya AIZAWA, Saitama (JP); Yong-Jin PU, Saitama (JP); Atsuko NIHONYANAGI, Saitama (JP); Ryotaro IBUKA, Saitama (JP); Hiroyuki INUDUKA, Saitama (JP)

(21) Appl. No.: 18/010,084

(22) PCT Filed: Jun. 15, 2021
(86) PCT No.: PCT/JP2021/022598

§ 371 Date: May 1, 2023

(30) Foreign Application Priority Data
Jun. 15, 2020 (JP) 2020-103275
Mar. 9, 2021 (JP) 2021-037782

(51) Int. Cl. H10K 85/60; C07D 487/16; C09K 11/06

(52) U.S. Cl. CPC H10K 85/6572; C07D 487/16; C09K 11/06; H10K 85/654; H10K 50/11

(57) ABSTRACT

In order to provide an organic compound that can be suitably used as a luminescent material for a display, and an organic light emitting device containing such an organic compound, an organic compound in accordance with an embodiment of the present invention has a lone electron-pair and a π E electron orbit, and in the organic compound, an energy gap ΔE_{ST} obtained by subtracting an energy level E_{T1} of a lowest triplet excitation state T₁ from an energy level E_{S1} of a lowest singlet excitation mode S₁ is −0.20 eV≤ΔE_{ST}<0.0090 eV.

### TECHNICAL FIELD

The present invention relates to an organic compound that can be used as a luminescent material, and an organic light emitting device containing such an organic compound.

### BACKGROUND ART

An organic light emitting diode is an example of an organic light emitting device utilizing an organic electroluminescent (hereinafter referred to as “organic EL”) material constituted by an organic compound. Even though displays and lighting devices equipped with organic light emitting diodes are currently becoming available in the market, there is a high need for new organic EL materials with higher light emission efficiency. The organic EL material is an example of a luminescent material. Organic EL materials include fluorescent materials and phosphorescent materials. Theoretical internal quantum efficiency of a phosphorescent material is as high as four times that of a fluorescent material. Therefore, research and development of phosphorescent materials have preceded from the viewpoint of enhancing internal quantum efficiency.

### CITATION LIST

### Patent Literature

[Patent Literature 1]
International Publication No. WO 2015/159971

### Non-Patent Literature

[Non-patent Literature 1]
Hiroki Uoyama et al. “Highly efficient organic light-emitting diodes from delayed fluorescence”, Nature, 2012, 492, 234.
[Non-patent Literature 2]
Johannes Ehrmaier et. al., “Singlet-Triplet Inversion in Heptazine and in Polymeric Carbon Nitrides”, The Journal of Physical Chemistry A, 123, 8099-8108 (2019)

### SUMMARY OF INVENTION

### Technical Problem

However, the phosphorescent material contains an expensive metal such as iridium, and this poses a problem of high cost.

(Regarding Patent Literature 1 and Non-patent Literature 1)

As a luminescent material whose cost is lower than that of a phosphorescent material containing an expensive metal such as iridium, thermally activated delayed fluorescent materials disclosed in Patent Literature 1 and Non-patent Literature 1 are known. Hereinafter, the thermally activated delayed fluorescent material is referred to as “TADF material”.

The TADF material is configured such that an energy gap ΔE_{ST} obtained by subtracting an energy rank E_{T1} of a lowest triplet excitation state T₁ from an energy level E_{S1} of a lowest singlet excitation state S₁ is small (e.g., approximately 100 meV). The TADF material utilizes the lowest triplet excitation state T₁, in which the TADF material loses its activity as heat otherwise, as delayed fluorescence by thermally inducing reverse intersystem crossing from the lowest triplet excitation state T₁ to the lowest singlet excitation state S₁. As a result, it is possible, in principle, to increase internal quantum efficiency of the organic EL material to 100%.

By reducing ΔE_{ST} to be equivalent to energy at room temperature, reverse intersystem crossing is promoted, and a luminescence lifetime of delayed fluorescence is successfully shortened to several microseconds. This luminescence lifetime is equivalent to that of conventional phosphorescent materials.

However, when it is assumed that the TADF material is used in a display, the luminescence lifetime of the TADF material is, let it be said, far from the practical level. The luminescence lifetime of the TADF material is approximately three orders of magnitude longer than a typical luminescence lifetime of organic EL materials used in displays provided in the market.

This long luminescence lifetime causes deterioration of the TADF material due to an increase in triplet exciton density in the TADF material, and causes a decrease in light emission efficiency during light emission at high luminance.

(Regarding Non-Patent Literature 2)

Due to exchange interaction in the excited state, the energy rank E_{T1} of the lowest triplet excitation state T₁ becomes lower than the energy level E_{S1} of the lowest singlet excitation mode. In other words, ΔE_{ST} is positive.

Meanwhile, an organic compound in which ΔE_{ST} obtained by calculation is negative has been reported (e.g., see Non-patent Literature 2). The organic compound disclosed in Non-patent Literature 2 has ΔE_{ST}<−0.23 eV (see Table 3 of Non-patent Literature 2). Thus, ΔE_{ST} that is negative and has a large absolute value belongs to a region called “Marcus inverted region”. It is considered that a rate constant of reverse intersystem crossing from the lowest triplet excitation state T₁ to the lowest singlet excitation state S₁ is smaller in an organic EL material which belongs to the Marcus inverted region. The inventors of the present application have confirmed that such an organic EL material experimentally exhibits extremely low light emission intensity and luminescent quantum yield. Therefore, it is not realistic to utilize the organic EL material which belongs to the region called Marcus inverted region as a luminescent material for displays.

An aspect of the present invention is accomplished in view of the problem, and its object is to provide an organic compound which can be suitably used as a luminescent material for a display, and an organic light emitting device containing such an organic compound.

### Solution to Problem

In order to attain the object, an organic compound in accordance with a first aspect of the present invention has a lone electron-pair and a π electron orbit, in which an energy gap ΔE_{ST} obtained by subtracting an energy rank E_{T1} of a lowest triplet excitation state from an energy level E_{S1} of a lowest singlet excitation state is −0.20 eV ΔE_{ST}<0.0090 eV.

The organic compound in accordance with a second aspect of the present invention employs, in addition to the feature of the organic compound in accordance with the first aspect, a feature in which a radiative decay rate constant kᵣ is 1.0×10⁶s⁻¹<kᵣ.

The organic compound in accordance with a third aspect of the present invention employs, in addition to the feature of the organic compound in accordance with the first aspect or the second aspect, a feature in which an oscillator strength f is 0.0050<f.

The organic compound in accordance with a fourth aspect of the present invention employs, in addition to the feature of the organic compound in accordance with any one of the first aspect through third aspect, a feature in which the organic compound is a heptazine derivative that is represented by Formula (1) below and that has three arbitrary substituents R1, R2, and R3 which are independent of each other.

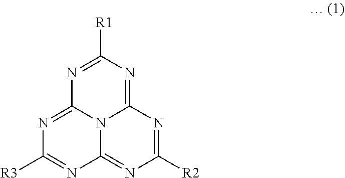

The organic compound in accordance with a fifth aspect of the present invention employs, in addition to the feature of the organic compound in accordance with the fourth aspect, a feature in which the substituents R1, R2, and R3 are constituted by two types of substituents.

The organic compound in accordance with a sixth aspect of the present invention employs, in addition to the feature of the organic compound in accordance with the fourth aspect, a feature in which the substituents R1, R2, and R3 are constituted by three different types of substituents.

The organic compound in accordance with a seventh aspect of the present invention employs, in addition to the feature of the organic compound in accordance with the fourth aspect, a feature in which the substituents R1, R2, and R3 are constituted by one type of substituent.

In order to attain the object, an organic compound in accordance with an eighth aspect of the present invention has a lone electron-pair and a n electron orbit, in which: the organic compound is a heptazine derivative that is represented by Formula (1) below and that has three arbitrary substituents R1, R2, and R3 which are independent of each other; and the substituents R1, R2, and R3 are constituted by two types or three types of substituents.

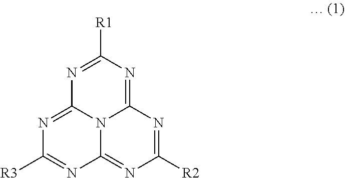

The organic light emitting device in accordance with a ninth aspect of the present invention contains the organic compound in accordance with any one of the first aspect through the eighth aspect of the present invention.

The organic light emitting device in accordance with a tenth aspect of the present invention employs, in addition to the feature of the organic light emitting device in accordance with the ninth aspect, a feature of including a luminescent layer that contains a host compound and the organic compound which functions as a dopant compound.

In order to attain the object, the organic light emitting device in accordance with an eleventh aspect of the present invention includes a luminescent layer that contains a dopant compound and a host compound. In the organic light emitting device, the host compound is an organic compound which has a lone electron-pair and a π electron orbit and in which an energy gap ΔE_{ST} obtained by subtracting an energy rank E_{T1} of a lowest triplet excitation state T₁ from an energy level E_{S1} of a lowest singlet excitation mode S₁ is negative or 0 eV ΔE_{ST}<0.0090 eV.

In order to attain the object, the organic light emitting device in accordance with a twelfth aspect of the present invention includes a luminescent layer that contains a dopant compound and a host compound. In the organic light emitting device, the host compound is a heptazine derivative that has a lone electron-pair and a π electron orbit, that is represented by Formula (1) below, and that includes an arbitrary substituent R1.

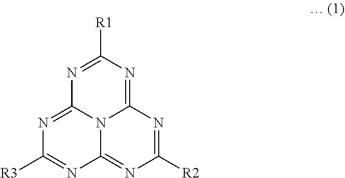

### Advantageous Effects of Invention

An aspect of the present invention can provide an organic compound which can be suitably used as a luminescent material for a display, and an organic light emitting device containing such an organic compound.

### BRIEF DESCRIPTION OF DRAWINGS

FIG. 1 a schematic view illustrating an energy level in an organic compound in accordance with an embodiment of the present invention.

FIG. 2 is a graph indicating an emission spectrum, temperature dependence of transient luminescent decay, and temperature dependence of a rate constant k_{DF} in a mixed thin film containing PPF and an organic compound A which is a first example of the present invention.

FIG. 3 is a graph indicating an emission spectrum, temperature dependence of transient luminescent decay, and temperature dependence of a rate constant k_{DF} in a toluene solution of an organic compound A which is a first referential example of the present invention.

FIG. 4 is a graph indicating a correlation between an energy gap ΔE_{ST} and an oscillator strength f in each of organic compounds 1 through 38 which are a second referential example group of the present invention.

FIG. 5 is a scatter diagram indicating a correlation between an energy gap ΔE_{ST} and an oscillator strength f in each of organic compounds pX-Y which are a third example group of the present invention.

FIG. 6 is a table indicating energy gaps ΔE_{ST} and oscillator strengths f of 1st through 200th organic compounds pX-Y in ascending order of energy gap ΔE_{ST} among the organic compounds pX-Y which are the third example group of the present invention.

FIG. 7 is a table indicating energy gaps ΔE_{ST} and oscillator strengths f of 201st through 400th organic compounds pX-Y in ascending order of energy gap ΔE_{ST} among the organic compounds pX-Y which are the third example group of the present invention.

FIG. 8 is a table indicating energy gaps ΔE_{ST} and oscillator strengths f of 401st through 600th organic example group of the present invention.

FIG. 9 is a table indicating energy gaps ΔE_{ST} and oscillator strengths f of 601st through 800th organic compounds pX-Y in ascending order of energy gap ΔE_{ST} among the organic compounds pX-Y which are the third example group of the present invention.

FIG. 10 is a table indicating energy gaps ΔE_{ST} and oscillator strengths f of 801st through 1000th organic compounds pX-Y in ascending order of energy gap ΔE_{ST} among the organic compounds pX-Y which are the third example group of the present invention.

FIG. 11 is a table indicating energy gaps ΔE_{ST} and oscillator strengths f of 1001st through 1200th organic compounds pX-Y in ascending order of energy gap ΔE_{ST} among the organic compounds pX-Y which are the third example group of the present invention.

FIG. 12 is a table indicating energy gaps ΔE_{ST} and oscillator strengths f of 1201st through 1400th organic compounds pX-Y in ascending order of energy gap ΔE_{ST} among the organic compounds pX-Y which are the third example group of the present invention.

FIG. 13 is a table indicating energy gaps ΔE_{ST} and oscillator strengths f of 1401st through 1600th organic example group of the present invention.

FIG. 14 is a table indicating energy gaps ΔE_{ST} and oscillator strengths f of 1601st through 1800th organic compounds pX-Y in ascending order of energy gap ΔE_{ST} among the organic compounds pX-Y which are the third example group of the present invention.

FIG. 15 is a table indicating energy gaps ΔE_{ST} and oscillator strengths f of 1801st through 2000th organic compounds pX-Y in ascending order of energy gap ΔE_{ST} among the organic compounds pX-Y which are the third example group of the present invention.

FIG. 16 is a table indicating energy gaps ΔE_{ST} and oscillator strengths f of 2001st through 2200th organic compounds pX-Y in ascending order of energy gap ΔE_{ST} among the organic compounds pX-Y which are the third example group of the present invention.

FIG. 17 is a table indicating energy gaps ΔE_{ST} and oscillator strengths f of 2201st through 2400th organic compounds pX-Y in ascending order of energy gap ΔE_{ST} among the organic compounds pX-Y which are the third example group of the present invention.

FIG. 18 is a table indicating energy gaps ΔE_{ST} and oscillator strengths f of 2401st through 2600th organic example group of the present invention.

FIG. 19 is a table indicating energy gaps ΔE_{ST} and oscillator strengths f of 2601st through 2800th organic compounds pX-Y in ascending order of energy gap ΔE_{ST} among the organic compounds pX-Y which are the third example group of the present invention.

FIG. 20 is a table indicating energy gaps ΔE_{ST} and oscillator strengths f of 2801st through 3000th organic compounds pX-Y in ascending order of energy gap ΔE_{ST} among the organic compounds pX-Y which are the third example group of the present invention.

FIG. 21 is a table indicating energy gaps ΔE_{ST} and oscillator strengths f of 3001st through 3200th organic compounds pX-Y in ascending order of energy gap ΔE_{ST} among the organic compounds pX-Y which are the third example group of the present invention.

FIG. 22 is a table indicating energy gaps ΔE_{ST} and oscillator strengths f of 3201st through 3400th organic compounds pX-Y in ascending order of energy gap ΔE_{ST} among the organic compounds pX-Y which are the third example group of the present invention.

FIG. 23 is a table indicating energy gaps ΔE_{ST} and oscillator strengths f of 3401st through 3600th organic example group of the present invention.

FIG. 24 is a table indicating energy gaps ΔE_{ST} and oscillator strengths f of 3601st through 3800th organic compounds pX-Y in ascending order of energy gap ΔE_{ST} among the organic compounds pX-Y which are the third example group of the present invention.

FIG. 25 is a table indicating energy gaps ΔE_{ST} and oscillator strengths f of 3801st through 4000th organic compounds pX-Y in ascending order of energy gap ΔE_{ST} among the organic compounds pX-Y which are the third example group of the present invention.

FIG. 26 is a table indicating energy gaps ΔE_{ST} and oscillator strengths f of 4001st through 4200th organic compounds pX-Y in ascending order of energy gap ΔE_{ST} among the organic compounds pX-Y which are the third example group of the present invention.

FIG. 27 is a table indicating energy gaps ΔE_{ST} and oscillator strengths f of 4201st through 4400th organic compounds pX-Y in ascending order of energy gap ΔE_{ST} among the organic compounds pX-Y which are the third example group of the present invention.

FIG. 28 is a table indicating energy gaps ΔE_{ST} and oscillator strengths f of 4401st through 4600th organic example group of the present invention.

FIG. 29 is a table indicating energy gaps ΔE_{ST} and oscillator strengths f of 4601st through 4800th organic compounds pX-Y in ascending order of energy gap ΔE_{ST} among the organic compounds pX-Y which are the third example group of the present invention.

FIG. 30 is a table indicating energy gaps ΔE_{ST} and oscillator strengths f of 4801st through 5000th organic compounds pX-Y in ascending order of energy gap ΔE_{ST} among the organic compounds pX-Y which are the third example group of the present invention.

FIG. 31 is a table indicating energy gaps ΔE_{ST} and oscillator strengths f of 5001st through 5200th organic compounds pX-Y in ascending order of energy gap ΔE_{ST} among the organic compounds pX-Y which are the third example group of the present invention.

FIG. 32 is a table indicating energy gaps ΔE_{ST} and oscillator strengths f of 5201st through 5400th organic compounds pX-Y in ascending order of energy gap ΔE_{ST} among the organic compounds pX-Y which are the third example group of the present invention.

FIG. 33 is a table indicating energy gaps ΔE_{ST} and oscillator strengths f of 5401st through 5600th organic example group of the present invention.

FIG. 34 is a table indicating energy gaps ΔE_{ST} and oscillator strengths f of 5601st through 5800th organic compounds pX-Y in ascending order of energy gap ΔE_{ST} among the organic compounds pX-Y which are the third example group of the present invention.

FIG. 35 is a table indicating energy gaps ΔE_{ST} and oscillator strengths f of 5801st through 6000th organic compounds pX-Y in ascending order of energy gap ΔE_{ST} among the organic compounds pX-Y which are the third example group of the present invention.

FIG. 36 is a table indicating energy gaps ΔE_{ST} and oscillator strengths f of 6001st through 6200th organic compounds pX-Y in ascending order of energy gap ΔE_{ST} among the organic compounds pX-Y which are the third example group of the present invention.

FIG. 37 is a table indicating energy gaps ΔE_{ST} and oscillator strengths f of 6201st through 6400th organic compounds pX-Y in ascending order of energy gap ΔE_{ST} among the organic compounds pX-Y which are the third example group of the present invention.

FIG. 38 is a table indicating energy gaps ΔE_{ST} and oscillator strengths f of 6401st through 6600th organic example group of the present invention.

FIG. 39 is a table indicating energy gaps ΔE_{ST} and oscillator strengths f of 6601st through 6800th organic compounds pX-Y in ascending order of energy gap ΔE_{ST} among the organic compounds pX-Y which are the third example group of the present invention.

FIG. 40 is a table indicating energy gaps ΔE_{ST} and oscillator strengths f of 6801st through 7000th organic compounds pX-Y in ascending order of energy gap ΔE_{ST} among the organic compounds pX-Y which are the third example group of the present invention.

FIG. 41 is a table indicating energy gaps ΔE_{ST} and oscillator strengths f of 7001st through 7200th organic compounds pX-Y in ascending order of energy gap ΔE_{ST} among the organic compounds pX-Y which are the third example group of the present invention.

FIG. 42 is a table indicating energy gaps ΔE_{ST} and oscillator strengths f of 7201st through 7400th organic compounds pX-Y in ascending order of energy gap ΔE_{ST} among the organic compounds pX-Y which are the third example group of the present invention.

FIG. 43 is a table indicating energy gaps ΔE_{ST} and oscillator strengths f of 7401st through 7600th organic example group of the present invention.

FIG. 44 is a table indicating energy gaps ΔE_{ST} and oscillator strengths f of 7601st through 7800th organic compounds pX-Y in ascending order of energy gap ΔE_{ST} among the organic compounds pX-Y which are the third example group of the present invention.

FIG. 45 is a table indicating energy gaps ΔE_{ST} and oscillator strengths f of 7801st through 8000th organic compounds pX-Y in ascending order of energy gap ΔE_{ST} among the organic compounds pX-Y which are the third example group of the present invention.

FIG. 46 is a table indicating energy gaps ΔE_{ST} and oscillator strengths f of 8001st through 8200th organic compounds pX-Y in ascending order of energy gap ΔE_{ST} among the organic compounds pX-Y which are the third example group of the present invention.

FIG. 47 is a table indicating energy gaps ΔE_{ST} and oscillator strengths f of 8201st through 8400th organic compounds pX-Y in ascending order of energy gap ΔE_{ST} among the organic compounds pX-Y which are the third example group of the present invention.

FIG. 48 is a table indicating energy gaps ΔE_{ST} and oscillator strengths f of 8401st through 8600th organic example group of the present invention.

FIG. 49 is a table indicating energy gaps ΔE_{ST} and oscillator strengths f of 8601st through 8800th organic compounds pX-Y in ascending order of energy gap ΔE_{ST} among the organic compounds pX-Y which are the third example group of the present invention.

FIG. 50 is a table indicating energy gaps ΔE_{ST} and oscillator strengths f of 8801st through 9000th organic compounds pX-Y in ascending order of energy gap ΔE_{ST} among the organic compounds pX-Y which are the third example group of the present invention.

FIG. 51 is a table indicating energy gaps ΔE_{ST} and oscillator strengths f of 9001st through 9200th organic compounds pX-Y in ascending order of energy gap ΔE_{ST} among the organic compounds pX-Y which are the third example group of the present invention.

FIG. 52 is a table indicating energy gaps ΔE_{ST} and oscillator strengths f of 9201st through 9400th organic compounds pX-Y in ascending order of energy gap ΔE_{ST} among the organic compounds pX-Y which are the third example group of the present invention.

FIG. 53 is a table indicating energy gaps ΔE_{ST} and oscillator strengths f of 9401st through 9600th organic example group of the present invention.

FIG. 54 is a table indicating energy gaps ΔE_{ST} and oscillator strengths f of 9601st through 9800th organic compounds pX-Y in ascending order of energy gap ΔE_{ST} among the organic compounds pX-Y which are the third example group of the present invention.

FIG. 55 is a table indicating energy gaps ΔE_{ST} and oscillator strengths f of 9801st through 10000th organic compounds pX-Y in ascending order of energy gap ΔE_{ST} among the organic compounds pX-Y which are the third example group of the present invention.

FIG. 56 is a table indicating energy gaps ΔE_{ST} and oscillator strengths f of 10001st through 10006th organic compounds pX-Y in ascending order of energy gap ΔE_{ST} among the organic compounds pX-Y which are the third example group of the present invention.

FIG. 57 is a graph indicating an emission spectrum in a toluene solution of an organic compound C which is an example of the present invention.

FIG. 58 is a graph indicating temperature dependence of transient luminescent decay in a toluene solution of the organic compound C which is an example of the present invention.

FIG. 59 is a graph indicating temperature dependence of a rate constant k_{DF} of delayed fluorescence in a toluene solution of the organic compound C which is an example of the present invention.

FIG. 60 is a graph indicating an emission spectrum in a toluene solution of an organic compound D which is an example of the present invention.

FIG. 61 is a graph indicating temperature dependence of transient luminescent decay in a toluene solution of the organic compound D which is an example of the present invention.

FIG. 62 is a graph indicating temperature dependence of a rate constant k_{DF} of delayed fluorescence in a toluene solution of the organic compound D which is an example of the present invention.

FIG. 63 is a graph indicating an emission spectrum in a toluene solution of an organic compound E which is an example of the present invention.

FIG. 64 is a graph indicating transient luminescent decay in a toluene solution of the organic compound E which is an example of the present invention.

FIG. 65 is a graph indicating an emission spectrum of an organic light emitting device using the organic compound C which is an example of the present invention.

FIG. 66 is a graph indicating current density-voltage-luminance characteristics of the organic light emitting device using the organic compound C which is an example of the present invention.

FIG. 67 is a graph indicating external quantum efficiency-luminance characteristics of the organic light emitting device using the organic compound C which is an example of the present invention.

FIG. 68 is a graph indicating transient luminescent decay of the organic light emitting device using the organic compound C which is an example of the present invention and an organic light emitting device using 4CzIPN.

### DESCRIPTION OF EMBODIMENTS

[Organic Compound]

<Overview>

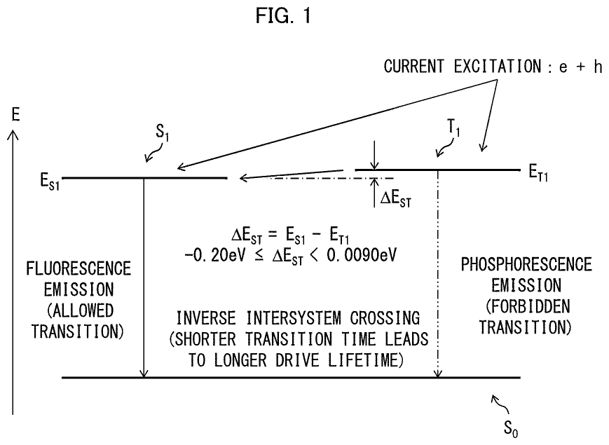

An organic compound in accordance with an aspect of the present invention is an organic compound having a lone electron-pair and a n electron orbit. Hereinafter, the organic compound in accordance with an aspect of the present invention is referred to as “present organic compound”. The present organic compound can take, at least, a ground state S₀, a lowest singlet excitation mode S₁, and a lowest triplet excitation state T₁ (see FIG. 1). In a case where electrons and positive holes are induced in the present organic compound, some of the electrons and the positive holes are excited to be a lowest singlet excitation mode S₁, and the remaining majority of the electrons and the positive holes are excited to be a lowest triplet excitation state T₁. Hereinafter, the induced electrons and positive holes are collectively referred to as “carriers”.

The present organic compound is configured such that an energy gap ΔE_{ST} obtained by subtracting an energy rank E_{T1} of a lowest triplet excitation state T₁ from an energy level E_{S1} of a lowest singlet excitation mode S₁ is −0.20 eV ΔE_{ST}<0.0090 eV. FIG. 1 illustrates a state in which the energy rank E_{T1} is greater than the energy level E_{S1}, that is, a state in which the energy gap ΔE_{ST} is positive.

In the present organic compound, the energy gap ΔE_{ST} is preferably negative, that is, the present organic compound is preferably configured such that −0.20 eV ΔE_{ST}<0 eV holds true.

In the present organic compound, the radiative decay rate constant kᵣ is preferably 1.0×10⁶s⁻¹<kᵣ.

In the present organic compound, the oscillator strength f is preferably 0.0050<f.

The above described energy gap ΔE_{ST}, radiative decay rate constant kᵣ, and oscillator strength f are each indicated in two-digit significant figures. In a case where the significant figures of the energy gap ΔE_{ST}, the radiative decay rate constant kᵣ, and the oscillator strength f are each three or more digits, the significant figures are each made a two-digit figure by rounding off the third digit of the significant figure.

<Advantage of Organic Compound>

The lowest triplet excitation state T₁ is an unstable excitation state. Therefore, for example, in a case where the present organic compound is used as a luminescent material for a display including an organic light emitting diode, deterioration of the organic compound is more likely to progress as a period of time for which excited carriers remain in the lowest triplet excitation state T₁ increases. Accordingly, a drive lifetime, during which the present organic compound can be driven as a luminescent material, tends to become shorter.

In the present organic compound, the energy gap ΔE_{ST} is less than 0.0090 eV. Therefore, as compared with the TADF materials disclosed in Patent Literature 1 and Non-patent Literature 1, reverse intersystem crossing from the lowest triplet excitation state T₁ to the lowest singlet excitation mode S₁ occurs more easily. That is, a rate constant k_{RISC} in reverse intersystem crossing in the present organic compound is greater than a rate constant k_{RISC} of the TADF materials disclosed in Patent Literature 1 and Non-patent Literature 1. That is, the present organic compound makes it possible to shorten the period of time for which excited carriers remain in the lowest triplet excitation state T₁, as compared with the TADF materials disclosed in Patent Literature 1 and Non-patent Literature 1.

The luminescence lifetime of fluorescence emission caused by recombination of carriers in the lowest singlet excitation mode S₁ is shorter than the luminescence lifetime of fluorescence emission caused by recombination of carriers from the lowest triplet excitation state T₁. Therefore, the present organic compound makes it possible to shorten the luminescence lifetime, as compared with the TADF materials disclosed in Patent Literature 1 and Non-patent Literature 1.

The present organic compound configured as described above can enhance durability as compared with the TADF materials disclosed in Patent Literature 1 and Non-patent Literature 1, and can thereby increase the drive lifetimes of an organic light emitting diode and a display each using the present organic compound.

In the present organic compound, the energy gap ΔE_{ST} is not less than −0.20 eV. Therefore, as compared with the organic compound disclosed in Non-patent Literature 2, the rate constant k_{RISC} can be increased, and the light emission intensity and luminescent quantum yield can be increased.

An organic compound in which the energy gap ΔE_{ST} is clearly lower than −0.20 eV has a negative energy gap ΔE_{ST}, whose absolute value is an excessively large. Therefore, such an organic compound belongs to the Marcus inverted region. Organic compounds belonging to the Marcus inverted region are expected, from a calculation result, to have a low rate constant k_{RISC}. It has been confirmed experimentally that organic compounds belonging to the Marcus inverted region have extremely low light emission intensity and luminescent quantum yield. Therefore, it is not realistic to utilize an organic compound belonging to the Marcus inverted region as a luminescent material for displays.

Therefore, the present organic compound can be suitably used as a luminescent material for a display including an organic light emitting diode, as compared with the TADF materials disclosed in Patent Literature 1 and Non-patent Literature 1 and the organic compound disclosed in Non-patent Literature 2. The organic light emitting diode is an aspect of the organic light emitting device, and an organic light emitting diode containing the present organic compound is encompassed in the scope of the present invention.

<Upper Limit and Lower Limit of Energy Gap ΔE_{ST}>

(Preferable Lower Limit of Energy Gap ΔE_{ST})

Assuming that reverse intersystem crossing in an organic compound is non-adiabatic transition based on weak spin-orbit interaction (H_{SO}) of the organic compound, a rate constant k_{RISC} thereof can be expressed by a mathematical formula (1) which is a formula of a Marcus theory type (see Aizawa, N., Harabuchi, Y., Maeda, S., & Pu, Y.-J. Kinetic Prediction of Reverse Intersystem Crossing in Organic Donor-Acceptor Molecules. ChemRxiv. Preprint. https://doi.org/10.26434/chemrxiv.12203240.v1).

$${k}_{RISC} = \frac{2 π}{ℏ} {\vert {H}_{SO} \vert}^{2} {( 4 π λ {k}_{B} T )}^{- \frac{1}{2}} exp ( \frac{- {E}_{A}}{{k}_{B} T} ) \qquad ( 1 )$$

In the mathematical formula (1),
ℏ

is a Dirac constant, k_{B} is a Boltzmann constant, T is an absolute temperature, λ is rearrangement energy, and E_{A} is activation energy. Assuming a harmonic oscillator in the lowest singlet excitation mode S₁ and the lowest triplet excitation state T₁, the activation energy E_{A} can be expressed by a mathematical formula (2) in terms of the rearrangement energy λ and the energy gap ΔE_{ST}.

$${E}_{A} = \frac{{( Δ {E}_{ST} + λ )}^{2}}{4 λ} \qquad ( 2 )$$

From the mathematical formulae (1) and (2), the rate constant k_{RISC} is maximized when ΔE_{ST}+λ=0. A theoretical value of λ obtained by TDDFT calculation is not less than 0.050 eV and not greater than 0.20 eV in a typical TADF material (see the above described Aizawa et. al.), and is not less than 0.0030 eV and not greater than 0.10 eV in a heptazine derivative which is an example of the present organic compound. The present organic compound in which the lower limit of the energy gap ΔE_{ST} is −0.20 eV makes it possible to increase the rate constant k_{RISC}

In the organic compound in accordance with an aspect of the present invention, the energy gap ΔE_{ST} may be less than −0.20 eV.

(Upper limit of energy gap ΔE_{ST}) The rearrangement energy λ is always positive because the rearrangement energy λ is on the basis of most stable energy of the lowest triplet excitation state T₁. So far, the lowest ΔE_{ST} in an isolated single organic molecule is reported to be 0.009 eV (see Hironori Kaji et al. “Purely organic electroluminescent material realizing 100% conversion from electricity to light”, Nat. Commun. 6, 8476 (2015)). Exchange interaction between molecules is one of sources of the energy gap ΔE_{ST} Note that exchange interaction between molecules is smaller than exchange interaction within a molecule. In the present organic compound, the upper limit of the energy gap ΔE_{ST} is 0.0090 eV. Therefore, the present organic compound makes it possible to cause the rate constant k_{RISC} to be greater than those of the TADF materials disclosed in Patent Literature 1 and Non-patent Literature 1.

<Lower limit of radiative decay rate constant kᵣ>

In the present organic compound, the radiative decay rate constant kᵣ is preferably 1.0×10⁶s⁻¹<kᵣ≤1×10⁹s⁻¹. According to this configuration, it is possible to realize a quantum yield and a luminescence lifetime that are close to or equivalent to those of typical luminescent materials used in displays equipped with organic light emitting diodes which are provided to the market.

In the present organic compound, the oscillator strength f is preferably 0.0050<f. According to this configuration, it is possible to increase the intensity of fluorescence. Therefore, in a case where the present organic compound is used as a luminescent material constituting a luminescent layer of an organic light emitting diode, the luminance of an organic EL element can be increased.

<Wavelength of Fluorescence>

The wavelength λ (nm) of fluorescence emitted by the present organic compound is determined in accordance with an energy gap ΔE_{S01} (eV) obtained by subtracting an energy level E_{S0} of a ground state S₀ from an energy level E_{S1} of a lowest singlet excitation mode S₁. The wavelength λ is obtained by λ=1240/ΔE_{S01}.

In the present organic compound, the wavelength λ is not particularly limited.

### Preferable Example of Organic Compound

The following description will more specifically discuss a preferable example of the present organic compound. Note, however, that a chemical structure of the present organic compound is not limited to the following example, provided that the energy gap ΔE_{ST} is negative or satisfies a relation of 0 eV≤ΔE_{ST}<0.0090 eV. The present organic compound preferably satisfies a relation of −0.20 eV≤ΔE_{ST}<0.0090 eV.

In a preferable example, the present organic compound has a structure represented by Formula (2) below.

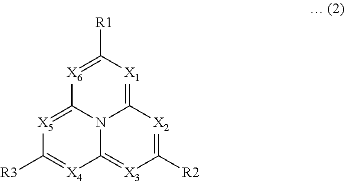

In Formula (2), R1, R2, and R3 (hereinafter, sometimes referred to as “R1 through R3”) are arbitrary substituents which are independent of each other. X1, X2, X3, X4, X5, and X6 (hereinafter, sometimes referred to as “X1 through X6”) are nitrogen atoms or CH which are independent of each other. In a case where X1 through X6 are nitrogen atoms, a preferable example of the present organic compound is a heptazine derivative.

In Formula (2) above, it is preferable that at least one of X1 through X6 is a nitrogen atom, it is more preferable that two or more or three or more of X1 through X6 are nitrogen atoms, and it is further preferable that all of X1 through X6 are nitrogen atoms. In a case where all of X1 through X6 are nitrogen atoms, the structure is represented by Formula (1) below.

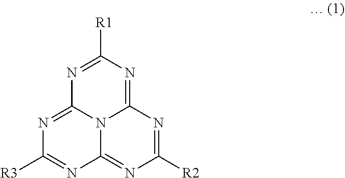

In Formula (1), the definitions of R1 through R3 are the same as those in Formula (2).

The following description will more specifically discuss preferable examples of R1 through R3 in Formulae (1) and (2).

R1 through R3 may be different substituents. In some cases, a structure is preferable in which two of R1 through R3 (e.g., R2 and R3, or R1 and R3) are identical substituents and the other one is a different substituent. That is, in some cases, R1 through R3 are preferably constituted by three different types of substituents, in other cases, R1 through R3 are preferably constituted by two types of substituents, or in other cases, R1 through R3 are preferably constituted by one type of substituent.

In particular, in some cases, by setting symmetry in a preferable example to be lower than D₃ₕ, an organic compound can be realized in which the energy gap ΔE_{ST} is negative, that is, which satisfies a relation of −0.20 eV ΔE_{ST}<0 eV and has a higher luminescent quantum yield.

### Examples of R1 Through R3

Examples of R1 through R3 are as follows.

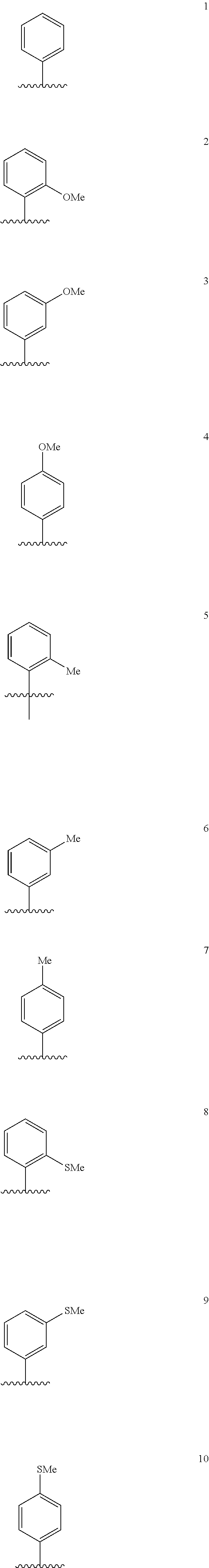

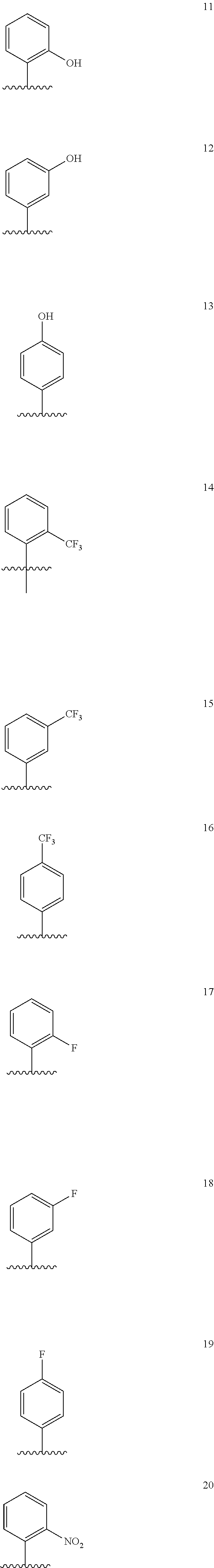

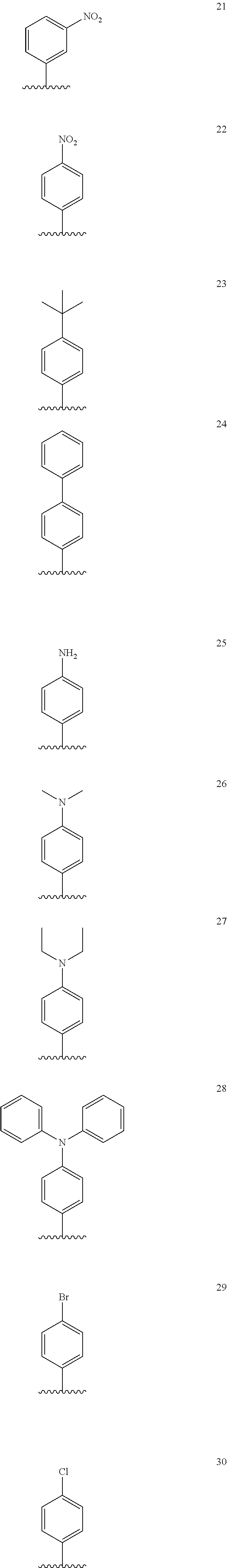

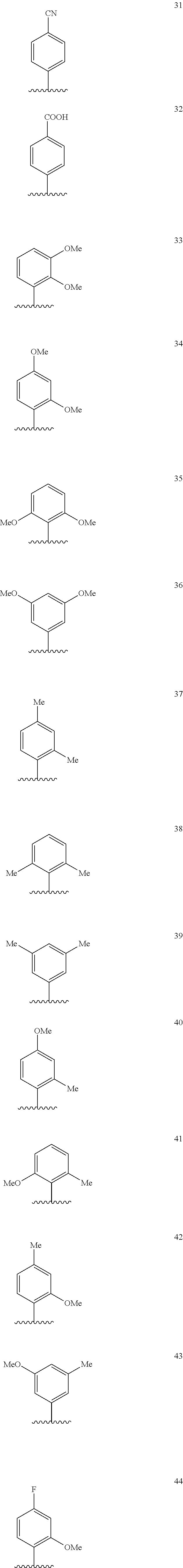

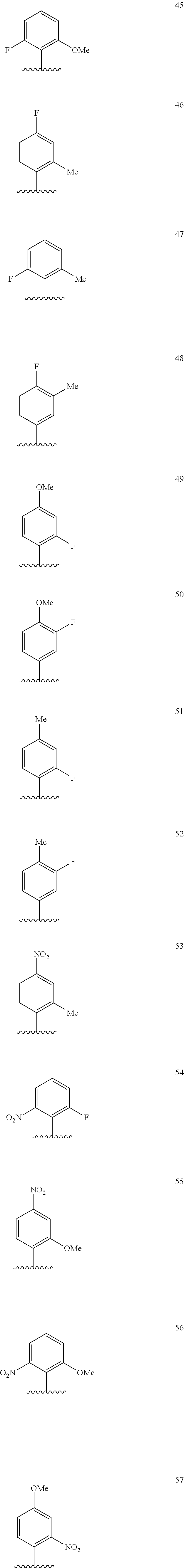

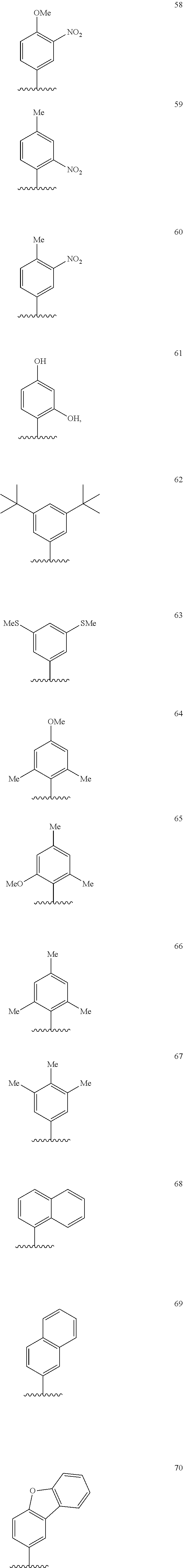

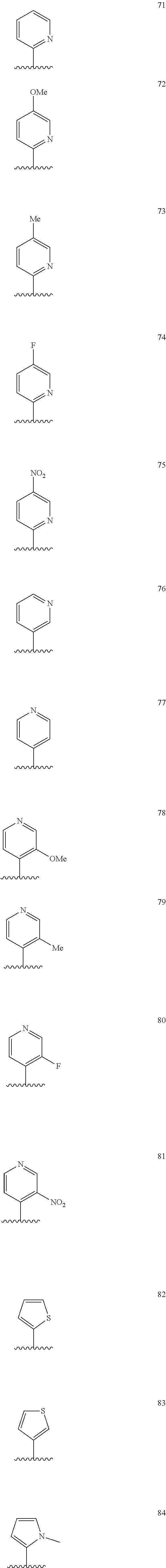

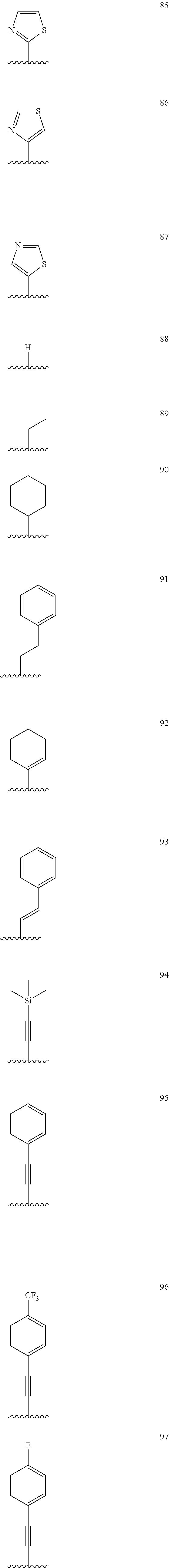

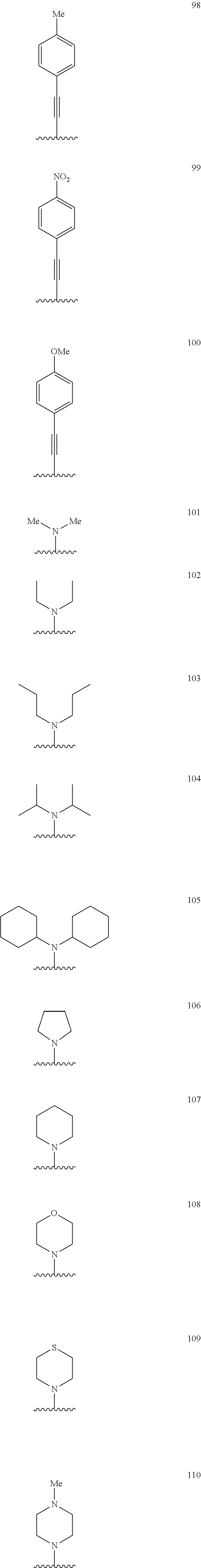

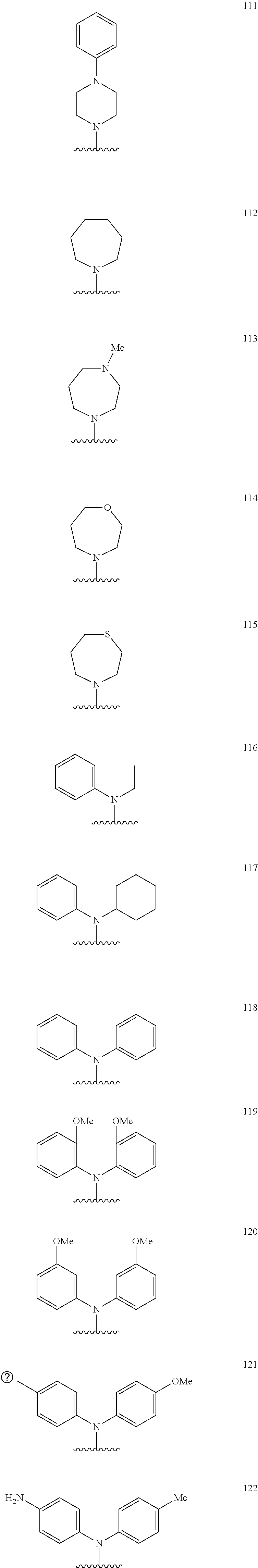

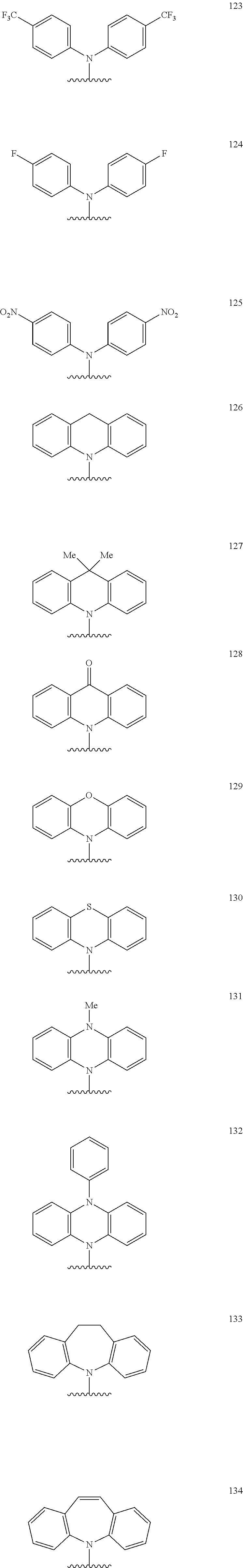

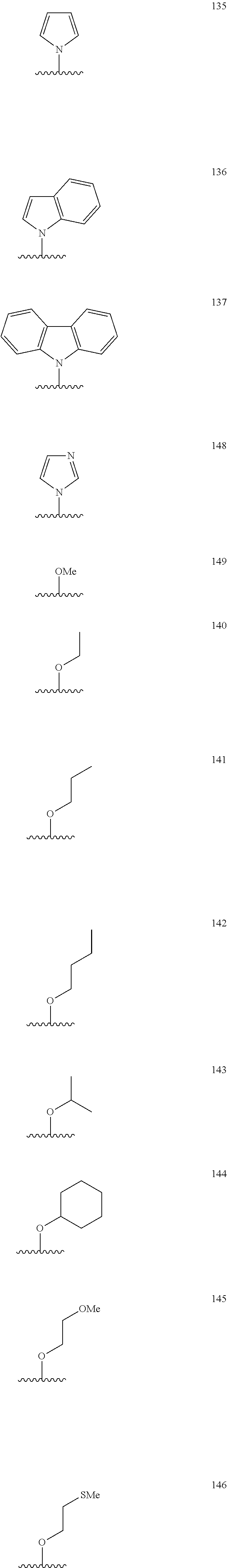

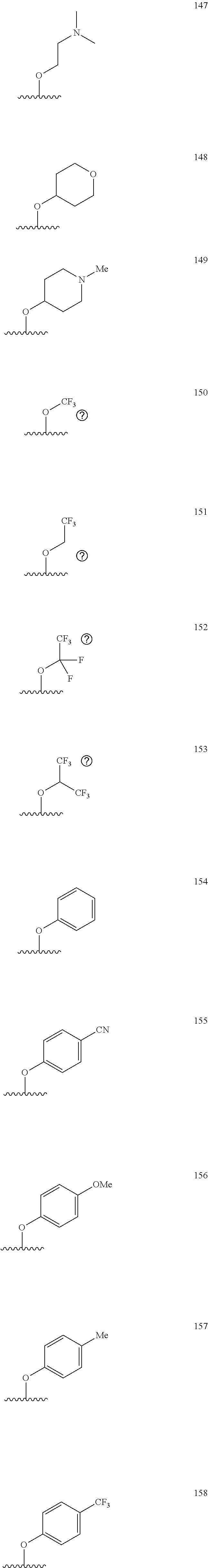

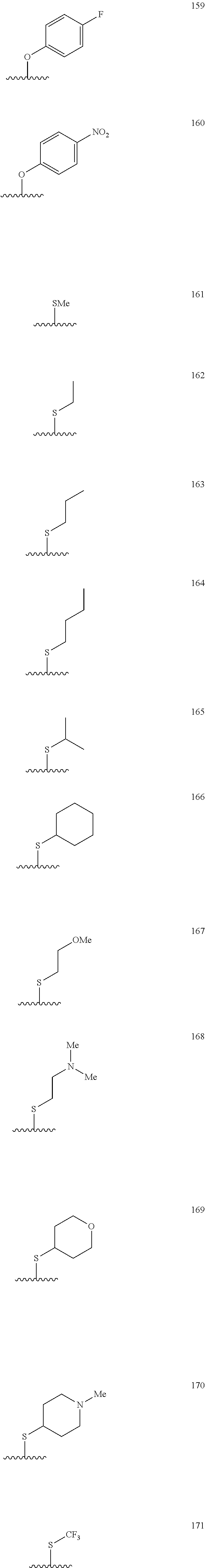

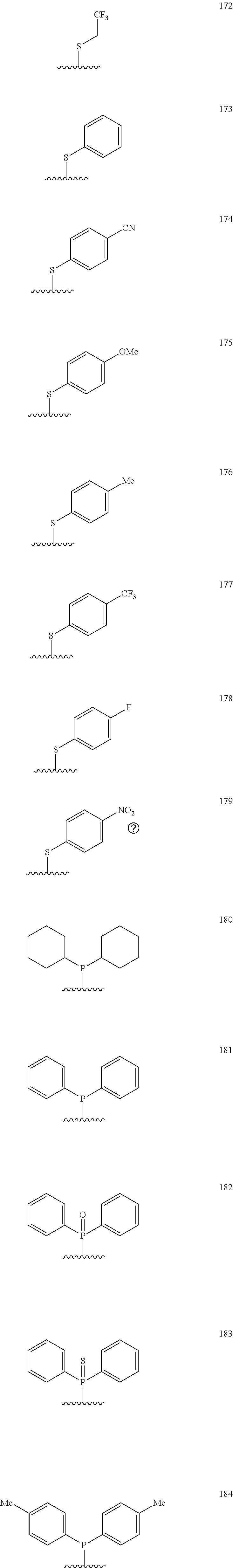

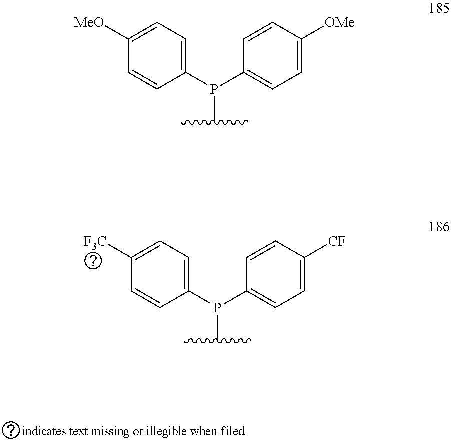

### Example of R1

In a more preferable example, R1 takes a structure represented by Formula (3).

—S—R31,—O—R31, or, —N—(R32)R33 (3)

In Formula (3), R31 through R33 are chain or cyclic hydrocarbon groups which are independent of each other and have a carbon number of 20 or less, and may each be substituted by a substituent. The chain or cyclic hydrocarbon groups may preferably have a carbon number of, for example, 10 or less. Further, R32 and R33 which are bonded to the same nitrogen (N) may be bonded to each other to form a ring structure.

As R31 through R33, specific examples of the chain or cyclic hydrocarbon groups include a chain alkyl group, a chain alkenyl group, a chain alkynyl group, a hydrocarbon ring group, and the like.

Examples of the chain alkyl group include those in the form of linear chain or branched chain having a carbon number of 20 or less, preferably 15 or less, more preferably 10 or less, and further preferably 5 or less, such as a methyl group, an ethyl group, a propyl group, an isopropyl group, a butyl group, an isobutyl group, a sec-butyl group, a tert-butyl group, a hexyl group, and an octyl group.

Examples of the chain alkenyl group include those in the form of linear chain or branched chain having a carbon number of 20 or less, preferably 15 or less, and more preferably 10 or less, such as a vinyl group, a propenyl group, a butenyl group, a 2-methyl-1-propenyl group, a hexenyl group, and an octenyl group.

Examples of the chain alkynyl group include those in the form of linear chain or branched chain having a carbon number of 20 or less, preferably 15 or less, and more preferably 10 or less, such as an ethynyl group, a propynyl group, a butynyl group, a 2-methyl-1-propynyl group, a hexynyl group, and an octynyl group.

Examples of the hydrocarbon ring group include cycloalkyl groups having a carbon number of 3 or more, preferably 5 or more, and a carbon number of 20 or less, preferably 15 or less, and more preferably 10 or less, such as a cyclopropyl group, a cyclohexyl group, and a tetradecahydroanthranil group; cycloalkenyl groups having a carbon number of 3 or more, preferably 5 or more, and a carbon number of 20 or less, preferably 15 or less, and more preferably 10 or less, such as a cyclohexenyl group; and aryl groups having a carbon number of 6 or more, and a carbon number of 18 or less, and preferably 10 or less, such as a phenyl group, an anthranil group, a phenanthryl group, and a ferrocenyl group.

The chain alkyl groups, chain alkenyl groups, chain alkynyl groups, hydrocarbon ring groups, or the like which are exemplified as R31 through R33, may have substituents. Substituents for the chain alkyl groups, chain alkenyl groups, or chain alkynyl groups include, for example, halogen groups (halogen atoms) such as a fluorine atom, a chlorine atom, a bromine atom, and an iodine atom.

Substituents for the hydrocarbon ring groups include, for example, an amino group (—NH₂), a nitro group (—NO₂), a cyano group (—CN), a hydroxyl group (—OH), an alkyl group, an alkyl halide group, an alkoxy group, and the like, in addition to the above described halogen groups. Examples of the alkoxy group include a methoxy group, an ethoxy group, a propoxy group, an isopropoxy group, a butoxy group, an isobutoxy group, a sec-butoxy group, a tert-butoxy group, a pentyloxy group, a hexyloxy group, an octyloxy group, and the like. Alkyl moieties such as an alkyl group, an alkyl halide group, and an alkoxy group have a carbon number of preferably 5 or less.

More specific examples of the structure represented by Formula (3) described above are as follows.

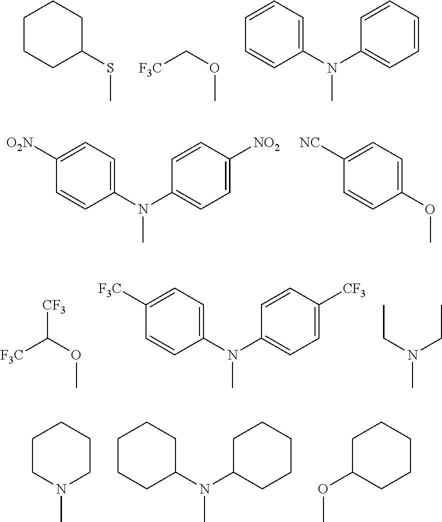

### Example of R2

In a more preferable example, R2 is selected from hydrocarbon ring groups or heterocyclic groups.

Examples of the hydrocarbon ring groups include cycloalkyl groups having a carbon number of 3 or more, preferably 5 or more, and a carbon number of 20 or less, preferably 15 or less, and more preferably 10 or less, such as a cyclopropyl group, a cyclohexyl group, and a tetradecahydroanthranil group; cycloalkenyl groups having a carbon number of 3 or more, preferably 5 or more, and a carbon number of 20 or less, preferably 15 or less, and more preferably 10 or less, such as a cyclohexenyl group; and aryl groups having a carbon number of 6 or more, and a carbon number of 18 or less, and preferably 10 or less, such as a phenyl group, an anthranil group, a phenanthryl group, and a ferrocenyl group.

Examples of the heterocyclic groups include heteroaryl groups each constituted by a 5 to 6-membered monocycle or a condensed ring in which two to six 5 to 6-membered rings are condensed; and heterocycloalkyl groups each constituted by a 5 to 6-membered monocycle or a condensed ring in which two to six 5 to 6-membered rings are condensed. Examples of heteroatoms include a nitrogen atom, an oxygen atom, a sulfur atom, and the like. Specific examples include: 5-membered monocycles such as a thienyl group; 6-membered monocycles such as a pyridyl group, a 1-piperidinyl group, a 2-piperidinyl group, and a 2-piperazinyl group; and condensed rings formed by condensation of two to six 5 to 6-membered rings such as a benzothienyl group, a carbazolyl group, a quinolinyl group, and an octahydroquinolinyl group.

Preferable examples of R2 are a phenyl group which may have one to five substituents, or a pyridyl group which may have one to four substituents (described later). In a case of having a substituent, the number of substituents is not particularly limited, and in some cases, the number of substituents is preferably one to three.

These hydrocarbon ring groups, heterocyclic groups, and the like may each have a substituent as described above. Examples of the substituents include: halogen groups (halogen atoms) such as a fluorine atom, a chlorine atom, a bromine atom, and an iodine atom; amino groups (—NH₂); nitro groups (—NO₂); cyano groups (—CN); hydroxyl groups (—OH); alkyl groups in the form of linear chain or branched chain having a carbon number of 20 or less, preferably 15 or less, more preferably 10 or less, and further preferably 5 or less, such as a methyl group, an ethyl group, a propyl group, an isopropyl group, a butyl group, an isobutyl group, a sec-butyl group, a tert-butyl group, a hexyl group, and an octyl group; alkyl halide groups; alkoxy groups; and the like. Examples of the alkoxy group include a methoxy group, an ethoxy group, a propoxy group, an isopropoxy group, a butoxy group, an isobutoxy group, a sec-butoxy group, a tert-butoxy group, a pentyloxy group, a hexyloxy group, an octyloxy group, and the like. Alkyl moieties such as an alkyl group, an alkyl halide group, and an alkoxy group have a carbon number of preferably 5 or less.

The following description will more specifically discuss preferable examples of R2, i.e., a phenyl group which may have one to five substituents or a pyridyl group which may have one to four substituents. The substituent possessed by the phenyl group or the pyridyl group is preferably the above described halogen group, hydroxyl group, alkyl group, alkyl halide group, or alkoxy group. In the phenyl group which may have one to five substituents, a preferable number of substituents is 0, 1, 2, or 3. In a case where the number of substituents is one, it is preferable that the substituent is at position 2 or position 4 of the phenyl group. In a case where the number of substituents is two, it is preferable that the substituents are at positions 2 and 4 or positions 2 and 6 of the phenyl group. In a case where the number of substituents is three, it is preferable that the substituents are at positions 2, 4, and 6 of the phenyl group. In a case where the number of substituents is two or three, two or three substituents selected from the group consisting of alkyl groups, alkoxy groups, and halogen groups may be preferable. In the pyridyl group which may have one to four substituents, a preferable number of substituents is 0, 1, 2, or 3, and 0, 1, or 2 may be more preferable.

More specific examples of R2 are as follows.

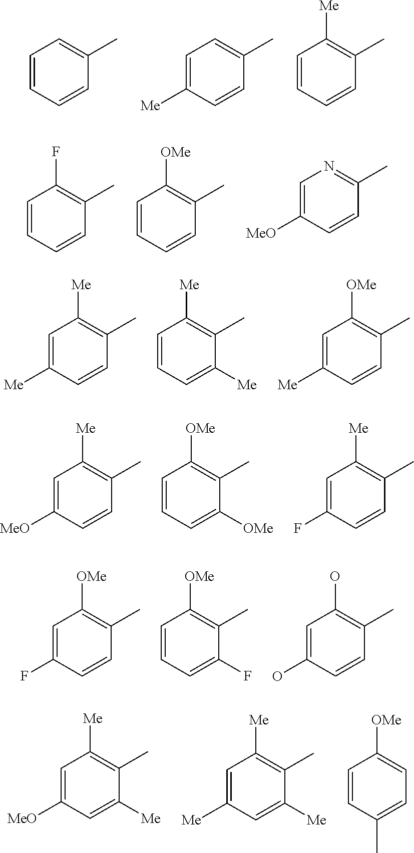

### Example of R3

In a more preferable example, R3 is selected from among the substituents exemplified as R1 and the substituents exemplified as R2. Although R1 through R3 may be different substituents, it is more preferable that R3 and R1 are identical substituents or that R3 and R2 are identical substituents.

### Examples of Preferable Combination of R1, R2, and R3

In an example of a preferable combination of R1, R2, and R3, R1 is a substituent satisfying Formula (3) above, and R2 and R3 are phenyl groups which may be substituted by one to three substituents. Here, R2 and R3 are more preferably identical groups. Further preferably, R1 is any of the following:

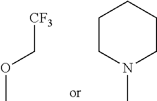

and R2 and R3 are a combination selected from among unsubstituted phenyl groups and phenyl groups which are substituted by one to three methyl groups (preferably, R2 and R3 are identical groups).

Particularly preferable examples of the present organic compound include the following.

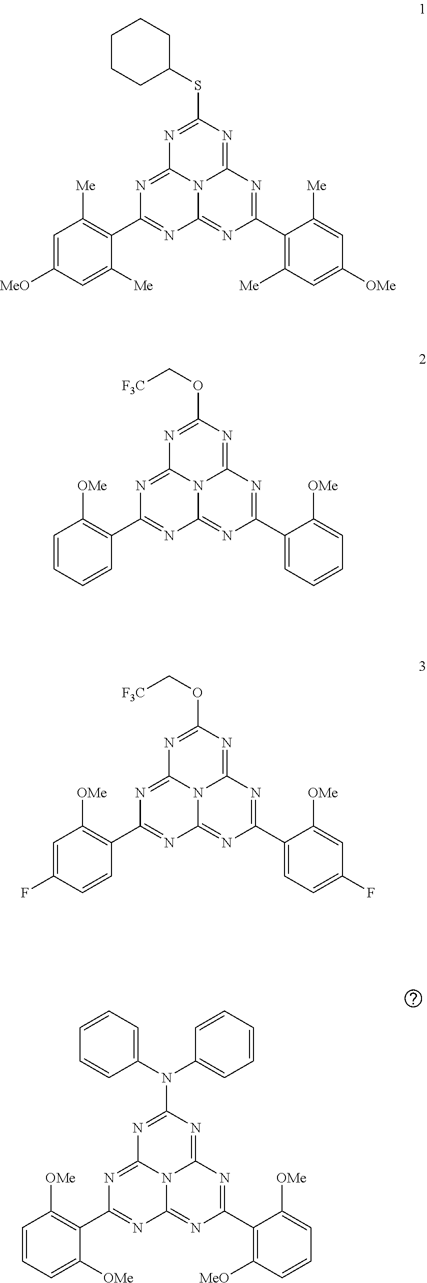 [SMILES: COc1cc(C)c(C2=NC3=NC(SC4CCCCC4)=NC4=NC(c5c(C)cc(OC)cc5C)=NC(=N2)N34)c(C)c1 ; COc1ccccc1C1=NC2=NC(OCC(F)(F)F)=NC3=NC(c4ccccc4OC)=NC(=N1)N23 ; COc1cc(F)ccc1C1=NC2=NC(OCC(F)(F)F)=NC3=NC(c4ccc(F)cc4OC)=NC(=N1)N23 ; COc1cccc(OC)c1C1=NC2=NC(c3c(OC)cccc3OC)=NC3=NC(N(c4ccccc4)c4ccccc4)=NC(=N1)N23]

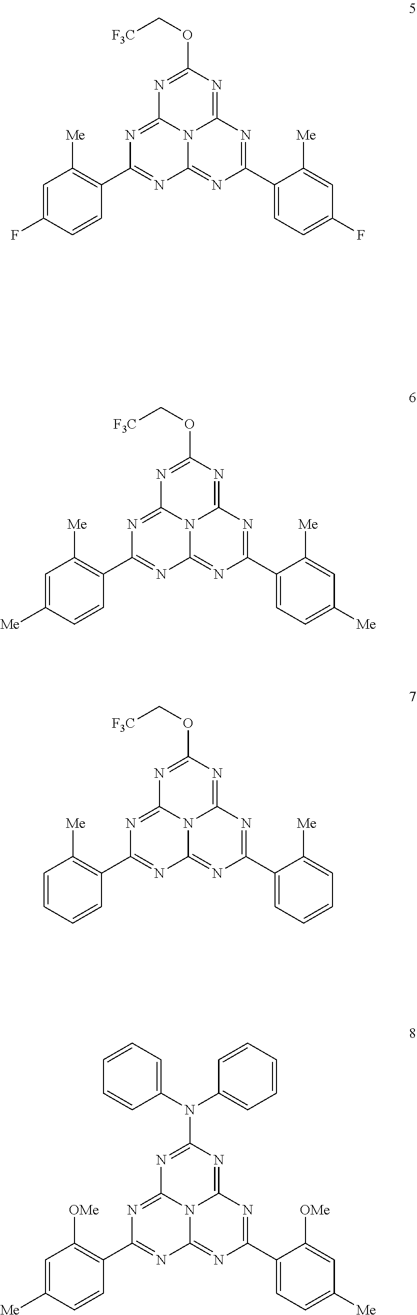 [SMILES: Cc1cc(F)ccc1C1=NC2=NC(OCC(F)(F)F)=NC3=NC(c4ccc(F)cc4C)=NC(=N1)N23 ; Cc1ccc(C2=NC3=NC(OCC(F)(F)F)=NC4=NC(c5ccc(C)cc5C)=NC(=N2)N34)c(C)c1 ; Cc1ccccc1C1=NC2=NC(OCC(F)(F)F)=NC3=NC(c4ccccc4C)=NC(=N1)N23 ; COc1cc(C)ccc1C1=NC2=NC(c3ccc(C)cc3OC)=NC3=NC(N(c4ccccc4)c4ccccc4)=NC(=N1)N23]

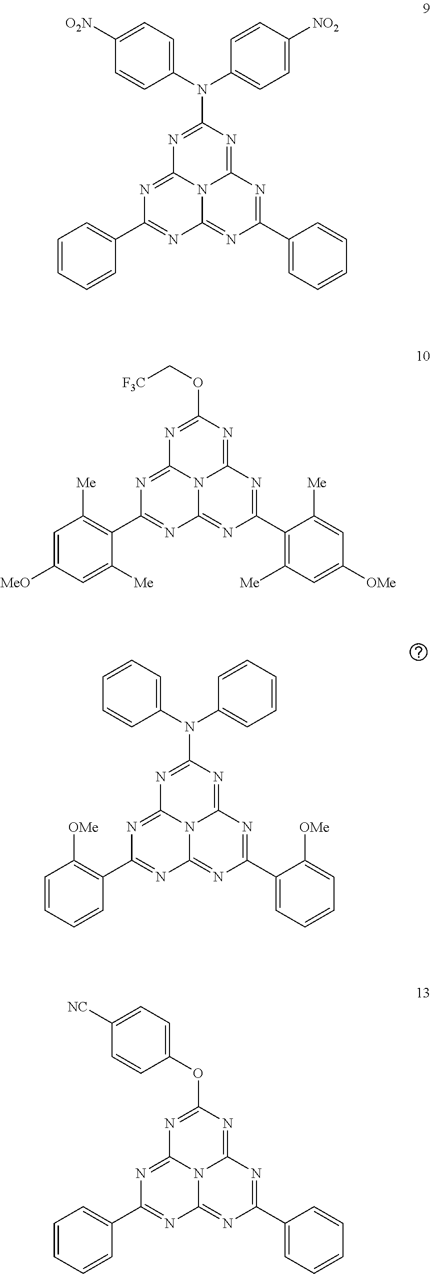 [SMILES: O=[N+]([O-])c1ccc(N(C2=NC3=NC(c4ccccc4)=NC4=NC(c5ccccc5)=NC(=N2)N43)c2ccc([N+](=O)[O-])cc2)cc1 ; COc1cc(C)c(C2=NC3=NC(OCC(F)(F)F)=NC4=NC(c5c(C)cc(OC)cc5C)=NC(=N2)N34)c(C)c1 ; COc1ccccc1C1=NC2=NC(c3ccccc3OC)=NC3=NC(N(c4ccccc4)c4ccccc4)=NC(=N1)N23 ; N#Cc1ccc(OC2=NC3=NC(c4ccccc4)=NC4=NC(c5ccccc5)=NC(=N2)N34)cc1]

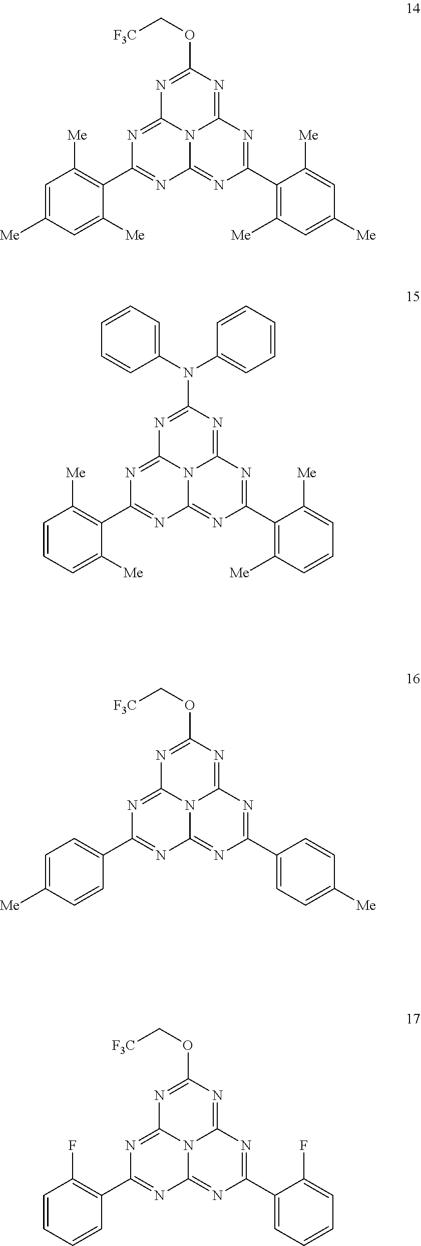 [SMILES: Cc1cc(C)c(C2=NC3=NC(OCC(F)(F)F)=NC4=NC(c5c(C)cc(C)cc5C)=NC(=N2)N34)c(C)c1 ; Cc1cccc(C)c1C1=NC2=NC(c3c(C)cccc3C)=NC3=NC(N(c4ccccc4)c4ccccc4)=NC(=N1)N23 ; Cc1ccc(C2=NC3=NC(OCC(F)(F)F)=NC4=NC(c5ccc(C)cc5)=NC(=N2)N34)cc1 ; Fc1ccccc1C1=NC2=NC(OCC(F)(F)F)=NC3=NC(c4ccccc4F)=NC(=N1)N23]

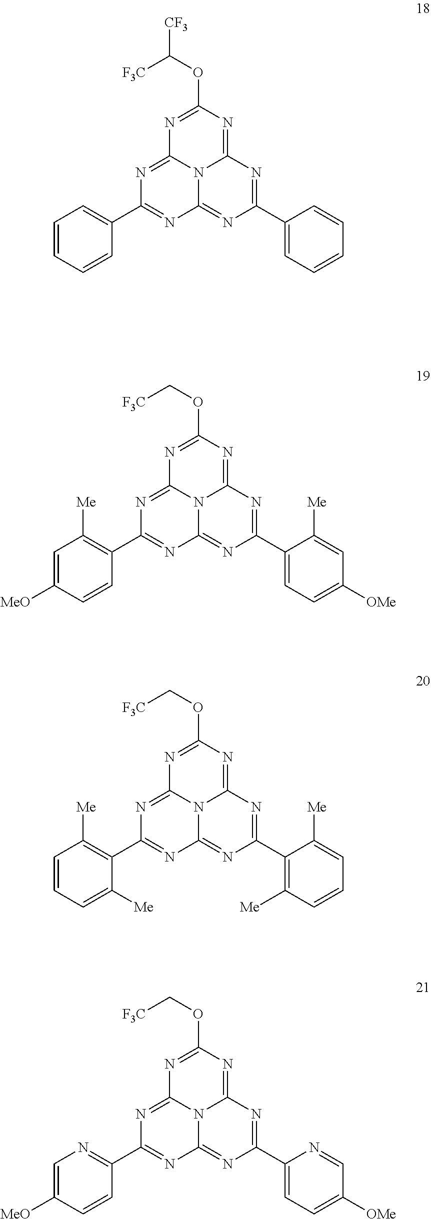 [SMILES: FC(F)(F)C(OC1=NC2=NC(c3ccccc3)=NC3=NC(c4ccccc4)=NC(=N1)N23)C(F)(F)F ; COc1ccc(C2=NC3=NC(OCC(F)(F)F)=NC4=NC(c5ccc(OC)cc5C)=NC(=N2)N34)c(C)c1 ; Cc1cccc(C)c1C1=NC2=NC(OCC(F)(F)F)=NC3=NC(c4c(C)cccc4C)=NC(=N1)N23 ; COc1ccc(C2=NC3=NC(OCC(F)(F)F)=NC4=NC(c5ccc(OC)cn5)=NC(=N2)N34)nc1]

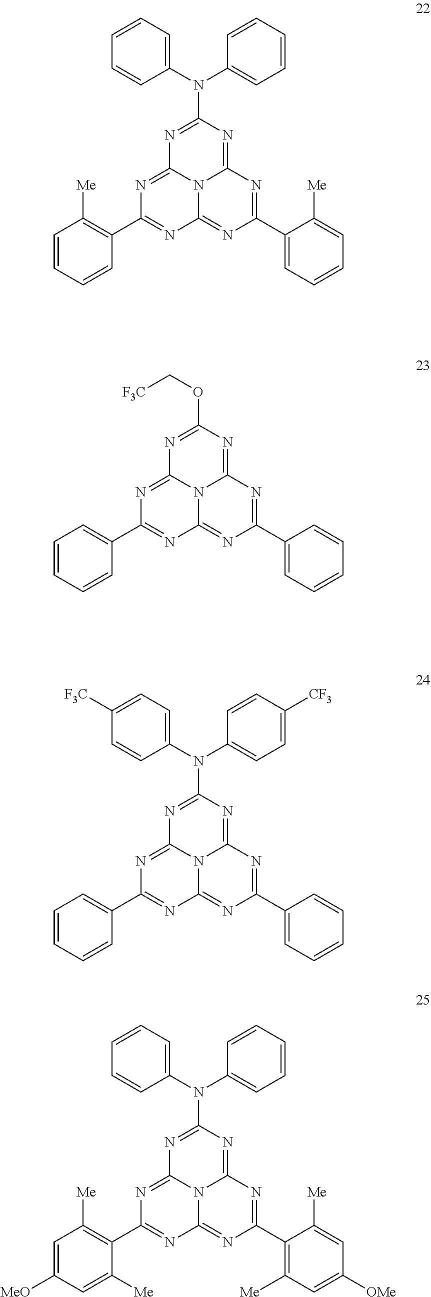 [SMILES: Cc1ccccc1C1=NC2=NC(c3ccccc3C)=NC3=NC(N(c4ccccc4)c4ccccc4)=NC(=N1)N23 ; FC(F)(F)COC1=NC2=NC(c3ccccc3)=NC3=NC(c4ccccc4)=NC(=N1)N23 ; FC(F)(F)c1ccc(N(C2=NC3=NC(c4ccccc4)=NC4=NC(c5ccccc5)=NC(=N2)N43)c2ccc(C(F)(F)F)cc2)cc1 ; COc1cc(C)c(C2=NC3=NC(c4c(C)cc(OC)cc4C)=NC4=NC(N(c5ccccc5)c5ccccc5)=NC(=N2)N34)c(C)c1]

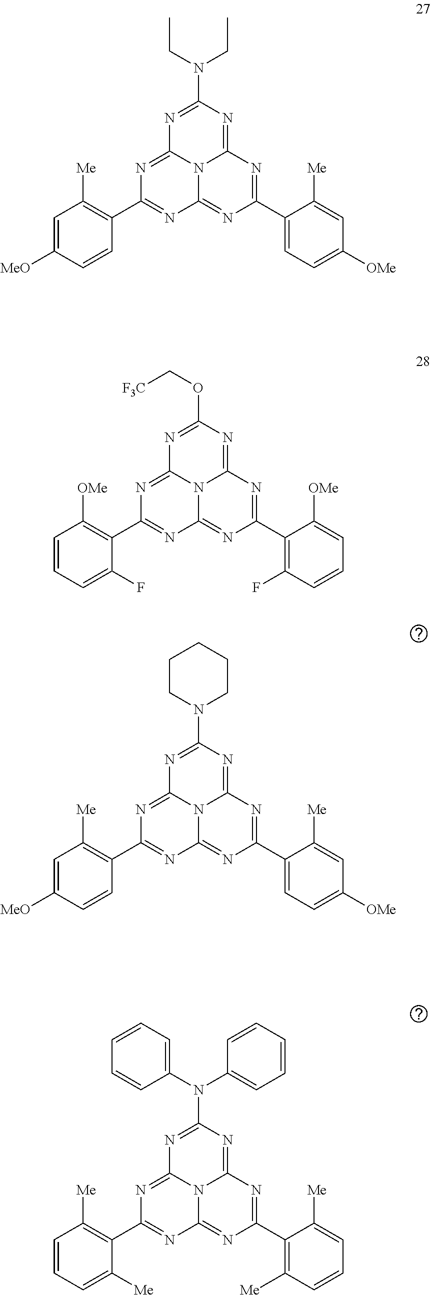 [SMILES: CCN(CC)C1=NC2=NC(c3ccc(OC)cc3C)=NC3=NC(c4ccc(OC)cc4C)=NC(=N1)N32 ; COc1cccc(F)c1C1=NC2=NC(OCC(F)(F)F)=NC3=NC(c4c(F)cccc4OC)=NC(=N1)N23 ; COc1ccc(C2=NC3=NC(c4ccc(OC)cc4C)=NC4=NC(N5CCCCC5)=NC(=N2)N34)c(C)c1 ; Cc1cccc(C)c1C1=NC2=NC(c3c(C)cccc3C)=NC3=NC(N(c4ccccc4)c4ccccc4)=NC(=N1)N23]

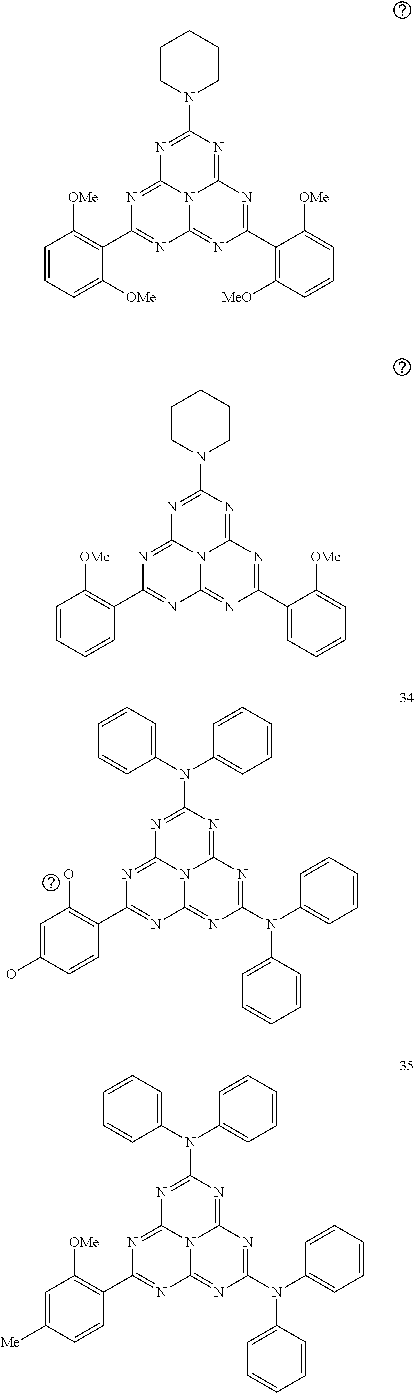 [SMILES: COc1cccc(OC)c1C1=NC2=NC(c3c(OC)cccc3OC)=NC3=NC(N4CCCCC4)=NC(=N1)N23 ; COc1ccccc1C1=NC2=NC(c3ccccc3OC)=NC3=NC(N4CCCCC4)=NC(=N1)N23 ; Oc1ccc(C2=NC3=NC(N(c4ccccc4)c4ccccc4)=NC4=NC(N(c5ccccc5)c5ccccc5)=NC(=N2)N34)c(O)c1 ; COc1cc(C)ccc1C1=NC2=NC(N(c3ccccc3)c3ccccc3)=NC3=NC(N(c4ccccc4)c4ccccc4)=NC(=N1)N23]

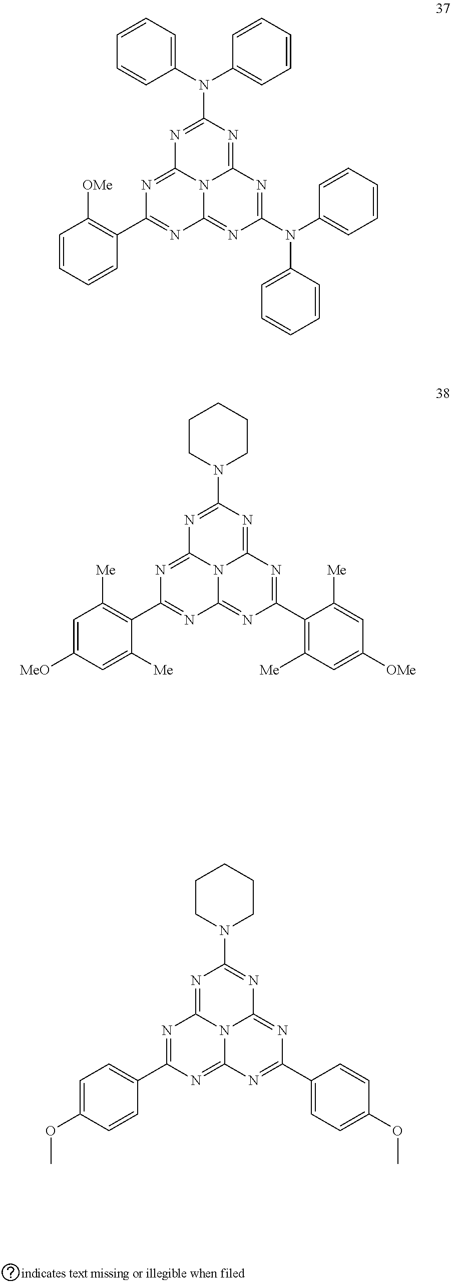 [SMILES: COc1ccccc1C1=NC2=NC(N(c3ccccc3)c3ccccc3)=NC3=NC(N(c4ccccc4)c4ccccc4)=NC(=N1)N23 ; COc1cc(C)c(C2=NC3=NC(c4c(C)cc(OC)cc4C)=NC4=NC(N5CCCCC5)=NC(=N2)N34)c(C)c1 ; COc1ccc(C2=NC3=NC(c4ccc(OC)cc4)=NC4=NC(N5CCCCC5)=NC(=N2)N34)cc1]

Among the above, organic compounds with the numbers 1, 2, 3, 4 (11), 6, 16, 23, 25, 27, and 29 (33) may be more preferable.

### Example of Method of Synthesizing Organic Compound Represented by Formula (1) or Formula (2)

A method of synthesizing the organic compound is not particularly limited. For example, the organic compound can be synthesized by causing a compound (precursor compound) in which R1 through R3 in Formula (1) or Formula (2) are halogen groups to react with compounds corresponding to R1, R2, and R3 in the presence of a Lewis acid catalyst (e.g., aluminum chloride). In a case where two or more compounds are used as the compounds corresponding to R1, R2, and R3, different R1, R2, and R3 may be introduced by appropriately adjusting the amounts of these compounds used, the timing of addition to the reaction system, and other reaction conditions. For details of the synthesis method, Examples described below may also be referred to.

<Application and the Like of Present Organic Compound>

The present organic compound can be suitably used, for example, as a luminescent material for a luminescent layer of an organic light emitting element or an organic light emitting device. The present organic compound may form a luminescent layer by the compound alone, or may form a luminescent layer as a composition (sometimes referred to as “luminescent composition”) formed by mixing the present organic compound with another compound. An organic light emitting element and an organic light emitting device in each of which the present organic compound is contained in a luminescent layer are also encompassed within the scope of the present invention.

The luminescent layer often contains a host compound and a dopant compound. The dopant compound is sometimes referred to as a guest compound. The host compound is responsible for electric charge (electron and positive hole) transport. The dopant compound is responsible for luminescence. The present organic compound may be used as a host compound or a dopant compound in the luminescent layer. In particular, among the present organic compounds, those in which the energy gap ΔE_{ST} satisfies the relation of −0.20 eV≤ΔE_{ST}<0.0090 eV can be used as any of the host compound and the dopant compound. Among the present organic compounds, those in which the energy gap ΔE_{ST} satisfies the relation of ΔE_{ST}<−0.20 eV are preferably used as a host compound.

A technique used in preparation of the luminescent layer is not limited. As a method of preparing a luminescent layer, for example, a vacuum deposition method may be employed, or a coating method may be employed. Examples of the coating method include an inkjet method, a gravure printing method, and a nozzle coating method. A substrate included in the organic light emitting device may be a hard substrate represented by glass, or a flexible substrate represented by a resin, as long as the substrate has a light-transmitting property.

### Examples

### First Example

The following description will discuss an organic compound A which is a first example of the present invention. The organic compound A is a heptazine derivative represented by Formula (4) below. That is, in the organic compound A, a mother nucleus is heptazine, three substituents R1, R2, and R3 are such that R1 is 1-piperidinyl group (i.e., —N—(R32)R33 indicated in Formula (4), and —R32 and —R33 are bonded to each other to form a ring structure), and R2 and R3 are both 4-methoxyphenyl groups. That is, the three substituents are constituted by two types of substituents.

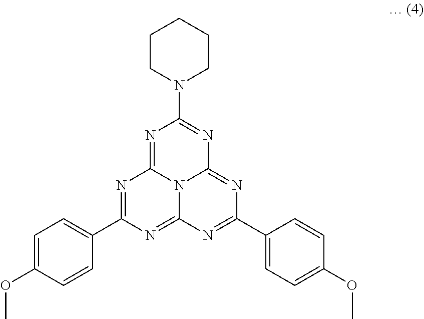 [SMILES: COc1ccc(C2=NC3=NC(c4ccc(OC)cc4)=NC4=NC(N5CCCCC5)=NC(=N2)N34)cc1]

<Calculation of Energy Gap ΔE_{ST} and Oscillator Strength f>

(TDDFT Calculation)

Structural optimization of a lowest singlet excitation mode S₁ and a lowest triplet excitation state T₁ of the organic compound A was carried out using TDDFT calculation, and an energy gap ΔE_{ST} and an oscillator strength f of the organic compound A were calculated. TDDFT calculation included in Gaussian 16 was used for the TDDFT calculation, ωB97X-D was used for a functional, and 6-31G(d) was used for a basis function.

(ADC(2) Calculation)

In a most stable structure in the lowest triplet excitation state T₁ of the organic compound A obtained by the above described TDDFT calculation, the energy gap ΔE_{ST} and the oscillator strength f of the organic compound A were calculated using ADC(2) calculation. ADC(2) calculation included in Q-Chem5.2 was used for the ADC(2) calculation, and 6-31 G(d) was used for a basis function.

The energy gap ΔE_{ST} and the oscillator strength f of the organic compound A calculated by the TDDFT calculation and the ADC(2) calculation are indicated in Table 1. The energy gap ΔE_{ST} and the oscillator strength f of the organic compound A calculated using the ADC(2) calculation, which can take into account two-electron excitation, are ΔE_{ST}=−0.35 eV and f=0.017, respectively. That is, the organic compound A was expected to exhibit a negative energy gap ΔE_{ST} and a relatively large oscillator strength f.

TABLE 1

| Calculation | ΔE_{ST} | |
|---|---|---|
| method | (eV) | f |
| TDDFT | 0.27 | 0.018 |
| ADC (2) | −0.35 | 0.017 |

<Synthesis Scheme>

The organic compound A was obtained by a synthesis scheme described below. That is, in an argon atmosphere, AlCl₃ (1.83 mmol) was added to a dichloromethane solution (5 ml) of Anisole (2.5 mmol) at 0° C. The resulting mixture was left at that temperature for 40 minutes, and trichloroheptazine (0.83 mmol) was added at 0° C. 10 minutes later, the temperature was raised to room temperature, and the mixture was rotated overnight. 20 hours later, reflux was initiated, and the reflux was continued for 4 hours. When the temperature was returned to room temperature, 0.5 ml (excessive amount) of piperidine was added. 1 hour later, water was added for quenching. By column purification (1% AcOEt/DCM→15% AcOEt/DCM), a yellow-white organic compound A was isolated. In this example, a yield of the organic compound A was 15%.

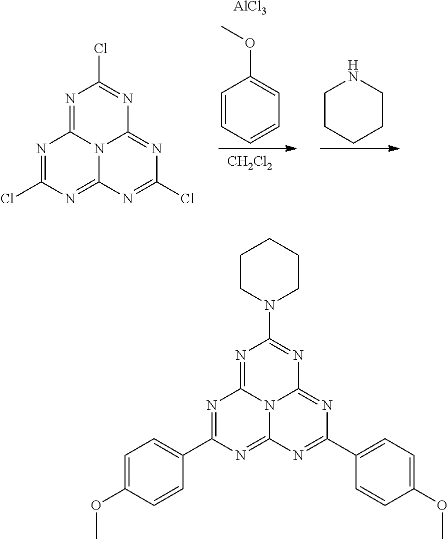

<Luminescence Property>

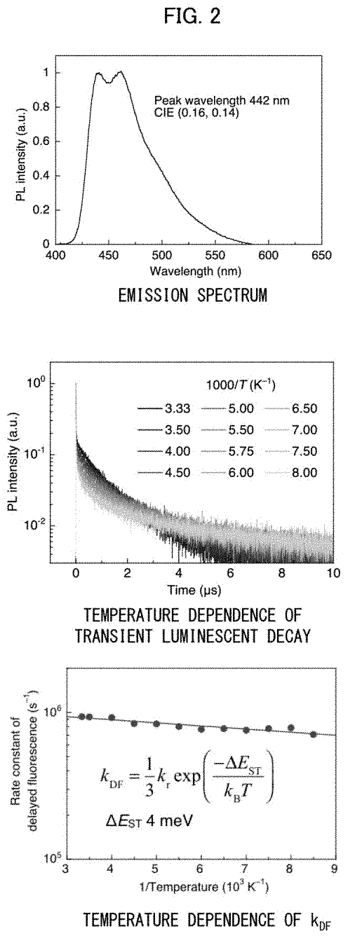

For a mixed thin film of the organic compound A thus synthesized and 2,8-bis(diphenylphosphoryl)dibenzo[b,d]furan (PPF), (1) an emission spectrum was measured using a spectrophotofluorometer Fluoromax-4 manufactured by HORIBA, (2) a luminescent quantum yield was measured using an integrating sphere C9920 manufactured by Hamamatsu Photonics K.K., and (3) a luminescence lifetime τ of delayed fluorescence was measured using Fluorolog-3 manufactured by HORIBA. In the mixed thin film of this example, a concentration of the organic compound A was 5 wt %. The upper part, middle part, and lower part in FIG. 2 are graphs indicating an emission spectrum, temperature dependence of transient luminescent decay, and temperature dependence of a rate constant k_{DF} of delayed fluorescence in the mixed thin film of this example.

Measurements of (1), (2), and (3) described above were carried out under an inert nitrogen atmosphere. The measurement of (3) described above was carried out while changing the temperature using a cryostat CoolSpeK manufactured by UNISOKU. The temperature dependence of the obtained luminescence lifetime T was analyzed by a mathematical formula (3) assuming a thermal equilibrium between the lowest singlet excitation mode S₁ and the lowest triplet excitation state T₁, and experimental values of the energy gap ΔE_{ST} and the radiative decay rate constant kᵣ were estimated. In the mathematical formula (3), k_{B} is a Boltzmann constant, T is an absolute temperature, and k_{DF} is a rate constant of delayed fluorescence.

$${τ}^{- 1} = {k}_{DF} = \frac{1}{3} {k}_{r} exp ( \frac{- Δ {E}_{ST}}{{k}_{B} T} ) \qquad ( 3 )$$

The mixed thin film of this example exhibited blue luminescence (CIE 0.16, 0.14) with a maximum emission wavelength of 442 nm (see the upper part in FIG. 2). The mixed thin film of this example also exhibited a high luminescent quantum yield of 85%, a short luminescence lifetime τ of 1066 ns, and a radiative decay rate constant kᵣ of 1.2×10⁸s⁻¹. From the temperature dependence of the luminescence lifetime τ, the energy gap ΔE_{ST} was estimated to be ΔE_{ST}=0.004 eV (see the middle and lower parts in FIG. 2). The oscillator strength f, in which degeneracy estimated from the radiative decay rate constant kᵣ using a mathematical formula (4) was not taken into account, was f=0.35.

$$f = \frac{{ε}_{0} {m}_{e} {ck}_{r}}{2 π {e}^{2} {\tilde{𝓋}}^{2}} \qquad ( 4 )$$

In the mathematical formula (4), e is an elementary charge, me is an electron mass, ε₀ is a dielectric constant in vacuo, c is a light velocity, and
\tilde{V}
   is a wave number of luminescence.

### First Referential Example

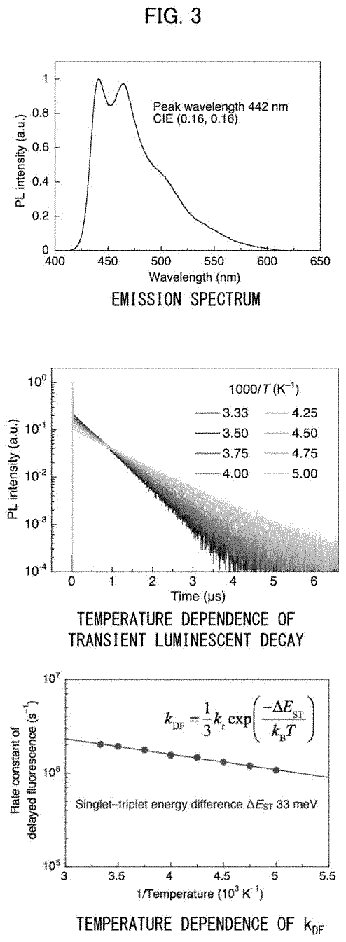

A toluene solution of the organic compound A synthesized using the synthesis scheme described in the first example is regarded as a first referential example. In the toluene solution of this referential example, a concentration of the organic compound A was 8×10⁻⁵ M. For the toluene solution in this referential example also, (1) an emission spectrum was measured using a spectrophotofluorometer Fluoromax-4 manufactured by HORIBA, (2) a luminescent quantum yield was measured using an integrating sphere C9920 manufactured by Hamamatsu Photonics K.K., and (3) a luminescence lifetime τ of delayed fluorescence was measured using Fluorolog-3 manufactured by HORIBA, as with the first example. The upper part, middle part, and lower part in FIG. 3 are graphs indicating an emission spectrum, temperature dependence of transient luminescent decay, and temperature dependence of a rate constant k_{DF} of delayed fluorescence in the toluene solution of this referential example.

The toluene solution of this referential example exhibited blue luminescence (CIE 0.16, 0.16) with a maximum emission wavelength of 442 nm (see the upper part in FIG. 3). The toluene solution of this referential example also exhibited a high luminescent quantum yield of 75%, and a short luminescence lifetime τ of 588 ns. From the temperature dependence of the luminescence lifetime τ, the energy gap ΔE_{ST} was estimated to be ΔE_{ST}=0.033 eV, and the radiative decay rate constant kᵣ, was estimated to be kᵣ=2.2×10⁷s⁻¹ (see the middle and lower parts in FIG. 3).

(Second example group) The following description will discuss organic compounds 1 through 38 which are a second example group of the present invention. The organic compounds 1 through 38 are respectively 38 organic compounds described above as particularly preferable examples of the present organic compound.

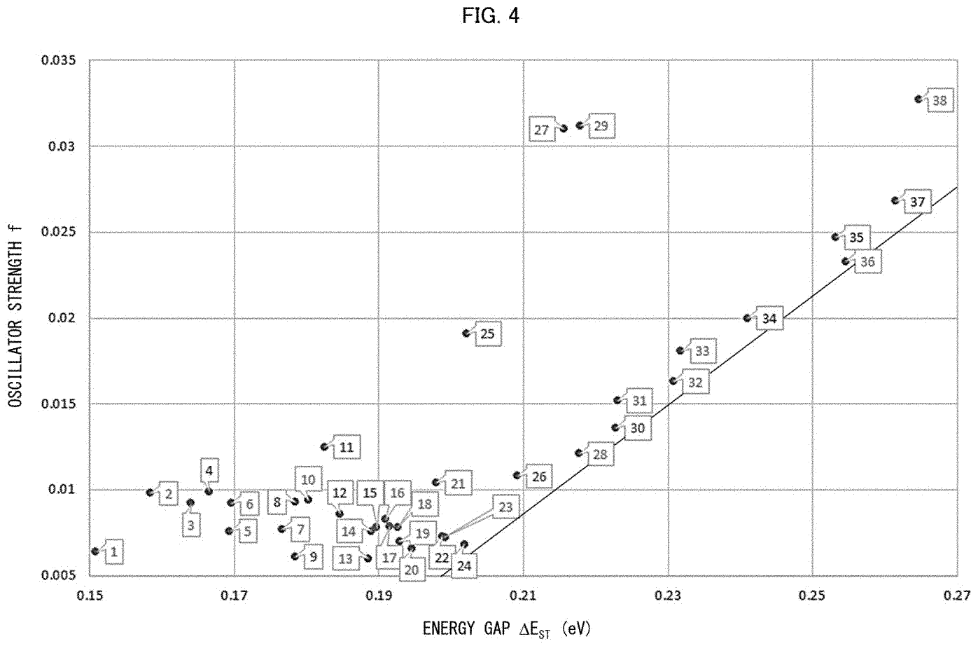

As with the first example, structural optimization of a lowest singlet excitation mode S₁ and a lowest triplet excitation state T₁ of each of the organic compounds 1 through 38 was carried out using TDDFT calculation, and an energy gap ΔE_{ST} and an oscillator strength f of each of the organic compounds 1 through 38 were calculated. FIG. 4 is a graph indicating a correlation between an energy gap ΔE_{ST} and an oscillator strength f in each of the organic compounds 1 through 38. The solid line in FIG. 4 indicates a function f_{(ΔEST)} represented by f_{(ΔEST)}=(ΔE_{ST}−0.18)×0.3. That is, each of the organic compounds 1 through 38 satisfies a relational expression off (ΔE_{ST}−0.18)×0.3.

The energy gap ΔE_{ST} of the organic compound A of the first example was ΔE_{ST}=0.27 eV when TDDFT calculation was used. Meanwhile, when calculated from the luminescence property of the synthesized organic compound A, the energy gap ΔE_{ST} was ΔE_{ST}=0.0040 eV. That is, it has been found that the energy gap ΔE_{ST} calculated from the luminescence property is shifted to be smaller, as compared with the energy gap ΔE_{ST} obtained using TDDFT calculation.

Based on the result of the first example described above, the organic compounds 1 through 38 in which the energy gap ΔE_{ST} and the oscillator strength f obtained by using the TDDFT calculation satisfy the relational expression of f≥(ΔE_{ST}−0.18)×0.3 in a space derived from the energy gap ΔE_{ST} and the oscillator strength f are encompassed in the scope of the present invention.

### First Comparative Example

The following description will discuss an organic compound B disclosed in Non-patent Literature 2. The organic compound B is a heptazine derivative represented by Formula (5) below. That is, in the organic compound B, a mother nucleus is heptazine, and three substituents R1, R2, and R3 are all 4-methoxyphenyl groups.

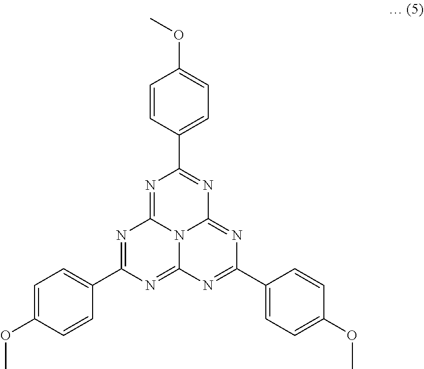 [SMILES: COc1ccc(C2=NC3=NC(c4ccc(OC)cc4)=NC4=NC(c5ccc(OC)cc5)=NC(=N2)N34)cc1]

For the organic compound B, the energy gap ΔE_{ST} and an oscillator strength f calculated using ADC(2) calculation were ΔE_{ST}=−0.250 eV and f=0.0000050.

For the organic compound B, a toluene solution was prepared, and (1) an emission spectrum was measured using a spectrophotofluorometer Fluoromax-4 manufactured by HORIBA, (2) a luminescent quantum yield was measured using an integrating sphere C9920 manufactured by Hamamatsu Photonics K.K., and (3) a luminescence lifetime τ was measured using Fluorolog-3 manufactured by HORIBA, as with the first example. As a result, the radiative decay rate constant kᵣ and the oscillator strength f were kᵣ=1.0×10⁶s⁻¹ and f=0.0039, respectively. It was found that the organic compound B did not exhibit delayed fluorescence. Therefore, the energy gap ΔE_{ST} could not be evaluated for the organic compound B.

As described above, the organic compound B does not exhibit delayed fluorescence. Therefore, the energy gap ΔE_{ST} cannot be evaluated, and the organic compound B is not encompassed in the scope of the present invention. The organic compound B has a low intensity of fluorescence due to an extremely small oscillator strength. Therefore, it is difficult to use the organic compound B as a luminescent material for a display.

### Third Example Group

The following description will discuss organic compounds pX-Y which are a third example group of the present invention.

<Naming Rule of Organic Compounds>

In the organic compounds pX-Y, X and Y are each an integer of 1 or more and 186 or less, and correspond to the numbers of the 186 types of substituents exemplified in the section of (Examples of R1 through R3). The organic compounds pX-Y employ, in the structure of Formula (1) below, a substituent specified by X which is common as R2 and R3 and a substituent specified by Y as R1.

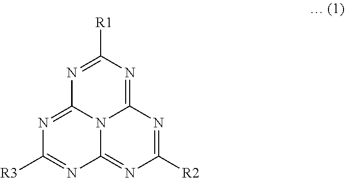

For example, an organic compound p37-151 is represented by Formula (6) below.

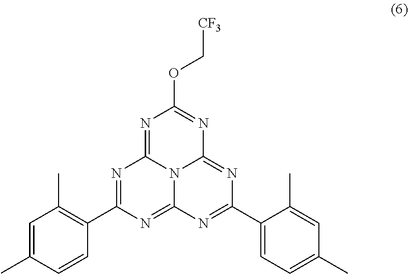

<Screening Calculation>

In the organic compounds pX-Y, R2 and R3 are selected from the 186 substituents, and R1 is similarly selected from the 186 substituents. Therefore, the organic compounds pX-Y are a group of a total of 34596 organic compounds.

Structural optimization of T₁ was carried out for these 34596 organic compounds pX-Y by Unrestricted DFT included in Gaussian 16. LC-BLYP was used for a functional, 0.18 Bohr⁻¹ was used for a domain decomposition parameter, and 6-31 G was used for a basis function. The resulting T₁ optimized structure was used to calculate an energy gap ΔE_{ST} and an oscillator strength f by TDDFT calculation. LC-BLYP was used for a functional, 0.18 Bohr⁻¹ was used for a domain decomposition parameter, and 6-31 G(d) was used for a basis function. Hereinafter, this calculation is referred to as “screening calculation”.

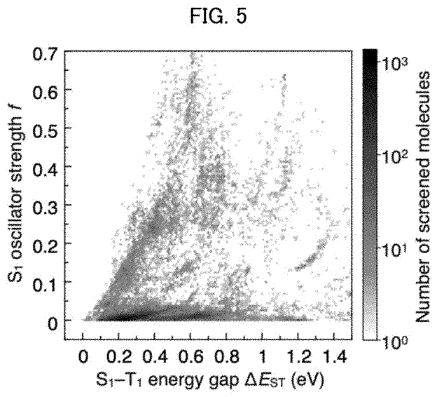

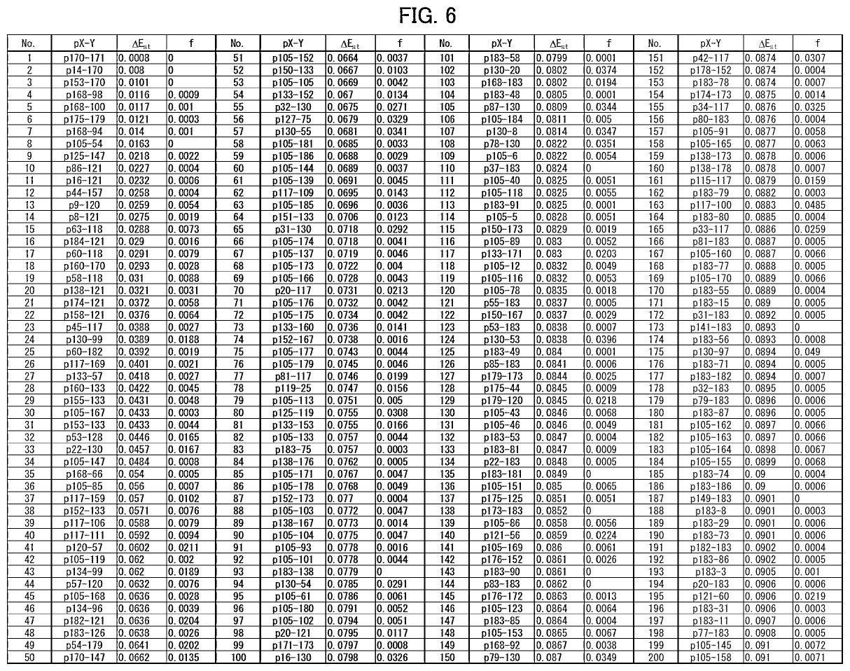

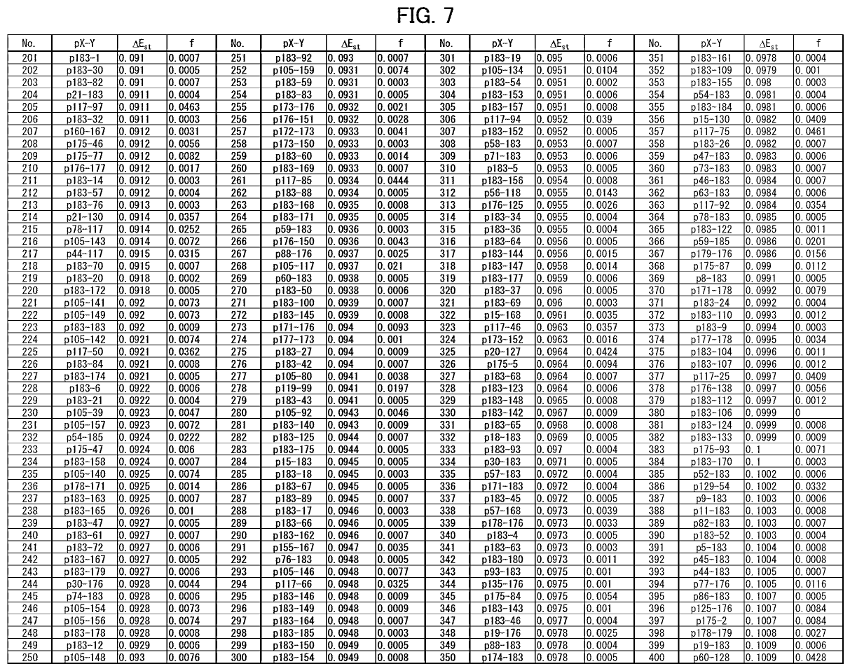

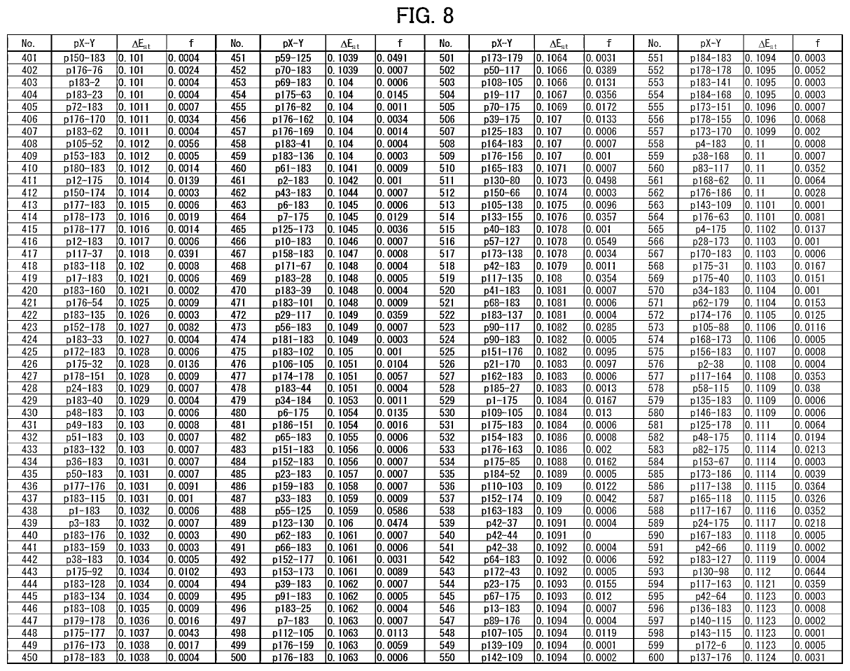

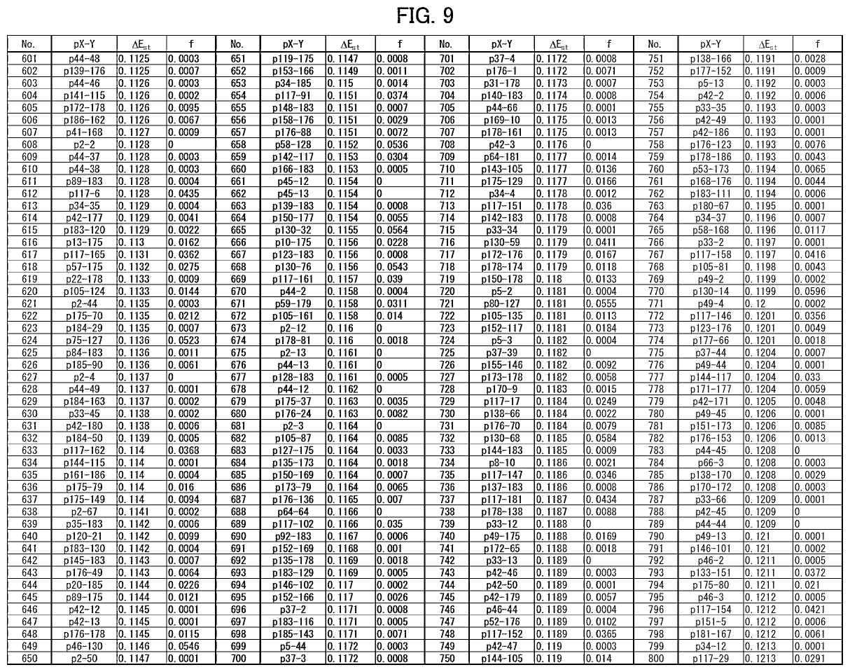

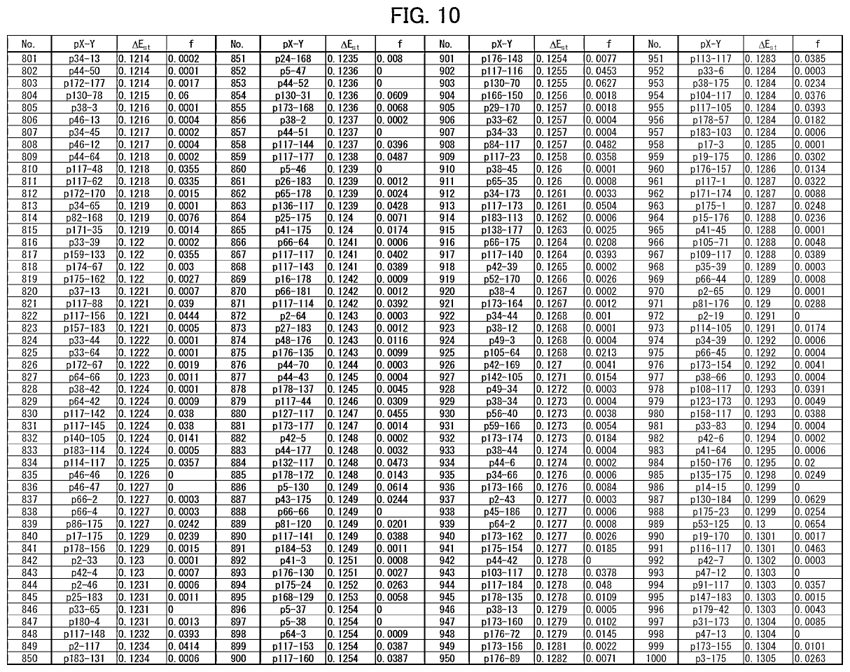

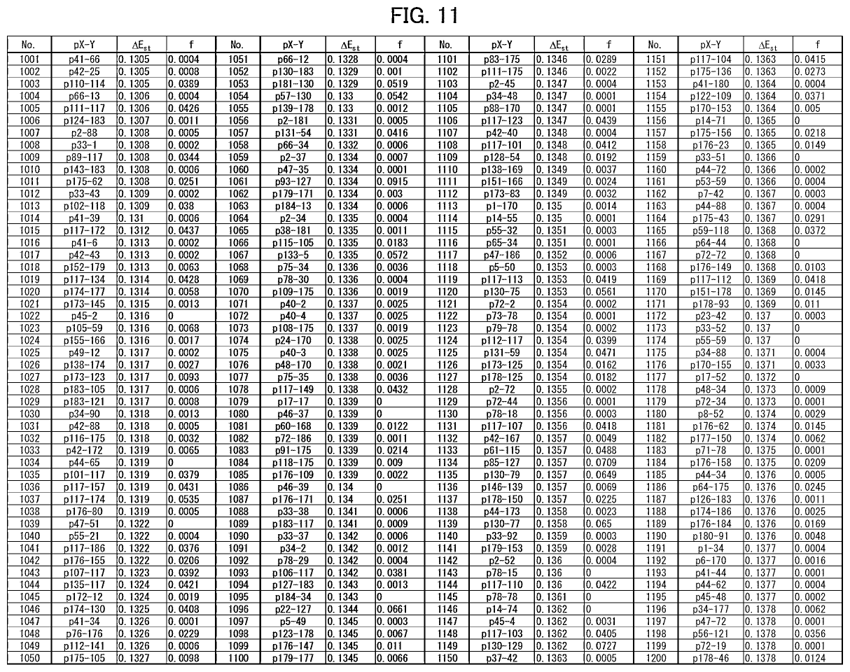

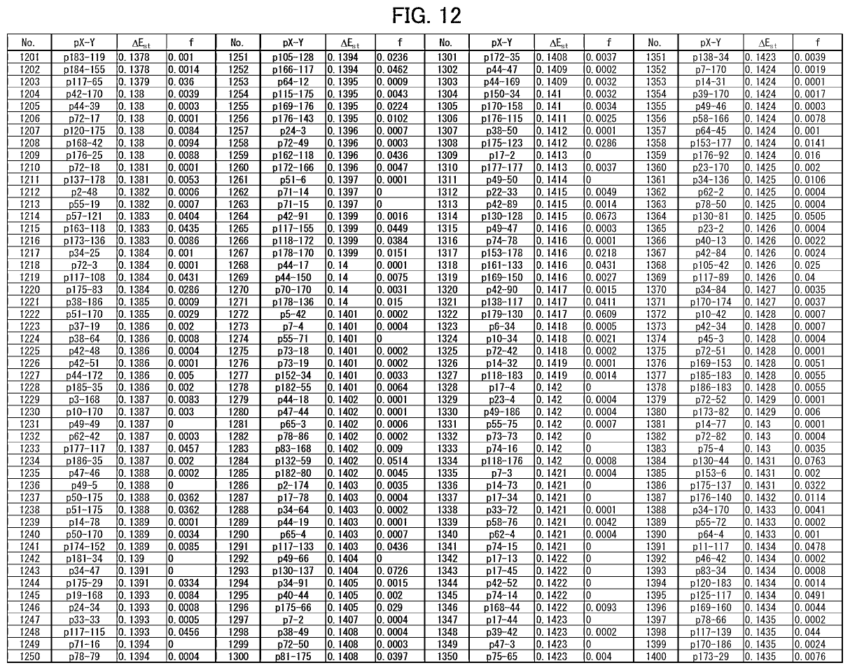

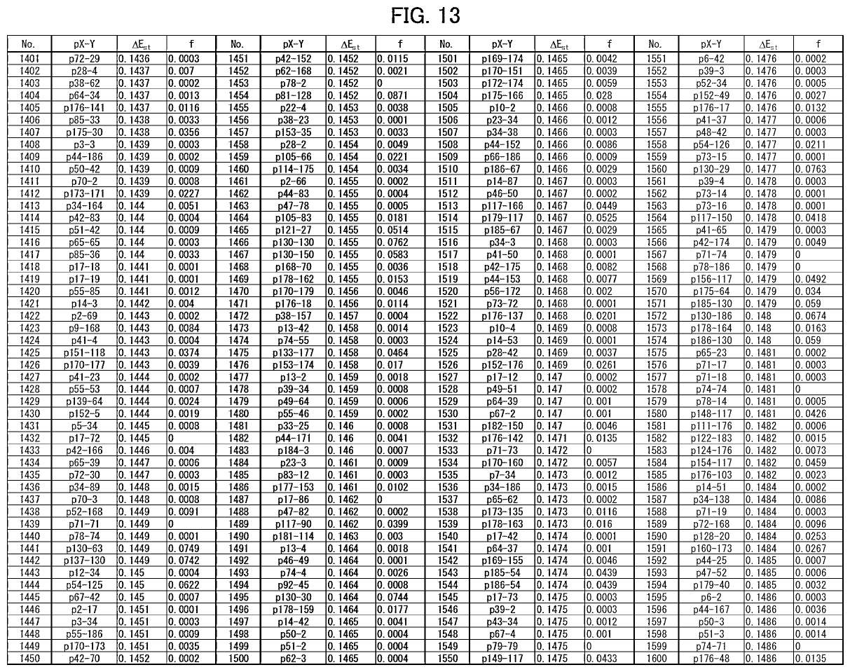

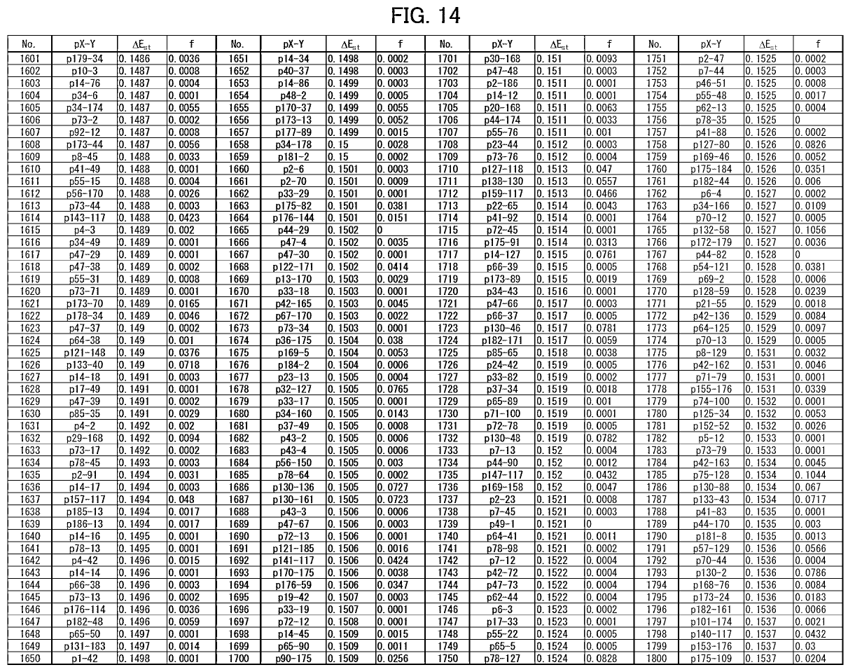

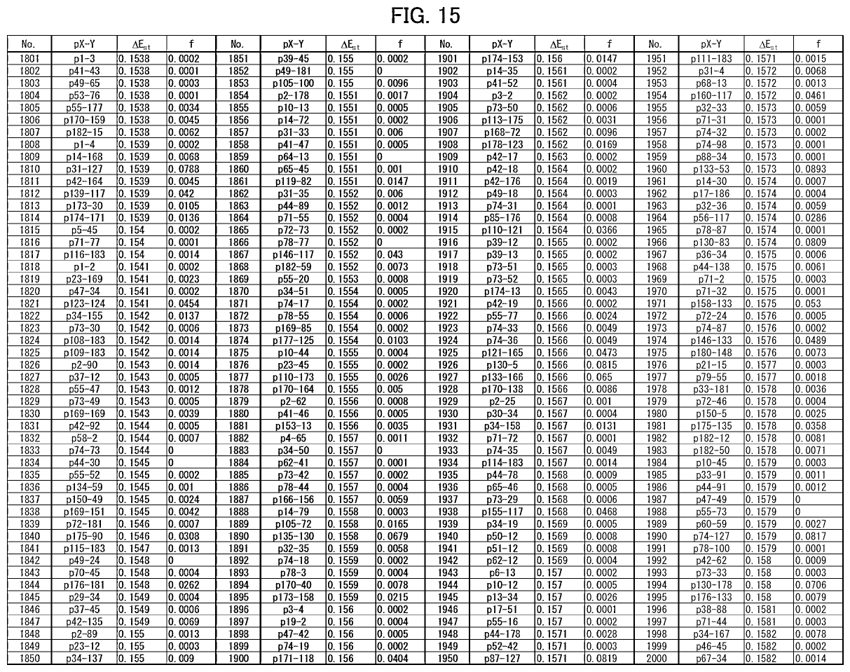

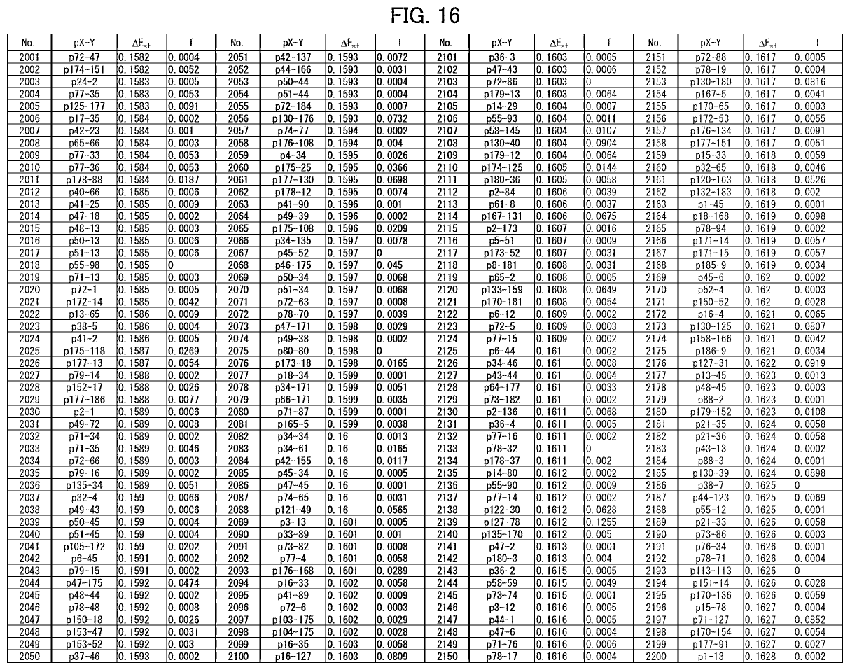

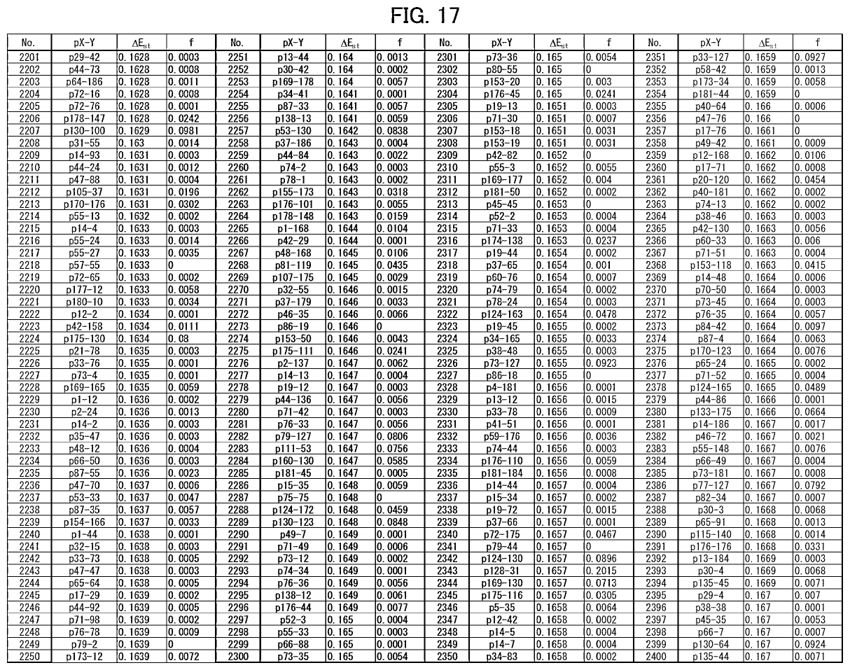

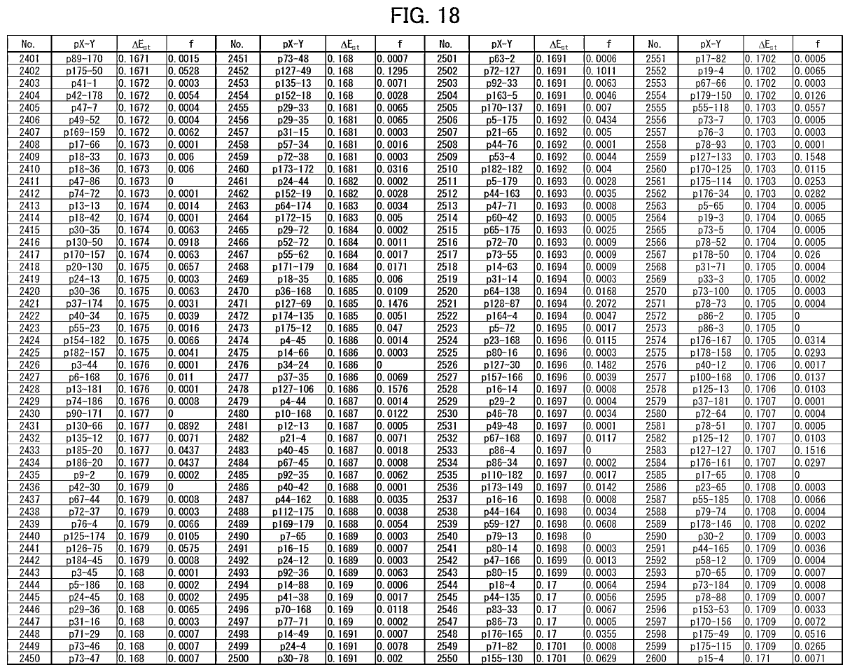

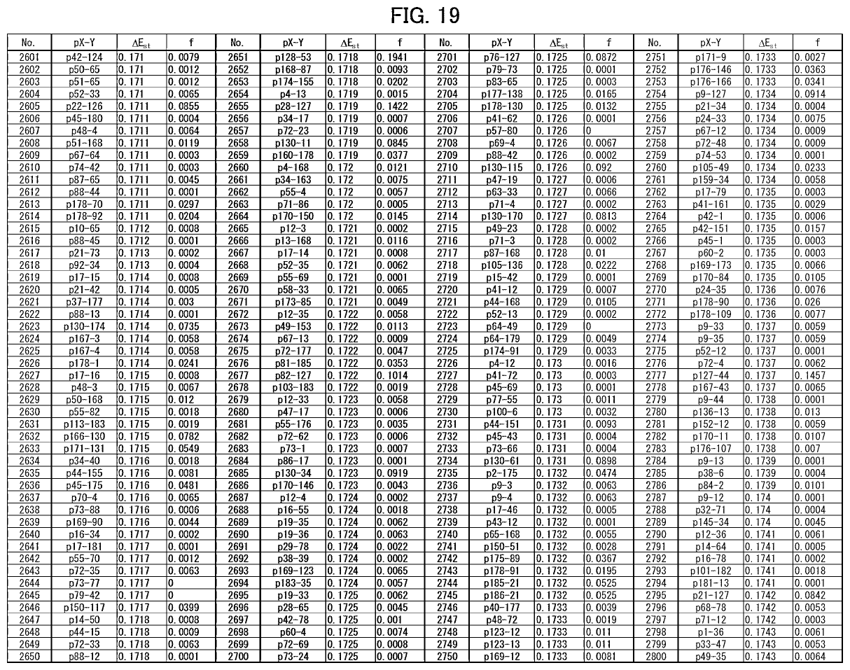

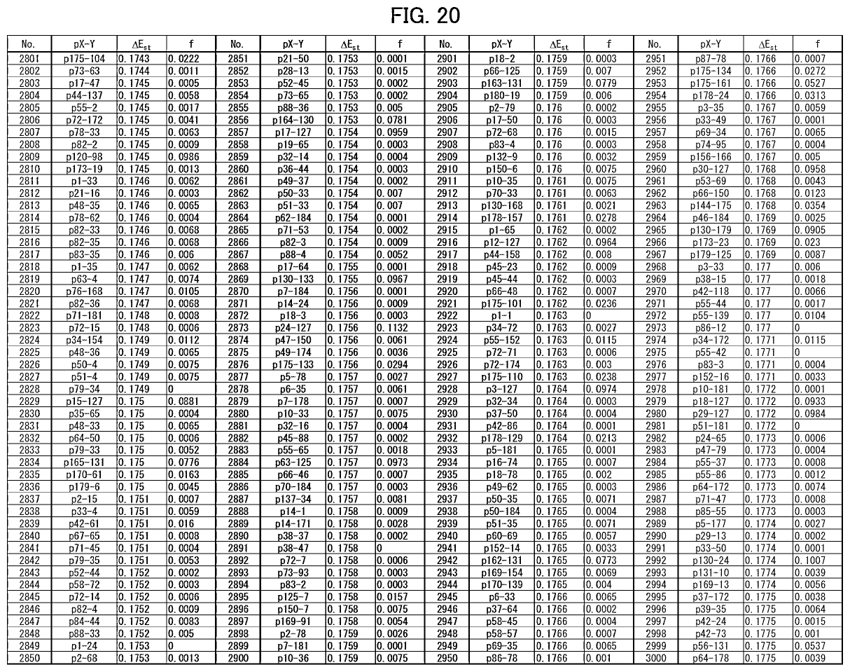

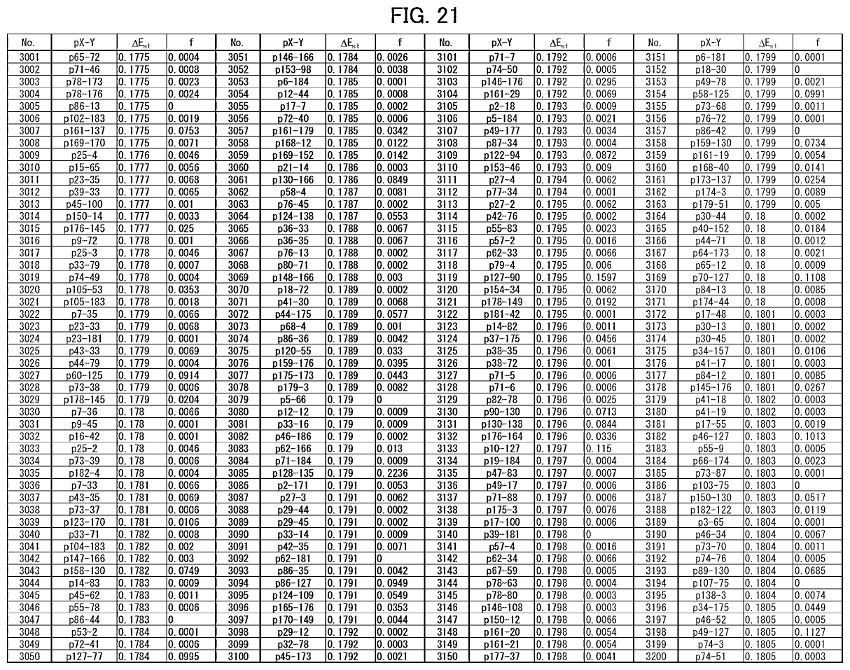

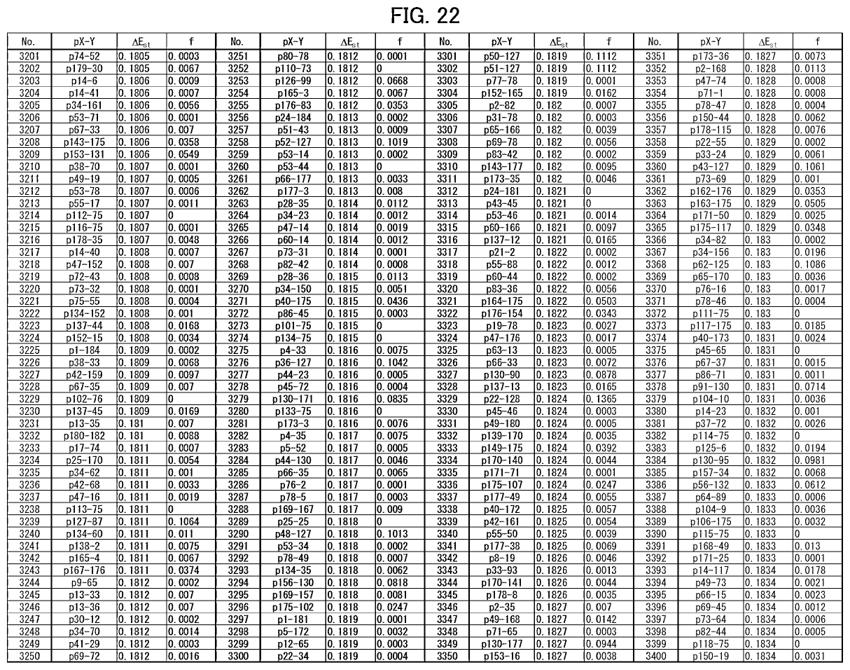

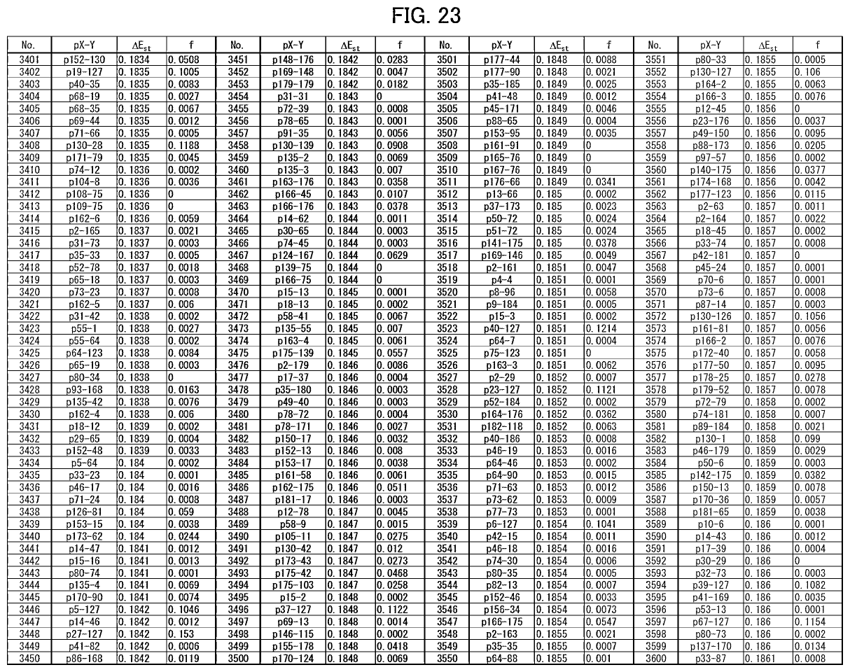

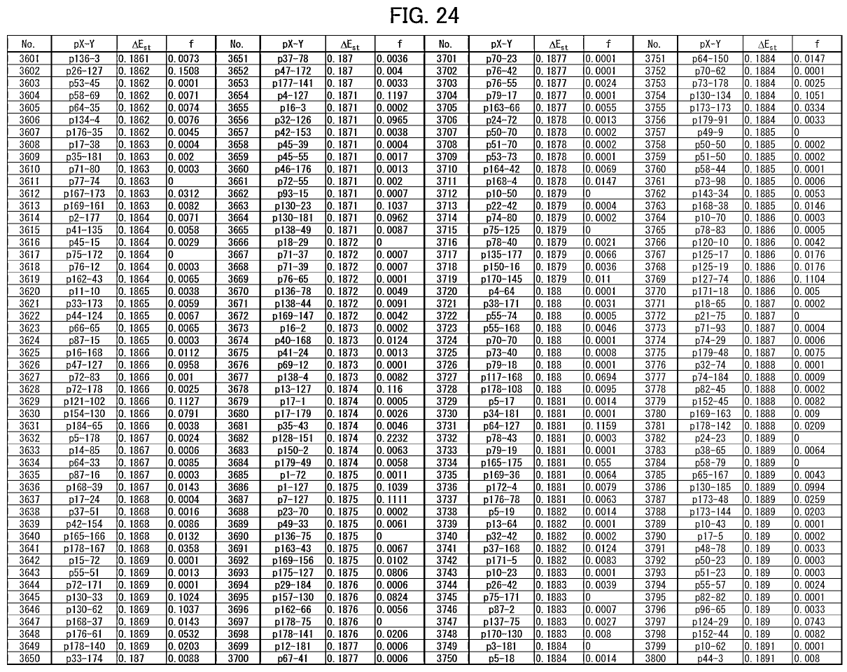

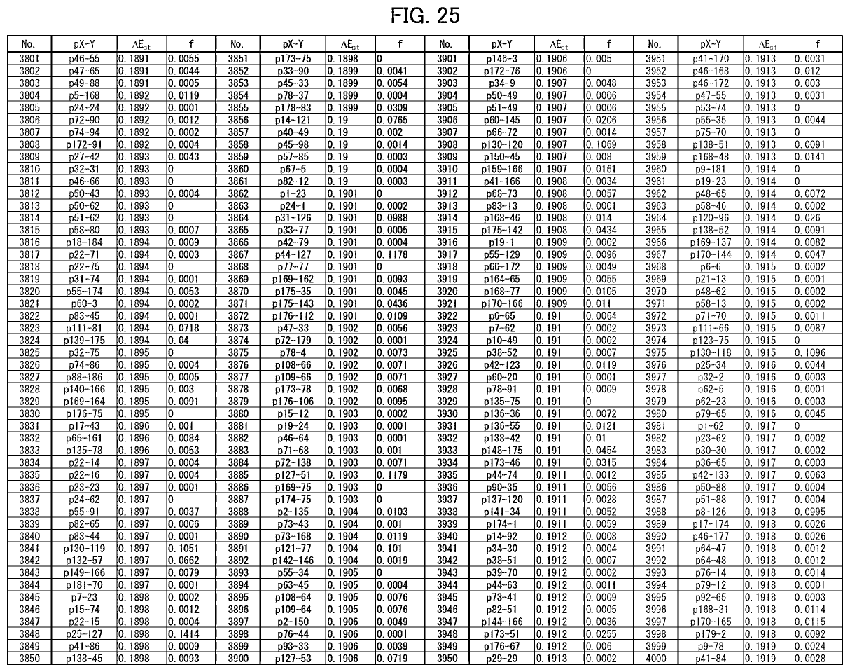

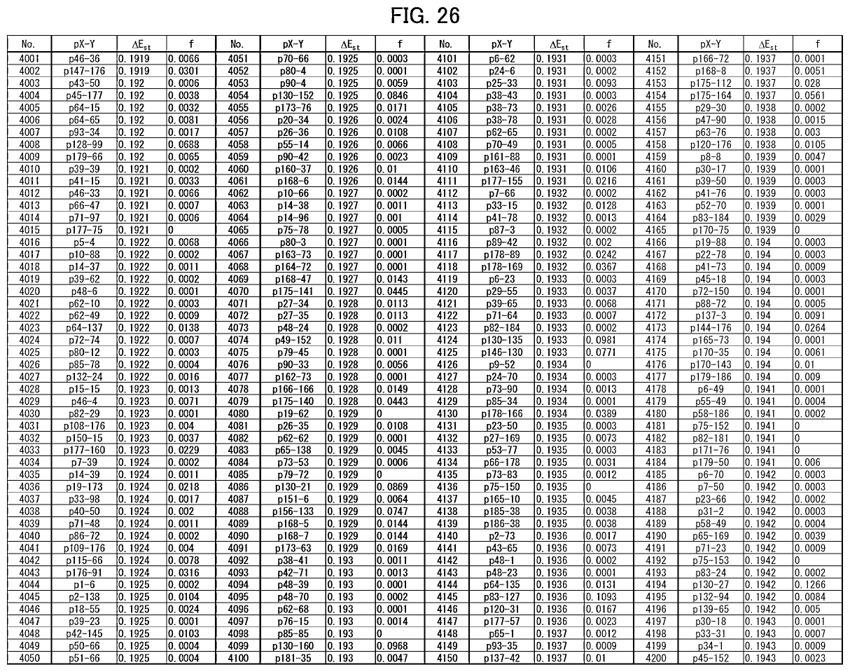

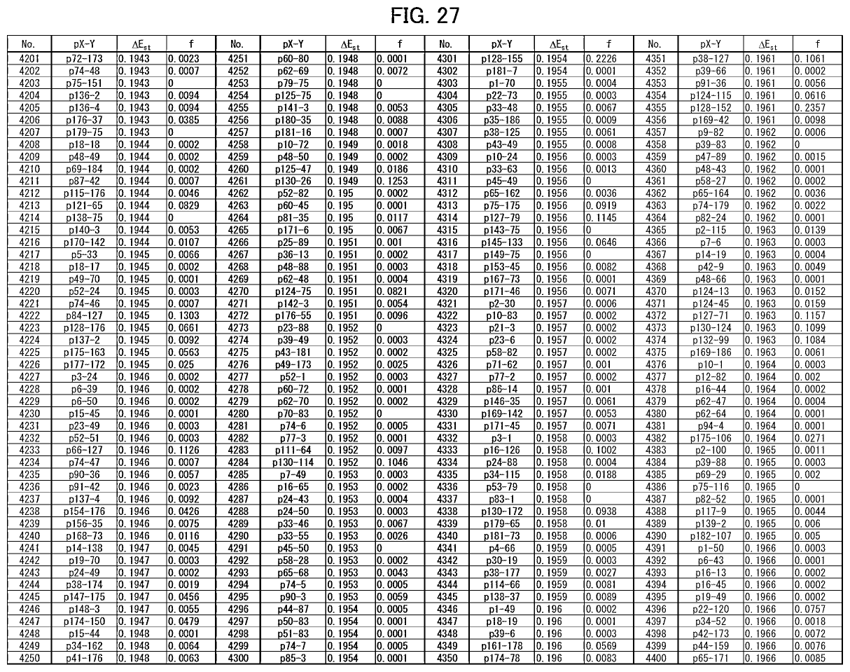

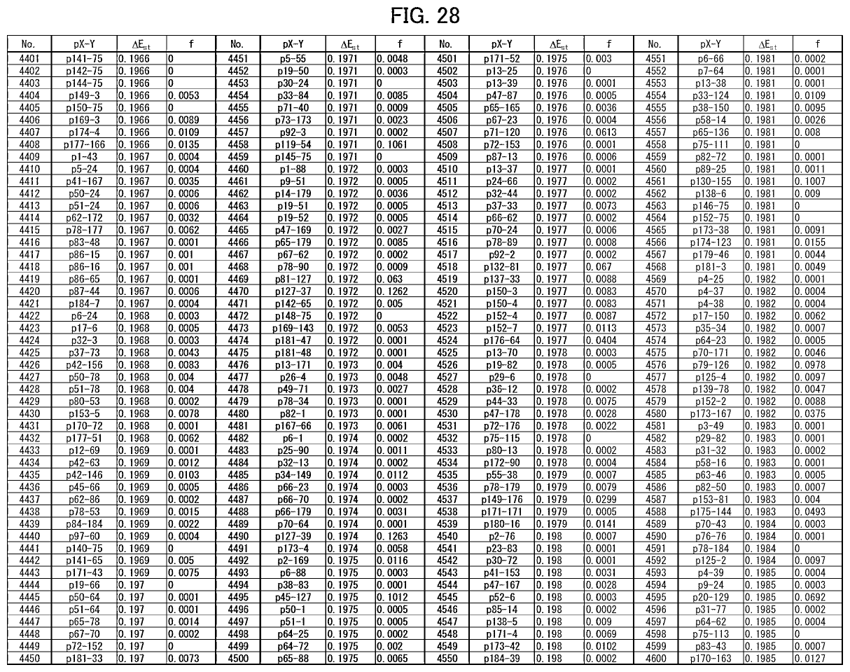

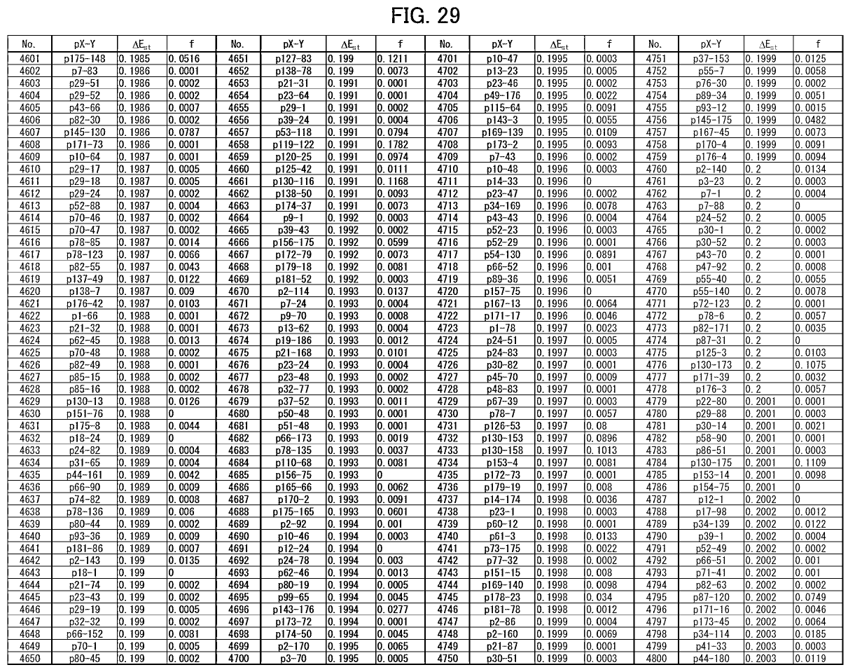

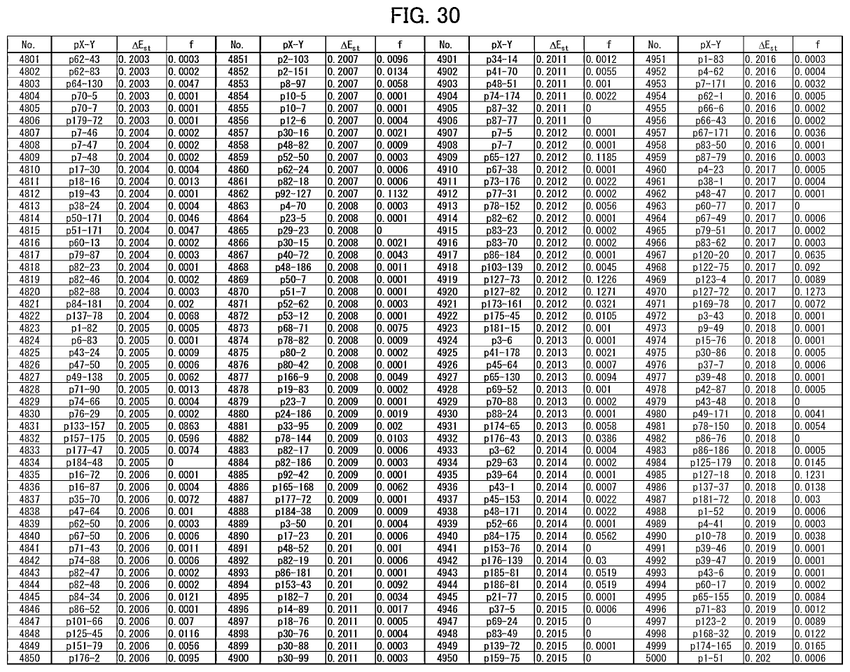

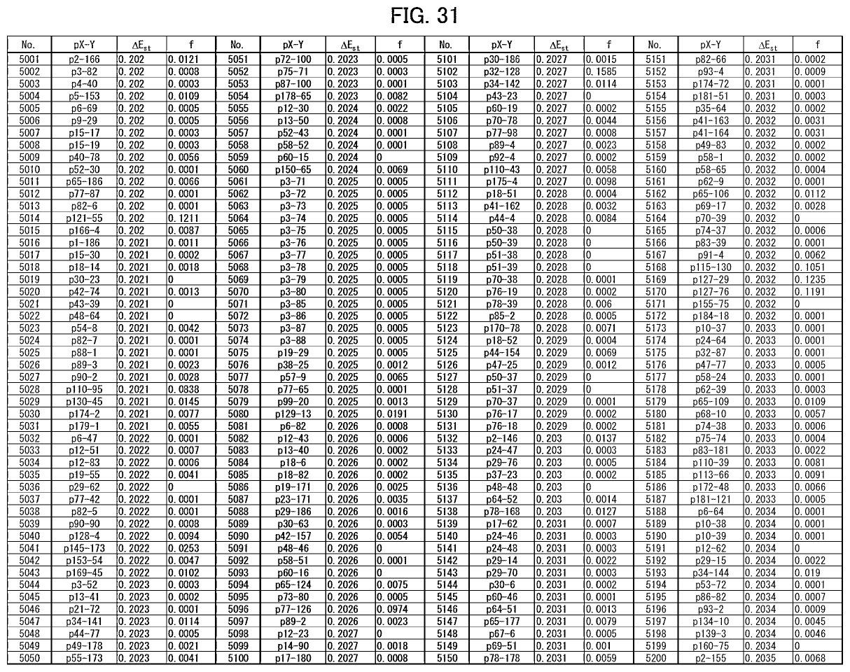

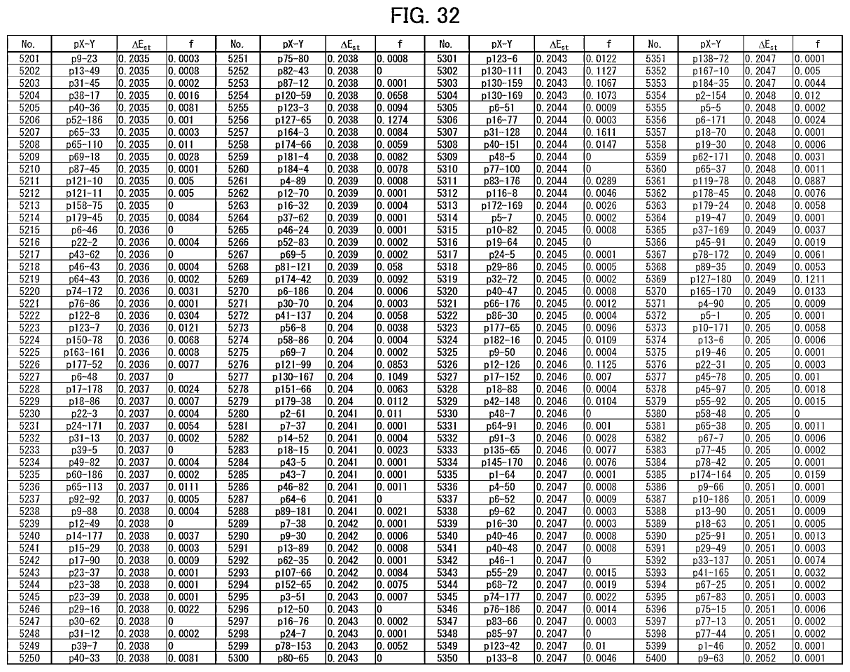

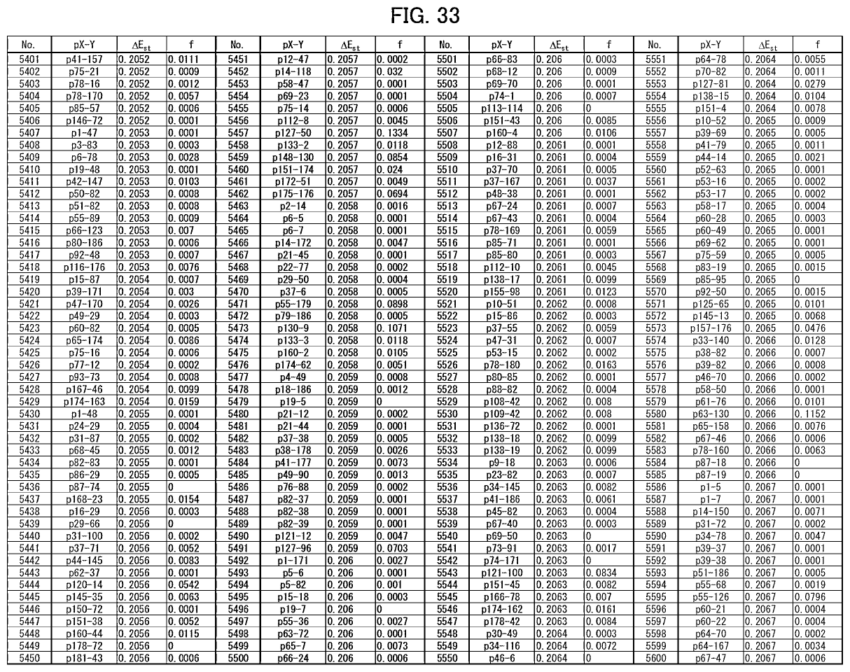

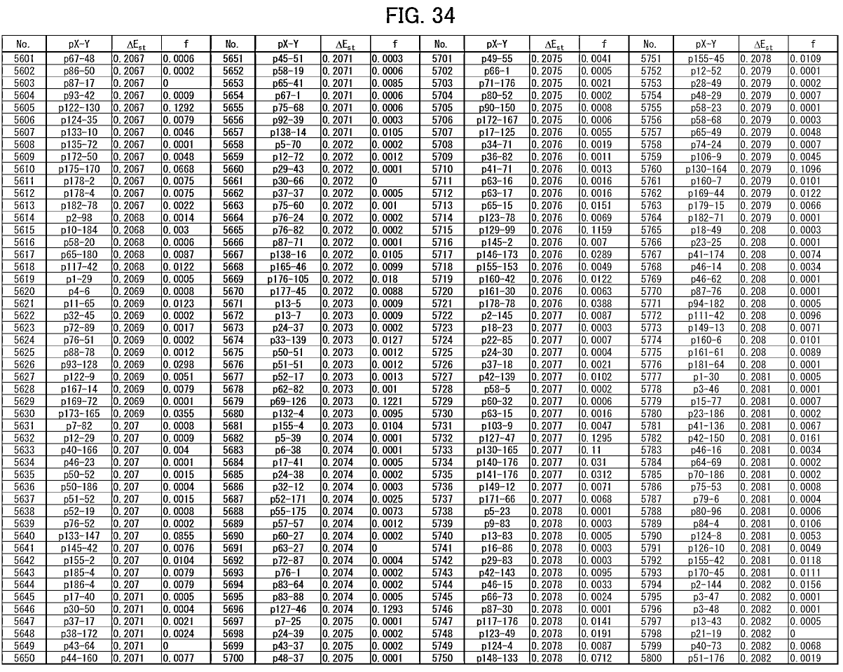

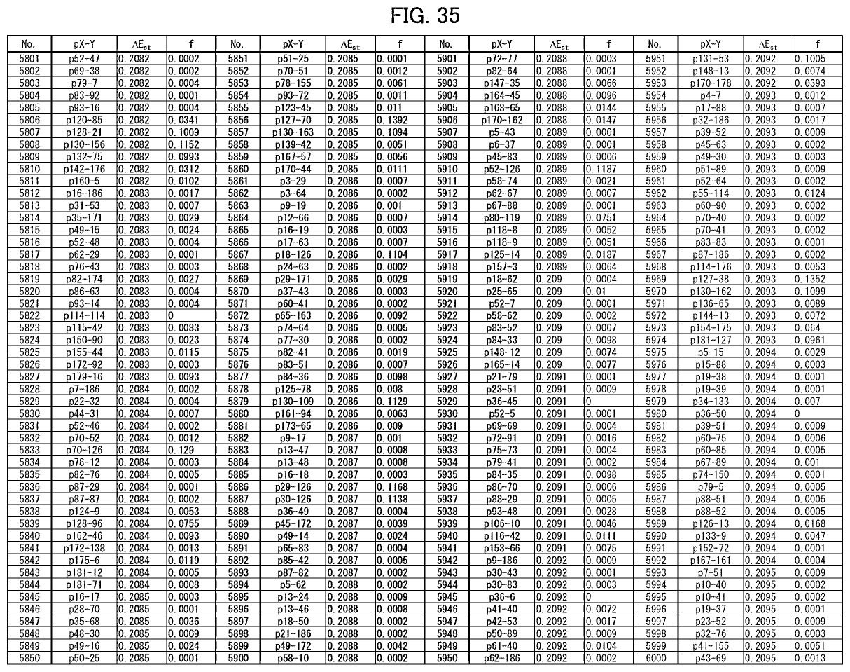

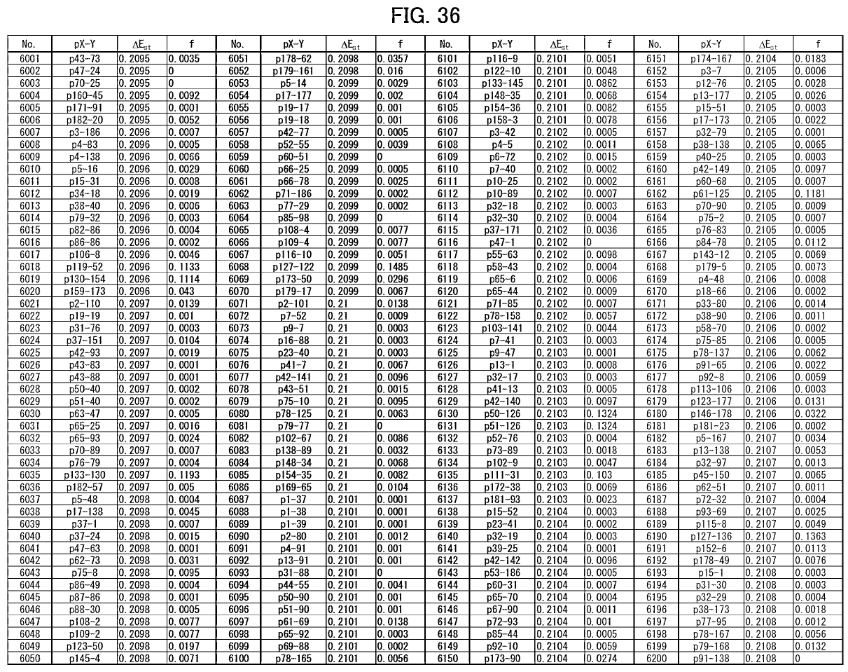

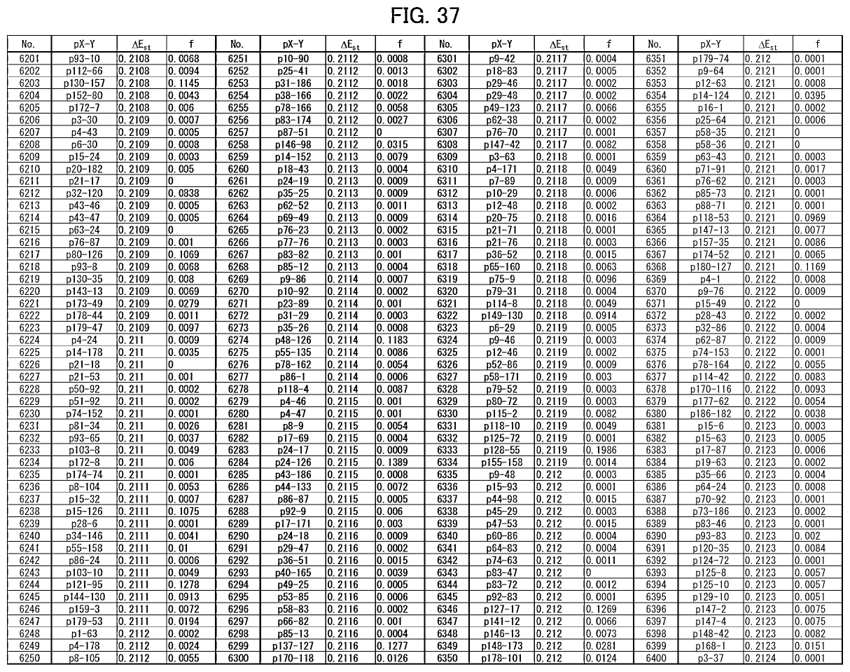

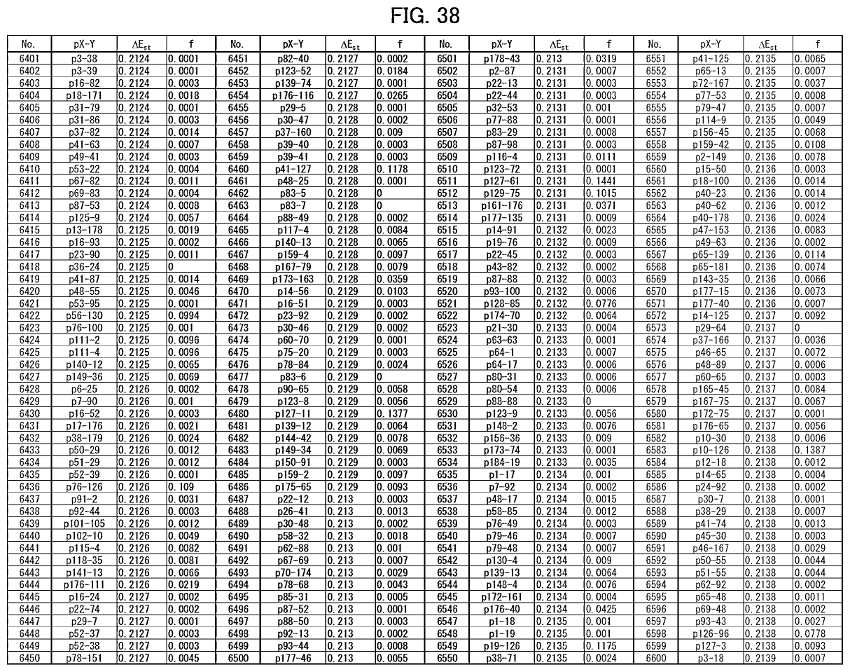

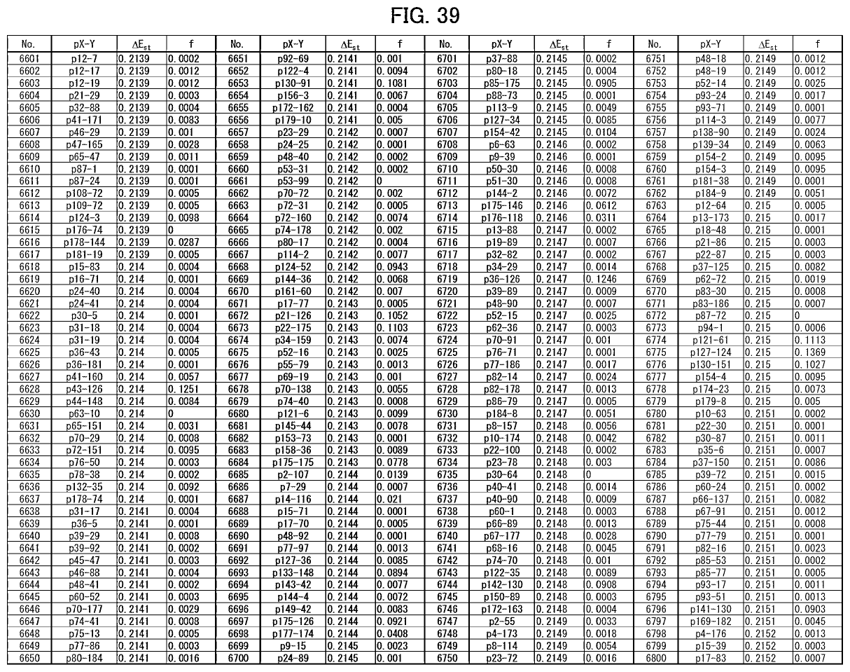

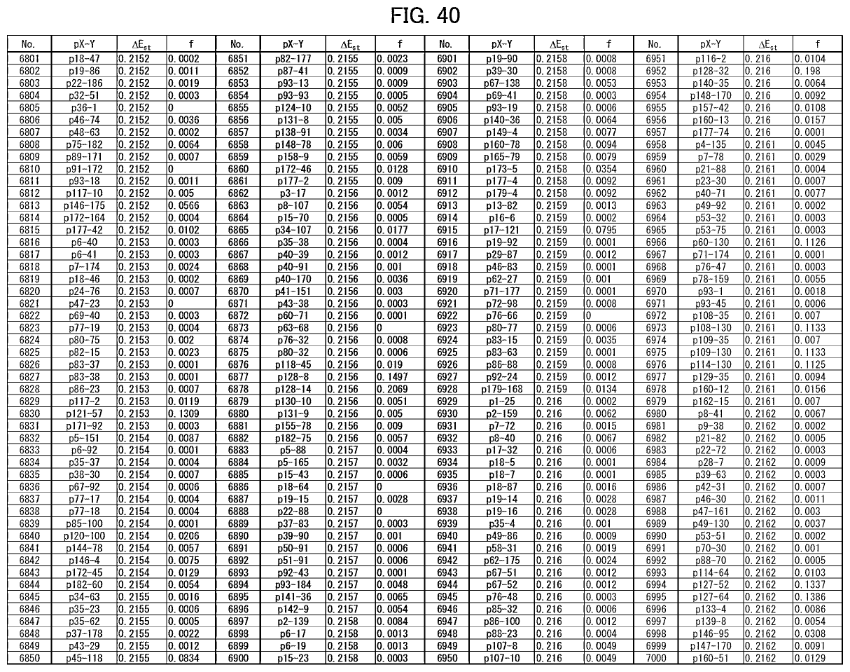

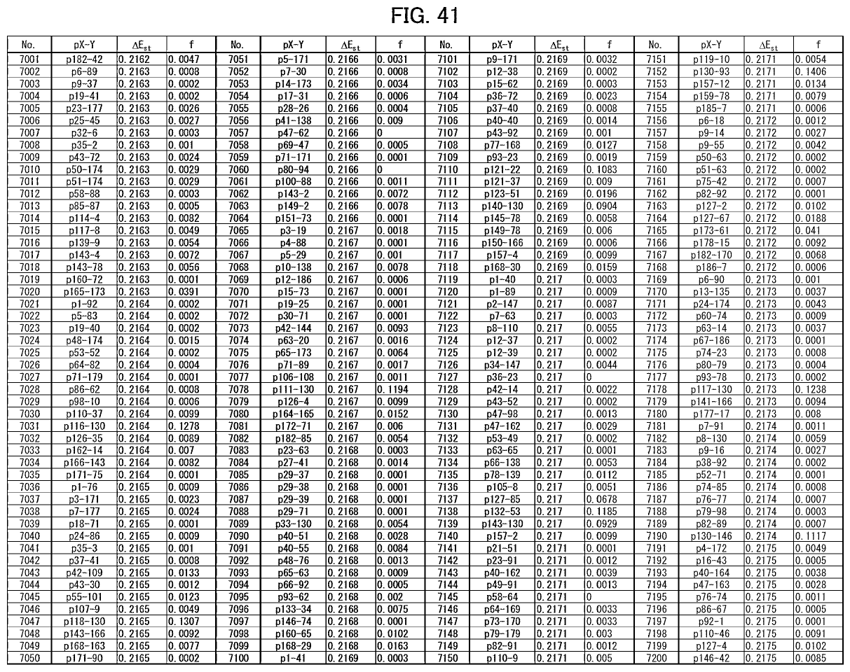

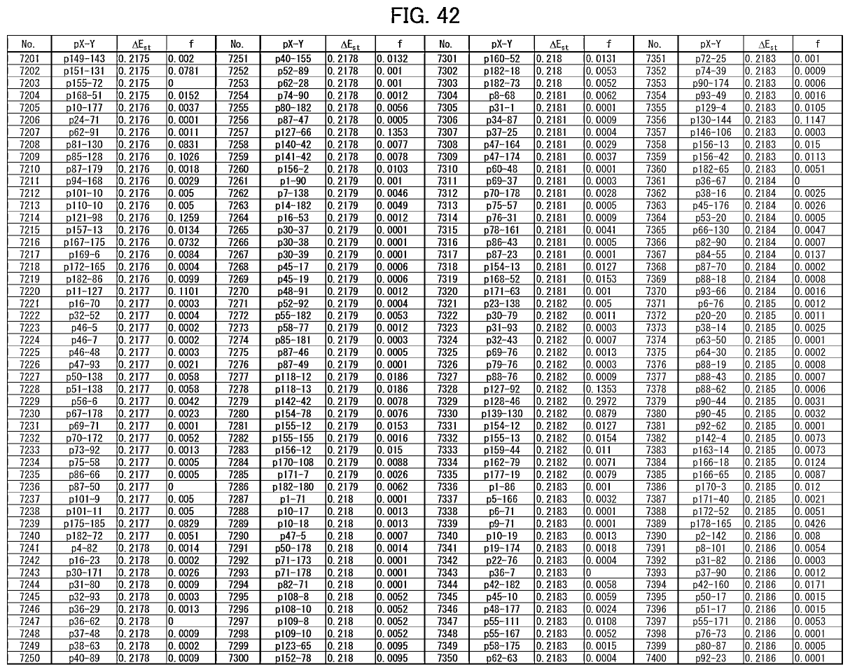

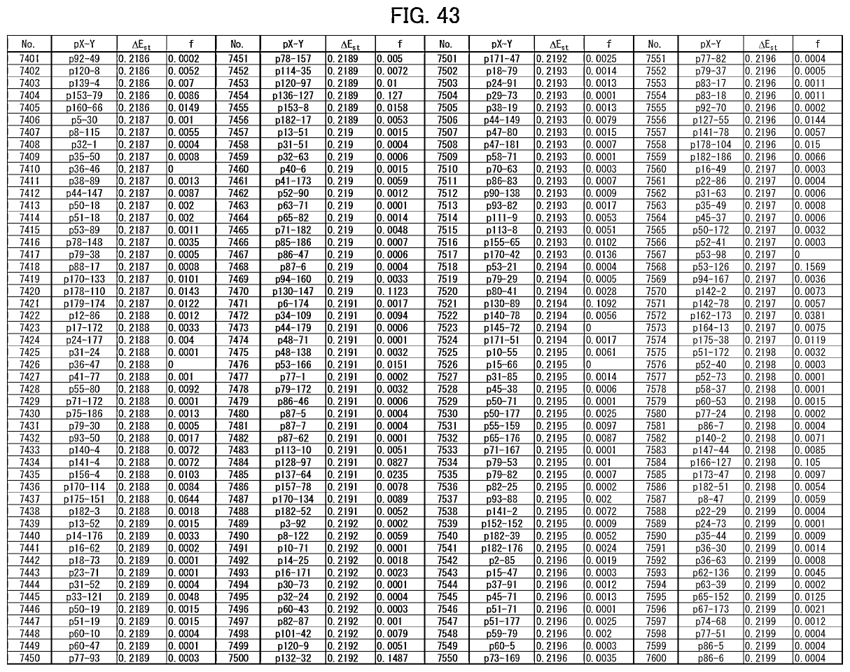

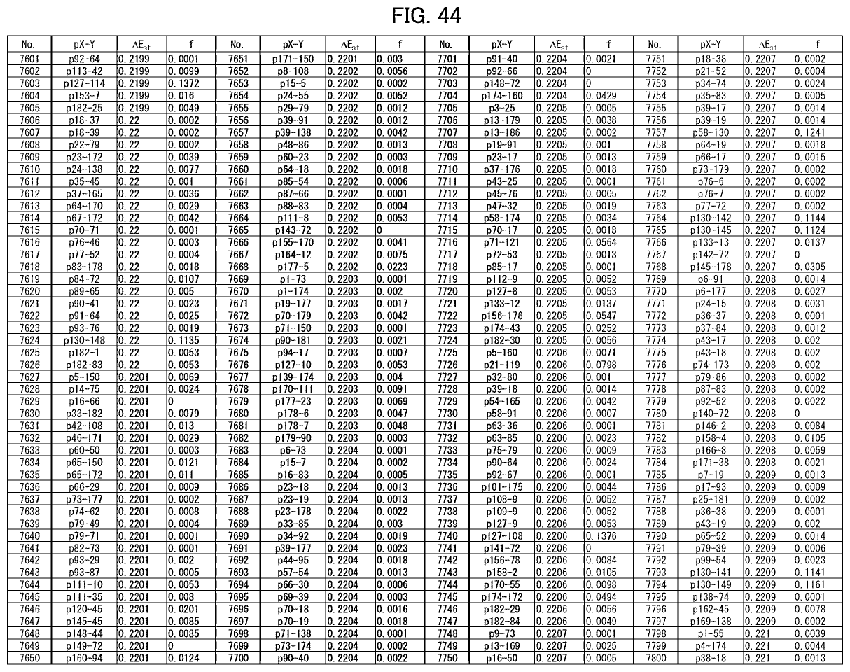

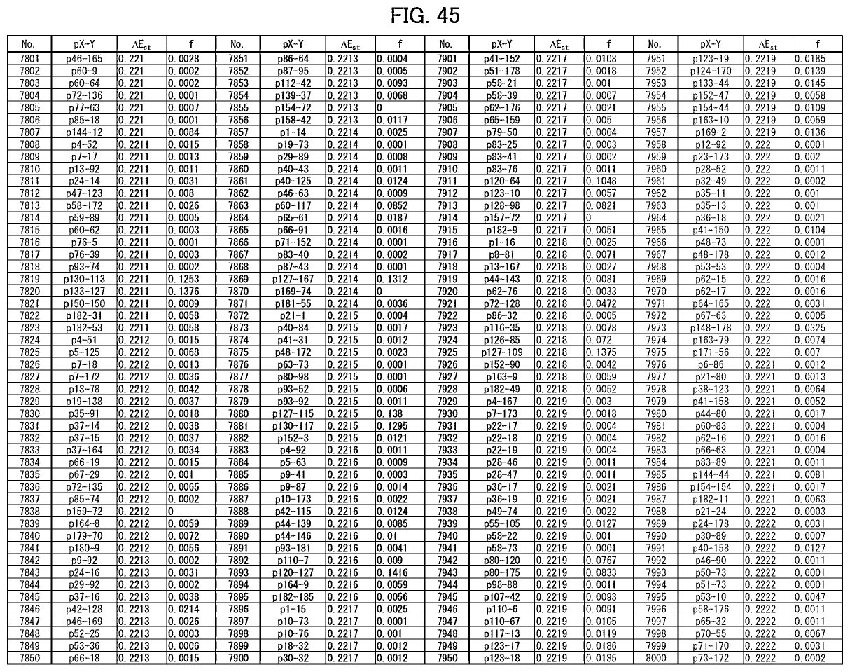

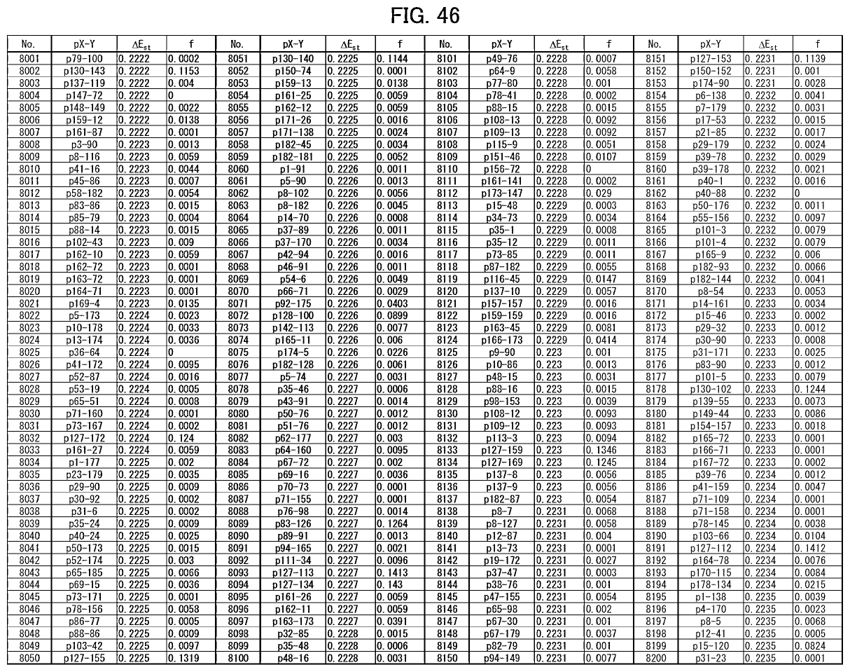

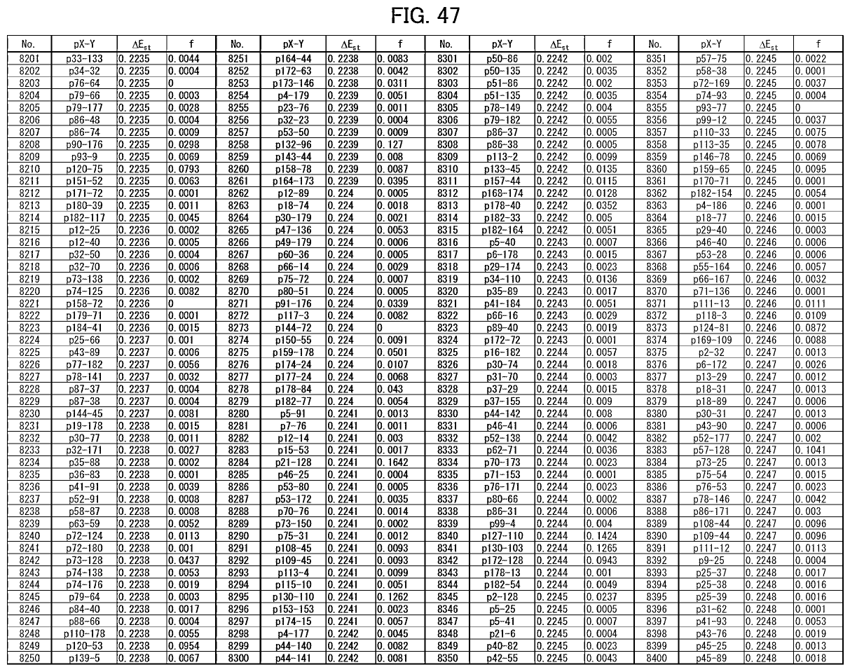

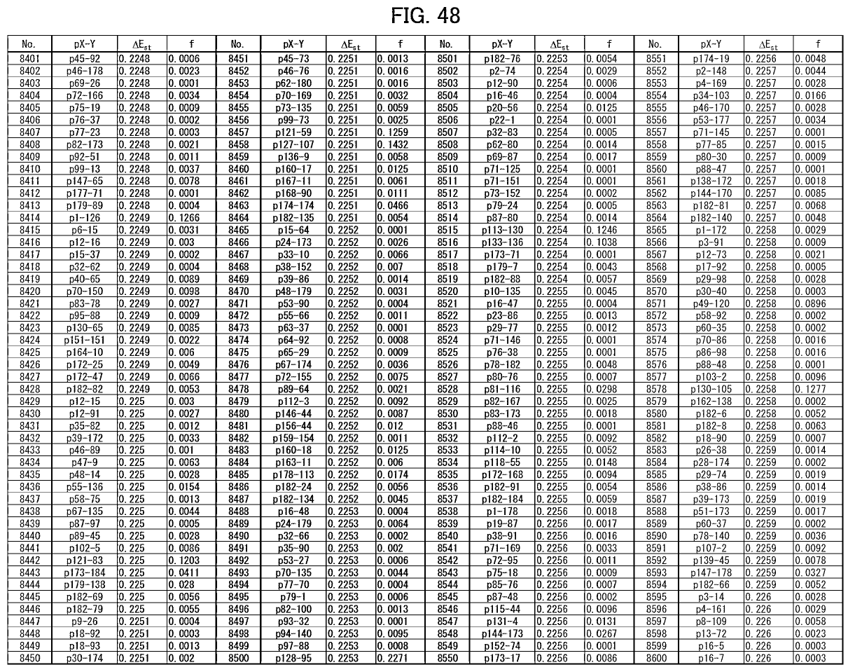

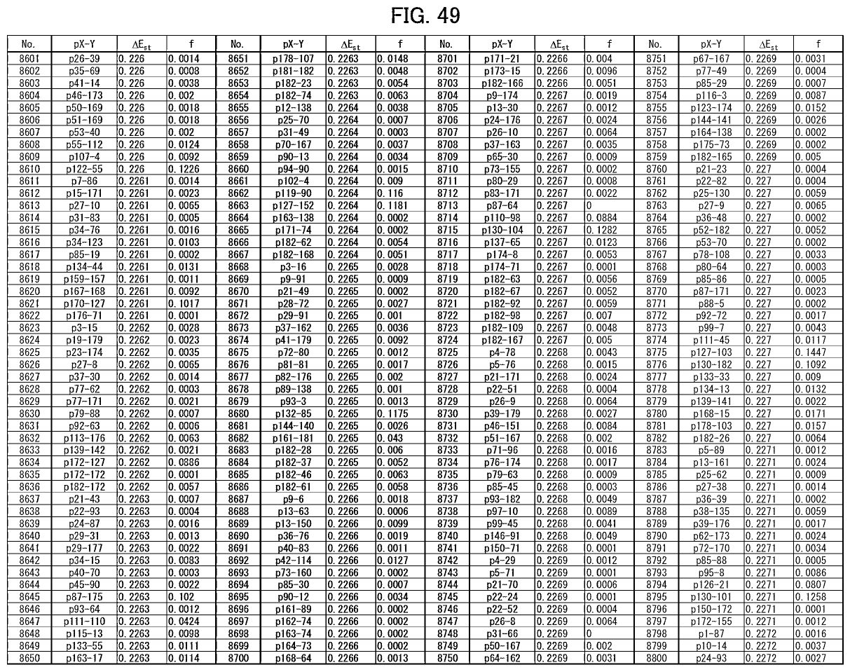

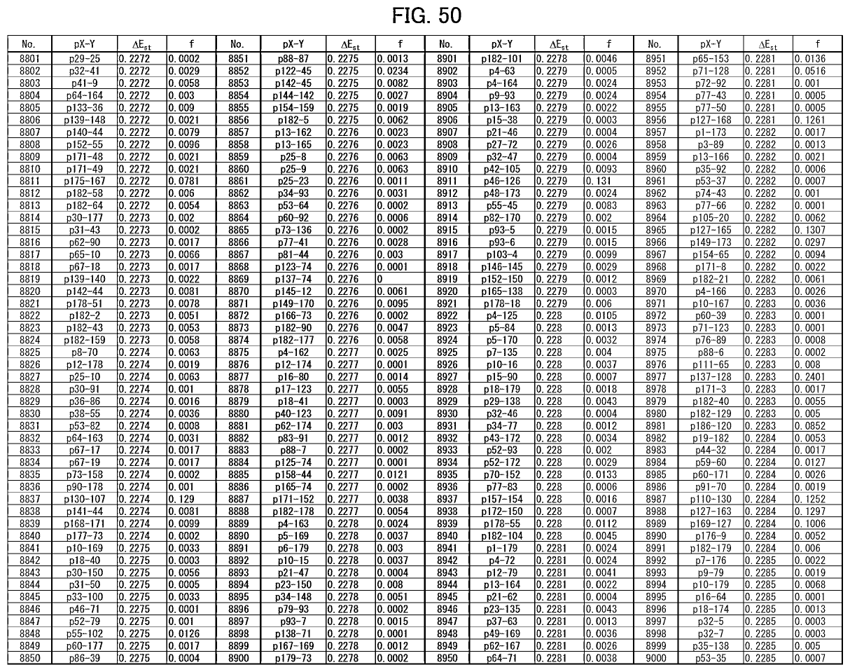

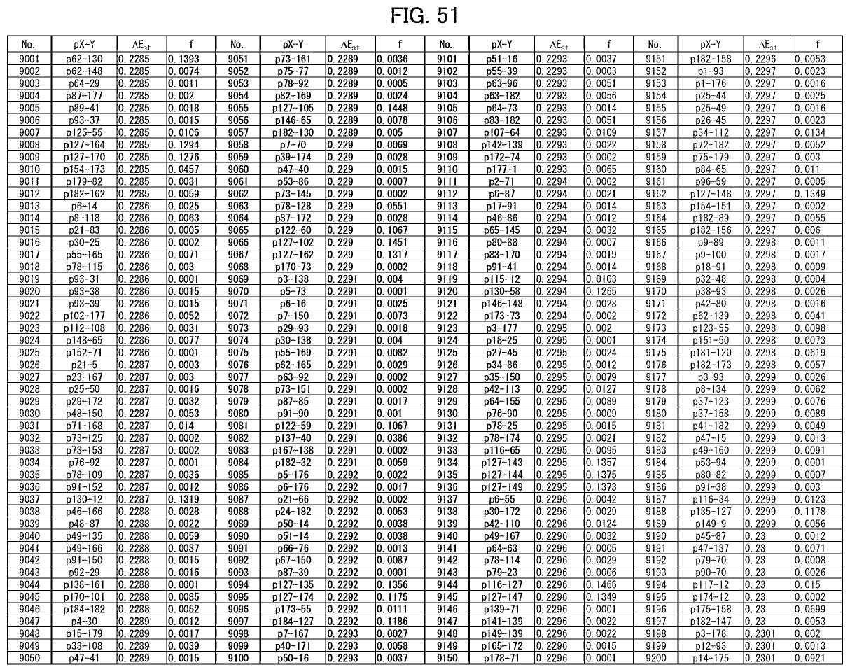

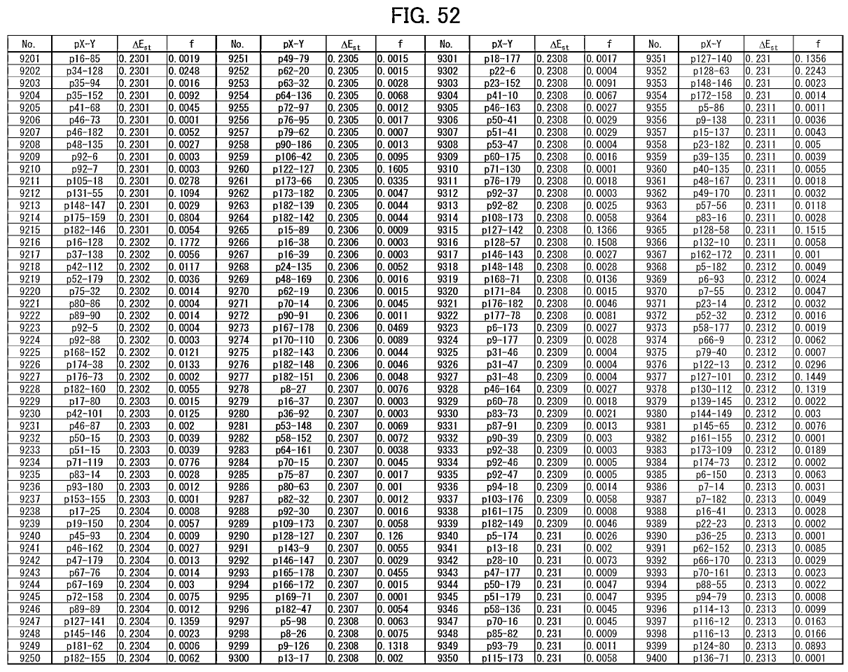

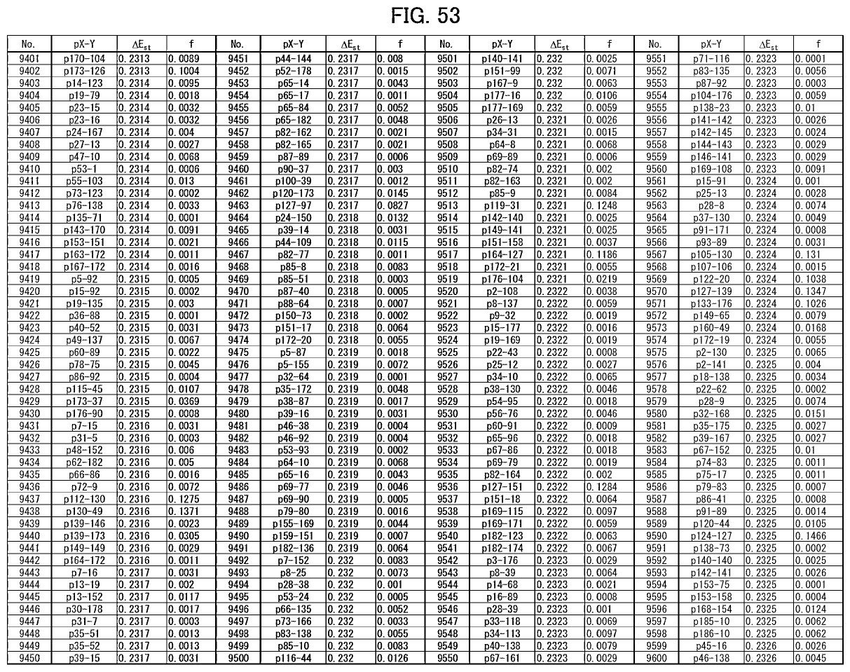

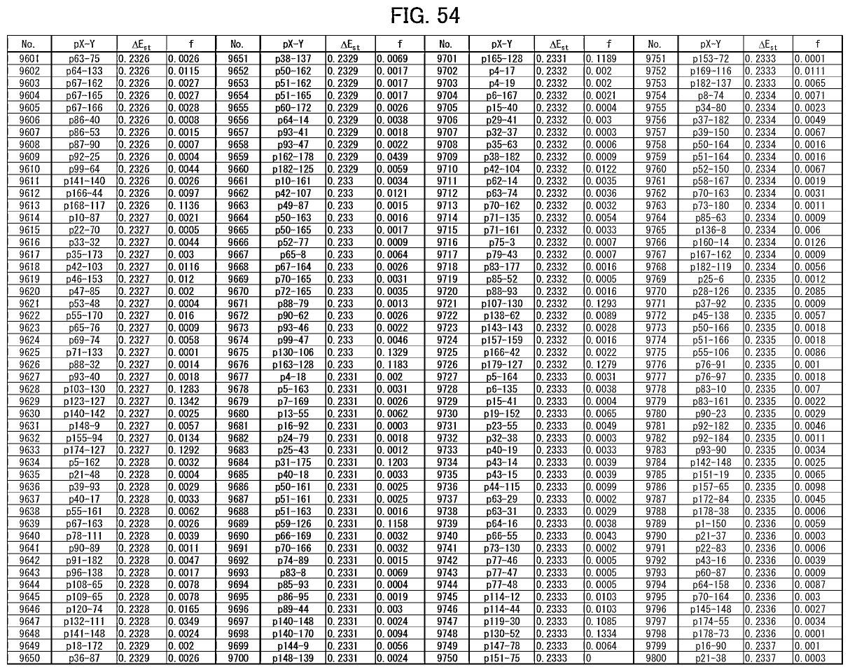

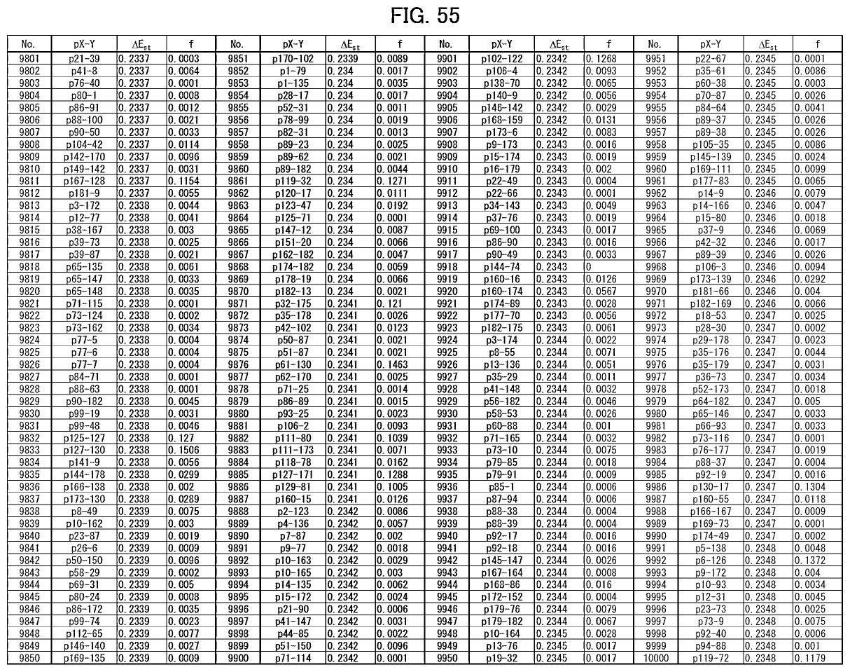

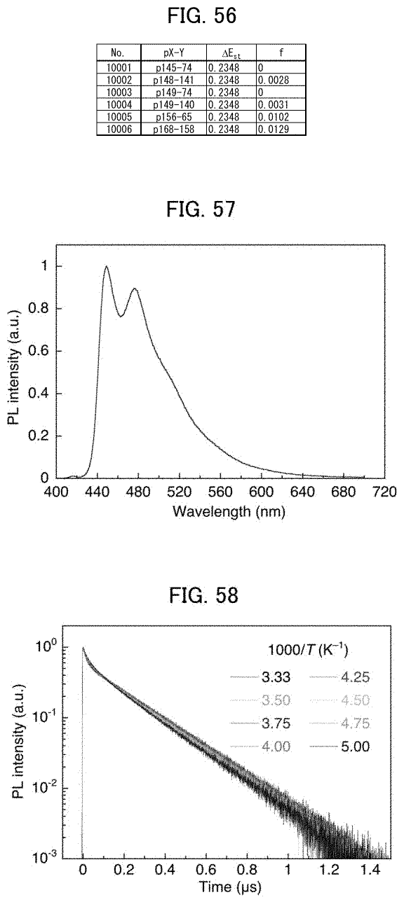

FIG. 5 indicates the results of screening calculation for the 34596 organic compounds pX-Y. FIG. 5 is a scatter diagram indicating a correlation between an energy gap ΔE_{ST} and an oscillator strength f in each of the 34596 organic compounds pX-Y. FIGS. 6 through 56 indicate 10006 organic compounds pX-Y selected in ascending order of energy gap ΔE_{ST} among the 34596 organic compounds pX-Y. FIGS. 6 through 56 are tables indicating energy gaps ΔE_{ST} and oscillator strengths f of the 10006 organic compounds pX-Y. The numbers indicated in FIGS. 6 through 56 are given in ascending order of energy gap ΔE_{ST}.

<High-Precision Calculation>

In addition, among the organic compounds pX-Y, organic compounds C, D, and E described below were subjected to structural optimization of T₁ by Unrestricted MP2 included in Gaussian 16. For the basis function, cc-pVDZ was used. Using the obtained T₁ optimized structure, an energy gap ΔE_{ST} and an oscillator strength f were calculated by EOM-CCSD or ADC(2). For the basis function, cc-pVDZ was used. Hereinafter, this calculation is referred to as “high-precision calculation”.

The organic compounds C and D exhibited relatively smaller energy gaps ΔE_{ST} and larger oscillator strengths f in the above described screening calculation. The organic compound E is an analogue of the organic compounds C and D.

The organic compound C is an organic compound p37-151 and is represented by Formula (7) below.

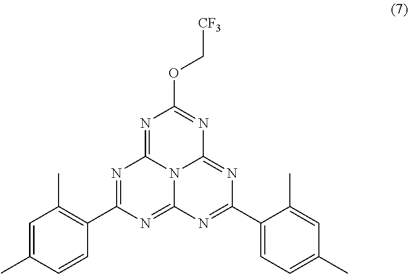

Synthesis of the organic compound C was carried out as follows. An intermediate I1 represented by Formula (8) below was dissolved in m-xylene, and aluminium chloride (1.0 g, 7.6 mmol) was added at 0° C. Stirring was carried out at 0° C. for 2 hours and at room temperature for 17 hours, and water was added. Subsequently, chloroform was added and stirred for 30 minutes, and then an organic layer was separated, dried with sodium sulfate, and concentrated. Column purification was carried out (CHCl3 100%) to obtain an organic compound C. The resulting yellow solid organic compound C was 17 mg (0.224 mmol, 7.1%).

1H NMR (600 MHz, CDCl3) δ [ppm]=2.39 (s, 6H), 2.73 (s, 6H), 4.88 (q, J=8.2 Hz, 2H), 7.11-7.13 (m, 4H), 8.19 (d, J=7.8 Hz, 2H)

MS (MALDI-TOF): 478.60 [calcd: 479.17]

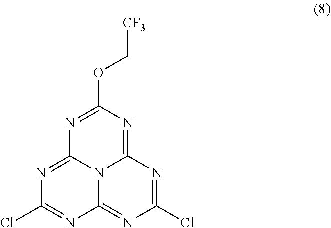 [SMILES: FC(F)(F)COC1=NC2=NC(Cl)=NC3=NC(Cl)=NC(=N1)N32]

The synthesis of the intermediate I1 was carried out as follows. 2,2,2-trifluoroethanol (199 mL, 2.78 mmol) was dissolved in tetrahydrofuran (10 mL) and sodium hydride (121 mg, 3.0 mmol) was added at 0° C. After stirring for 30 minutes, the resulting mixture was slowly dripped into a tetrahydrofuran solution (20 mL) of cyameluric acid (700 mg, 2.53 mmol) at 0° C., stirred at 0° C. for 2 hours, and further stirred at room temperature for 1 hour. The reaction liquid was concentrated under reduced pressure, and thus the intermediate I1 was obtained.

The organic compound D is an organic compound p37-107 and is represented by Formula (9) below.

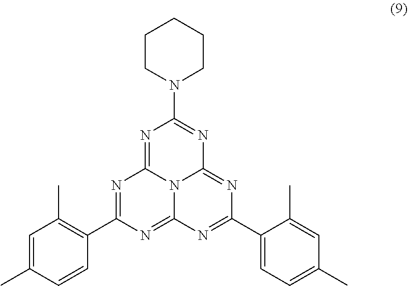

Synthesis of the organic compound D was carried out as follows. An intermediate I2 represented by Formula (10) below was dissolved in m-xylene (20 mL), aluminum chloride (980 mg, 0.79 mmol) was added at room temperature under an argon atmosphere, and the resulting mixture was stirred for 17 hours. Water was added and the reaction was stopped. After extraction with chloroform, an organic layer was dried with sodium sulfate and concentrated. Purification was carried out with a column (AcOEt:CHC₁₃=0:100-5:95) to obtain an object. The resulting yellow solid organic compound D was 25 mg (0.054 mmol, 2.2%).

¹H NMR (600 MHz, CDCl₃) δ [ppm]=1.66-1.71 (m, 6H), 2.36 (s, 6H), 2.67 (s, 6H), 3.96 (br s, 4H), 7.06-7.07 (m, 4H), 7.99 (d, J=8.4 Hz, 2H)

MS (MALDI-TOF): 465.71 [calcd: 464.24]

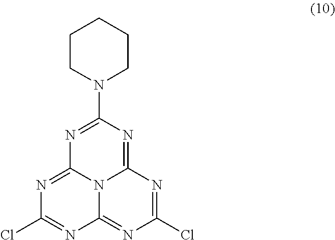 [SMILES: ClC1=NC2=NC(Cl)=NC3=NC(N4CCCCC4)=NC(=N1)N23]

The synthesis of the intermediate 12 was carried out as follows. Cyameluric acid chloride (677 mg, 2.45 mmol) was dissolved in tetrahydrofuran (20 mL) and piperidine (266 mL, 2.7 mmol) was added at room temperature. 30 minutes later, the temperature was raised to 50° C., and stirring was carried out for 45 minutes. After the temperature was returned to room temperature, the reaction liquid was concentrated under reduced pressure, and thus the intermediate 12 was obtained.

The organic compound E is an organic compound p37-37 and is represented by Formula (11) below.

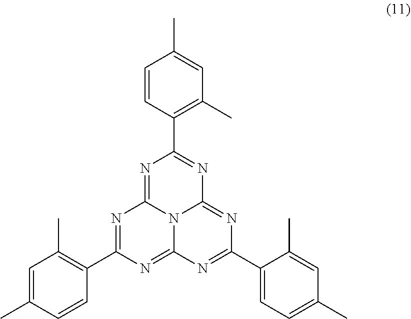

Synthesis of the organic compound E was carried out as follows. Cyameluric acid (623 mg, 2.26 mmol) was dissolved in m-xylene (20 mL) and diphenylamine (420 mg, 2.49 mmol) was added at room temperature. After stirring was carried out for 2.5 hours, the temperature was raised to 50° C., and stirring was carried out for another 2 hours. The resulting mixture was cooled to 0° C., aluminium chloride (904 mg, 6.8 mmol) was added, the resulting mixture was stirred at room temperature for 17 hours, and then water was added. Subsequently, 30 minutes later, chloroform was added, an organic layer was separated, and dried with sodium sulfate. Then, concentration and column purification were carried out (CHCl₃ 100%) to obtain an object. The resulting white solid organic compound p37-37 was 70 mg (0.14 mmol, 6.4%).

¹H NMR (600 MHz, CDCl₃) δ [ppm]=2.34 (s, 6H), 2.63 (s, 6H), 7.03 (br s, 4H), 7.28-7.31 (m, 6H), 7.38 (t, J=7.8 Hz, 4H), 7.97 (d, J=8.4 Hz, 2H)

MS (MALDI-TOF): 549.94 [calcd: 548.24]

Table 2 indicates the results of high-precision calculation for the organic compounds C, D, and E.

TABLE 2

ΔE_{ST} (meV)

| | EOM-CCSD/cc-pVDZ | ADC(2)/cc-pVDZ |
|---|---|---|
| Organic | −12 | −34 |
| Compound C | | |
| Organic | 10 | −118 |
| Compound D | | |
| Organic | 10 | −86 |
| Compound E | | |

<Evaluation of Luminescence Property>

Emission spectra of a toluene solution of the organic compound C (concentration of 8.0×10⁻⁵ M) and a mixed thin film containing the organic compound C and 2,8-bis(diphenylphosphoryl)dibenzo[b,d]furan (PPF) (concentration of 10 wt %) prepared by vacuum deposition were measured using a Fluoromax-4 spectrophotofluorometer manufactured by HORIBA.

Luminescent quantum yields of the toluene solution of the organic compound C and the mixed thin film were measured using a C9920 integrating sphere manufactured by Hamamatsu Photonics K.K. Luminescence lifetimes τ of the toluene solution of the organic compound C and the mixed thin film were measured using a Fluorolog-3 fluorescence lifetime measuring device manufactured by HORIBA. The luminescence lifetime τ is also referred to as a delayed fluorescence lifetime. The above measurements were carried out under an inert nitrogen atmosphere with an excitation light wavelength of 370 nm. The measurement of the delayed fluorescence lifetime τ was carried out while changing the temperature using a cryostat CoolSpeK manufactured by UNISOKU. The temperature dependence of the obtained delayed fluorescence lifetime τ was analyzed by a mathematical formula (3) assuming a thermal equilibrium between S₁ and T₁, and experimental values of the energy gap ΔE_{ST} and the radiative decay rate constant kᵣ were estimated.

$${τ}^{- 1} = {k}_{DF} = \frac{1}{3} {k}_{r} exp ( \frac{- Δ {E}_{ST}}{{k}_{B} T} ) \qquad ( 3 )$$

As with the organic compound C, toluene solutions of organic compounds D, E, F, G, H, I, J, K, L, and M were adjusted to evaluate luminescence properties.

The organic compound F is an organic compound p1-151 and is represented by Formula (12) below.

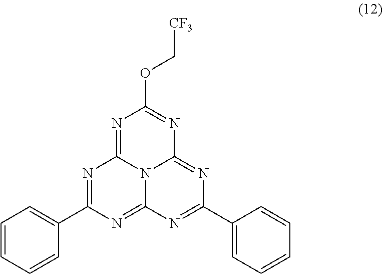 [SMILES: FC(F)(F)COC1=NC2=NC(c3ccccc3)=NC3=NC(c4ccccc4)=NC(=N1)N23]

Synthesis of the organic compound F was carried out as follows. The intermediate I1 was dissolved in benzene (15 mL) and aluminium chloride (884 mg, 6.6 mmol) was added at 0° C. and stirred for 5 minutes. Further, stirring was carried out at room temperature for 10 minutes and at 70° C. for 19 hours. Water was added and stirred for 30 minutes, then chloroform was added, and an organic layer was separated. The organic layer was dried with sodium sulfate. Then, concentration and column purification were carried out (CHCl₃ 100%) to obtain an object. The resulting yellow solid organic compound F was 15 mg (0.035 mmol, 1.6%).

¹H NMR (600 MHz, CDCl₃) δ [ppm]=4.92 (q, J=8 Hz, 2H), 7.53 (dd, J=7.8 Hz, 4H), 7.68 (dd, J=7.2 Hz, 2H), 8.56 (d, J=7.2 Hz, 4H)

MS (MALDI-TOF): 469.61 [calcd: 468.20]

The organic compound G is an organic compound p7-151 and is represented by Formula (13) below.

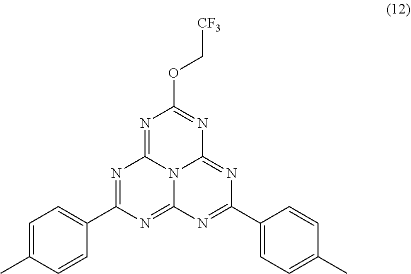

Synthesis of the organic compound G was carried out as follows. The intermediate I1 was dissolved in toluene (10 mL), aluminum chloride (872 mg, 6.5 mmol) was added at 0° C., and the resulting mixture was stirred at 0° C. for 30 minutes and at room temperature for 19 hours. Water was added and subsequently chloroform was added, the resulting mixture was stirred for 30 minutes, and then an organic layer was separated. The organic layer was dried with sodium sulfate. Then, concentration and column purification were carried out (CHCl₃ 100%) to obtain an object. The resulting yellow solid organic compound G was 90 mg (0.20 mmol, 9.2%).

¹H NMR (600 MHz, CDCl₃) δ [ppm]=2.46 (s, 6H), 4.90 (q, J=8 Hz, 2H), 7.33 (d, J=7.8 Hz, 4H), 8.45 (d, J=8.4 Hz, 4H)

MS (MALDI-TOF): 452.64 [calcd: 451.14]

The organic compound H is an organic compound p7-107 and is represented by Formula (14) below.

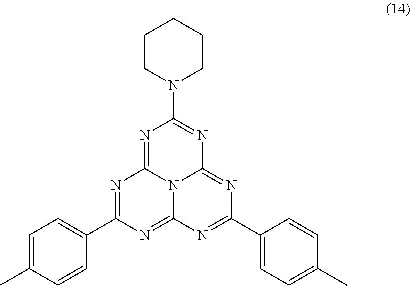

Synthesis of the organic compound H was carried out as follows. Cyameluric acid chloride (100 mg, 0.36 mmol) was dissolved in toluene (3 mL), and piperidine (36 mL, 0.36 mmol) was added at room temperature. 5 minutes later, the temperature was raised to 100° C., stirring was carried out for 30 minutes, and then the temperature was returned to room temperature. Aluminum chloride (106 mg, 0.79 mmol) was added, the resulting mixture was stirred at 100° C. for 1 hour, then the temperature was returned to room temperature, and water was added. An organic layer was separated and dried with sodium sulfate. Then, concentration and column purification were carried out (AcOEt:CHCl₃=0:100-1:20) to obtain an object. The resulting yellow solid organic compound H was 19 mg (0.044 mmol, 12.1%).

¹H NMR (600 MHz, CDCl₃) δ [ppm]=1.68-1.72 (m, 6H), 2.44 (s, 6H), 3.99 (br s, 4H), 7.29 (d, J=7.8 Hz, 4H), 8.44 (d, J=7.8 Hz, 4H)

The organic compound I is an organic compound p64-166 and is represented by Formula (15) below.

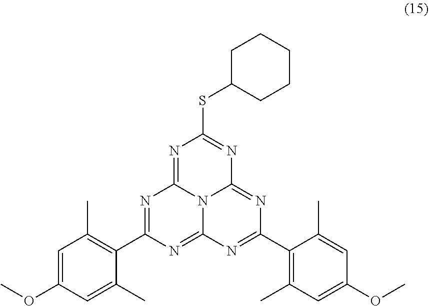

Synthesis of the organic compound I was carried out as follows. To a dichloromethane (11.8 mL) solution of an intermediate I3 (608 μL, 4.3 mmol) represented by Formula (16) below, aluminum chloride (616 mg, 4.6 mmol) was added at room temperature and stirred for 40 minutes. A dichloromethane (12 mL) solution of a compound 2 was slowly added and stirred at room temperature for 20.5 hours. A 1-M sodium hydroxide aqueous solution (16 mL) was added at 0° C., stirred at room temperature for 4 hours, and then filtered using cerite. A 20% sodium chloride aqueous solution was added to the reaction liquid, and then an organic layer was separated and dried with anhydrous sodium sulfate. Then concentration and column purification (CH₂Cl₂—CH₂Cl₁:MeOH=9:1) were carried out to obtain a crude body (117 mg). The crude body was purified (SunFire, Hexane/EtOAc=82:18) in a preparative column to obtain an organic compound I as a first peak. The resulting yellow solid organic compound I was 8.4 mg (0.015 mmol, 1.4%).

¹H NMR (600 MHz, CDCl₃) δ [ppm]=0.80-0.92 (br, 2H), 1.20-1.38 (br, 2H), 1.38-1.50 (br, 2H), 1.69-1.79 (br, 2H), 2.01-2.11 (br, 2H), 2.41 (s, 12H), 3.80 (s, 6H), 3.87-3.96 (br, 1H), 6.60 (s, 4H)

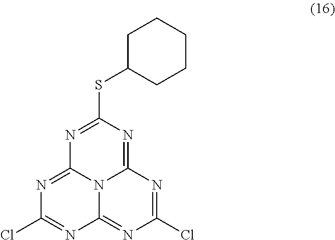 [SMILES: ClC1=NC2=NC(Cl)=NC3=NC(SC4CCCCC4)=NC(=N1)N23]

The synthesis of the intermediate 13 was carried out as follows. Diisopropyl ethylamine (93 μL, 0.54 mmol) and cyclohexanethiol (66 μl, 0.54 mmol) were added to a toluene (8.9 mL) suspension of a mixture (536 mg, 1.1 mmol) of cyameluric acid chloride-potassium trichloride at room temperature, and then heating and reflux were carried out for 14 hours. The temperature was cooled to room temperature, an insoluble matter was filtered, and then concentration was carried out under reduced pressure to obtain the intermediate 13.

The organic compound J is an organic compound p107-4 which is represented by Formula (17) below.

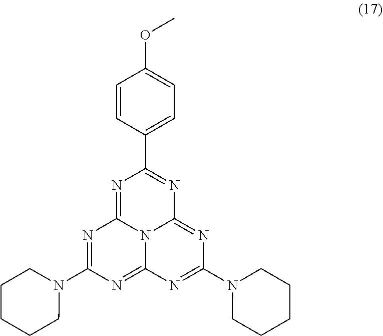 [SMILES: COc1ccc(C2=NC3=NC(N4CCCCC4)=NC4=NC(N5CCCCC5)=NC(=N2)N34)cc1]

Synthesis of the organic compound J was carried out as follows. Cyameluric acid chloride (70 mg, 0.25 mmol) was added to a dichloromethane (3 mL) solution of aluminium chloride (133 mg, 1.0 mmol) and methoxybenzene (41 μL, 0.38 mmol) at 0° C. 10 minutes later, the reaction solution was heated to room temperature and stirred for 17 hours. An excessive amount of piperidine (0.5 mL) was added and stirred for 30 minutes, and then diluted with water and chloroform. A separated organic layer was concentrated and then purified with a column (CH₂Cl₂ 100%-AcOEt:CH₂Cl₂=1:4) to obtain an object. The resulting yellow solid organic compound J was 6.8 mg (0.015 mmol, 6.1%).

¹H NMR (600 MHz, CDCl₃) δ [ppm]=1.64-1.69 (m, 12H), 3.91 (t, 4H), 3.95 (t, 4H), 6.93 (d, J=9 Hz, 2H), 8.48 (d, J=8.4 Hz, 2H)

¹³C NMR (600 MHz, CDCl₃) δ [ppm]=24.44, 26.16, 45.50, 55.44, 113.47, 127.71, 132.10, 155.21, 156.09, 161.40, 163.88, 172.87

MS (FD-TOF): 445.2342 [M]⁺, calcd. for C₂₃H₂₇N₉O (445.2339)

The organic compound K is an organic compound p107-107, which is represented by Formula (18) below.

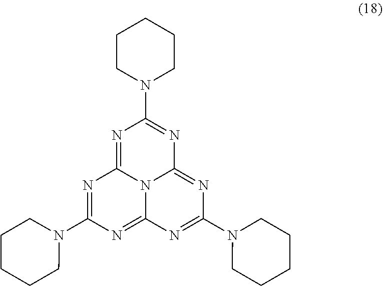 [SMILES: C1CCN(C2=NC3=NC(N4CCCCC4)=NC4=NC(N5CCCCC5)=NC(=N2)N34)CC1]

Synthesis of the organic compound K was carried out as follows. Cyameluric acid chloride (70 mg, 0.25 mmol) was added to a dichloromethane (3 mL) solution of aluminium chloride (133 mg, 1.0 mmol) and methoxybenzene (41 μL, 0.38 mmol) at 0° C. 10 minutes later, the reaction solution was heated to room temperature and stirred for 17 hours. An excessive amount of piperidine (0.5 mL) was added and stirred for 30 minutes, and then diluted with water and chloroform. A separated organic layer was concentrated and then purified with a column (CH₂Cl₂ 100%-AcOEt:CH₂Cl₂=1:4) to obtain an object. The resulting white solid organic compound K was 30 mg (0.071 mmol, 28.4%).

¹H NMR (600 MHz, CDCl₃) δ [ppm]=1.59-1.65 (m, 18H), 3.87 (t, 12H)

¹³C NMR (600 MHz, CDCl₃) δ [ppm]=24.50, 26.12, 45.15, 155.11, 161.59

MS (FD-TOF): 422.2658 [M]⁺, calcd. for C₂₁H₃₀N₁₀ (422.2655)

The organic compound L is an organic compound p105-105, which is represented by Formula (19) below.

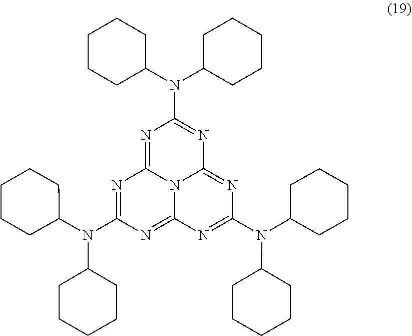 [SMILES: C1CCC(N(C2=NC3=NC(N(C4CCCCC4)C4CCCCC4)=NC4=NC(N(C5CCCCC5)C5CCCCC5)=NC(=N2)N34)C2CCCCC2)CC1]

Synthesis of the organic compound L was carried out as follows. Dicyclohexylamine (1.39 ml, 7.0 mmol) was added to a toluene (5.5 mL) suspension of a mixture (501 mg, 1.0 mmol) of cyameluric acid chloride-potassium trichloride at room temperature. Stirring was carried out at 100° C. for 21 hours using a heat block. Dichloromethane and water were added to the reaction liquid, then an organic layer was separated, dried with anhydrous sodium sulfate, and concentrated under reduced pressure to obtain a crude body (1.088 g). Recrystallization from dichloromethane of the crude body and column purification (CH₂C12) were carried out to obtain an organic compound L. The resulting white solid organic compound L was 220.7 mg (0.310 mmol, 31.0%).

¹H NMR (600 MHz, CDCl₃) δ [ppm]=1.10-1.22 (br, 12H), 1.27-1.44 (br, 18H), 1.58-1.74 (br, 18H), 1.80 (d, J=12.0 Hz, 12H), 2.08-2.94 (br, 6H)

MS (MALDI-TOF): 712.251 [M+H=712.067]

The organic compound M is an organic compound p144-144, which is represented by Formula (20) below.

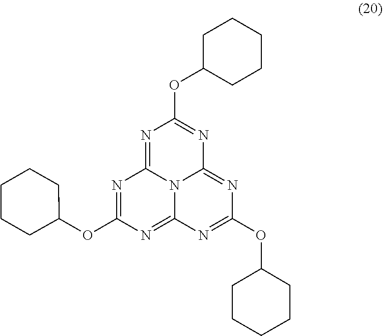 [SMILES: C1CCC(OC2=NC3=NC(OC4CCCCC4)=NC4=NC(OC5CCCCC5)=NC(=N2)N34)CC1]

Synthesis of the organic compound M was carried out as follows. Cyameluric acid chloride (138 mg, 0.5 mmol) and dimethylaminopyridine (220 mg, 1.8 mmol) were introduced in a flask, and cyclohexanol (3 mL) was added at room temperature in a nitrogen atmosphere. The temperature was raised to 60° C. after 5 minutes and to 70° C. after another hour, and stirring was carried out for 17 hours. After the temperature was returned to room temperature and water was added, extraction was carried out with chloroform, and an organic layer was dried with sodium sulfate and concentrated. Purification was carried out with a column (CHCl₃ 100%) to obtain an object. The resulting white solid organic compound M was 41 mg (0.088 mmol, 18%).

¹H NMR (600 MHz, CDCl₃) δ [ppm]=1.28-1.34 (m, 3H), 1.36-1.43 (m, 6H), 1.53-1.56 (m, 3H), 1.58-1.64 (m, 6H), 1.77-1.79 (m, 6H), 1.95-1.98 (m, 6H), 5.15-5.19 (m, 3H),

MS (ESI): 468.27 [calcd: 467.26]

(Luminescence Property of Organic Compound C)

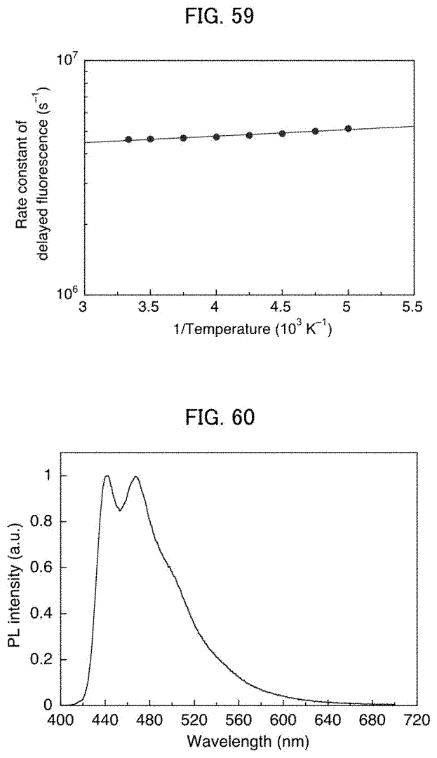

The organic compound C exhibited blue luminescence with a maximum emission wavelength of 449 nm in the toluene solution (see FIG. 57). A luminescent quantum yield in the toluene solution of the organic compound C was as high as 74%, and a luminescence lifetime τ indicated was short, i.e., 214 ns. From the temperature dependence of the luminescence lifetime τ, the energy gap ΔE_{ST} was estimated to be −6 meV and the radiative decay rate constant kᵣ was estimated to be 1.1×10⁷s⁻¹ (see FIG. 58 and FIG. 59).

(Luminescence Property of Organic Compound D)

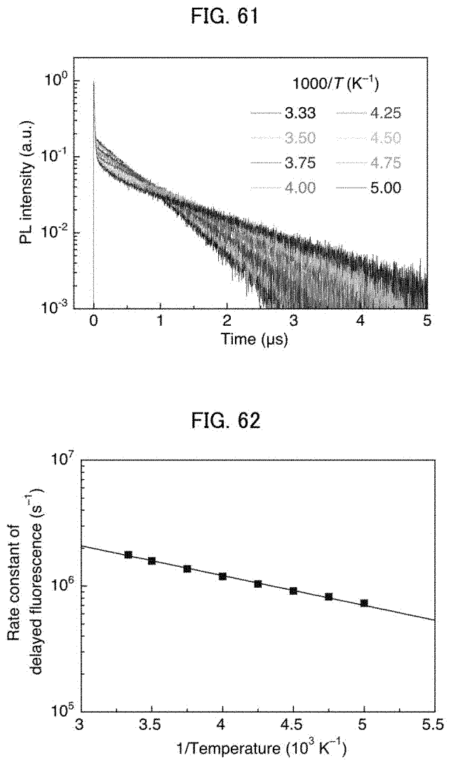

The organic compound D exhibited blue luminescence with a maximum emission wavelength of 442 nm in the toluene solution (see FIG. 60). A luminescent quantum yield in the toluene solution of the organic compound D was as high as 67%, and a luminescence lifetime τ indicated was short, i.e., 565 ns. From the temperature dependence of the luminescence lifetime τ, the energy gap ΔE_{ST} was estimated to be 47 meV and the radiative decay rate constant kᵣ was estimated to be 3.2×10⁷s⁻¹ (see FIG. 61 and FIG. 62).

(Luminescence Property of Organic Compound E)

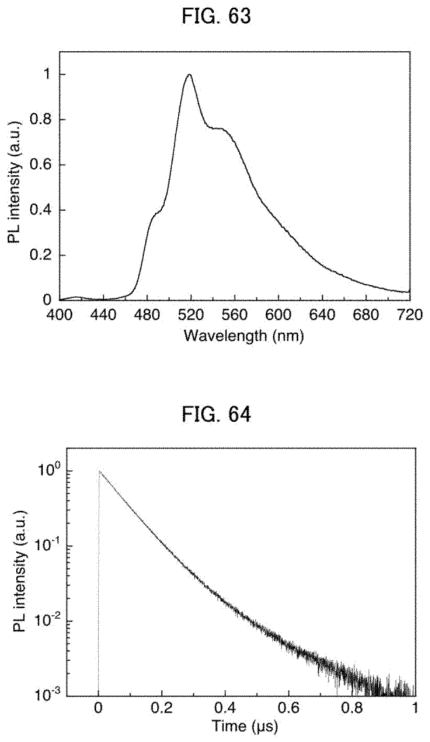

The organic compound E exhibited green luminescence with a maximum emission wavelength of 518 nm in the toluene solution (see FIG. 63). A luminescent quantum yield in the toluene solution of the organic compound E was 12%. No delayed fluorescence was observed from transient luminescent decay measurement, and only fluorescence with a luminescence lifetime τ of 90 ns was indicated (see FIG. 64).

(Luminescence Properties of Organic Compounds F Through M)

The luminescence properties of the organic compounds F, G, H, I, J, K, L, and M are indicated in Table 3. Table 3 also indicates the luminescence properties of the above described organic compounds C, D, and E.

As indicated in Table 3, the organic compounds F and G in the toluene solutions exhibited a negative energy gap ΔE_{ST}, as with the organic compound C. The organic compound H exhibited a positive energy gap ΔE_{ST}, as with the organic compound D. The organic compounds I, J, K, L, and M exhibited no delayed fluorescence, as with the organic compound E.

TABLE 3

| | | Luminescent | | | |
|---|---|---|---|---|---|
| | Maximum emission | quantum yield | τ | ΔE_{ST} | kᵣ |
| | wavelength (nm) | (%) | (ns) | (meV) | (s⁻¹) |
| Organic | 449 | 74 | 214 | −6 | 1.1 × 10⁷ s⁻¹ |
| Compound C | | | | | |
| Organic | 442 | 67 | 565 | 47 | 3.2 × 10⁷ s⁻¹ |
| Compound D | | | | | |
| Organic | 518 | 12 | —^{a} | —^{a} | —^{a} |
| Compound E | | | | | |
| Organic | 454 | 42 | 288 | −3 | 9.2 × 10⁶ s⁻¹ |
| Compound F | | | | | |
| Organic | 453 | 42 | 246 | −6 | 9.5 × 10⁶ s⁻¹ |
| Compound G | | | | | |
| Organic | 445 | 75 | 616 | 37 | 2.0 × 10⁷ s⁻¹ |
| Compound H | | | | | |
| Organic | 495 | 32 | —^{a} | —^{a} | —^{a} |
| Compound I | | | | | |
| Organic | 427 | 25 | —^{a} | —^{a} | —^{a} |
| Compound J | | | | | |
| Organic | 383 | —^{b} | —^{a} | —^{a} | —^{a} |
| Compound K | | | | | |
| Organic | 393 | —^{b} | —^{a} | —^{a} | —^{a} |
| Compound L | | | | | |
| Organic | 408 | —^{b} | —^{a} | —^{a} | —^{a} |
| Compound M | | | | | |

^{a}Unmeasurable because no delayed fluorescence was indicated
^{b}Unmeasurable because sufficient light emission intensity could not be obtained

<Evaluation of Organic Light Emitting Device>

A glass substrate provided with indium tin oxide (ITO) having a film thickness of 130 nm was ultrasonically cleaned in order of a neutral detergent, ultrapure water, acetone, and 2-propanol, then boiled in 2-propanol, and then UV ozone treatment was carried out for 30 minutes. This ITO-provided glass substrate was spin-coated with a dispersion liquid of poly(3,4-ethylenedioxythiophene):polystyrene sulfonate (PEDOT:PSS) (manufactured by Hereaeus, Clevious (registered trademark) CH8000) which had been diluted to 60% with ultrapure water in air and then dried at 200° C. for 10 minutes. Thus, a PEDOT:PSS film having a film thickness of 30 nm was formed. After that, by vacuum deposition, a molybdenum trioxide (MoO₃) film having a film thickness of 5 nm, a 4,4″-bis(triphenylsilanyl)-(1,1′,4′,1″)-terphenyl (BST) film having a film thickness of 3 nm, a bis(4-(dibenzo[b,d]furan-4-yl)phenyl)diphenylsilane (DBFSiDBF) film having a film thickness of 10 nm, a mixed film of 2,8-bis(diphenylphosphoryl)dibenzo[b,d]furan (PPF) and the organic compound C (10 wt %) having a film thickness of 15 nm, a PPF film having a film thickness of nm, a tris(8-hydroxyquinolinato)aluminium (Alq3) film having a film thickness of 40 nm, an (8-hydroxyquinolinato)lithium (Liq) film having a film thickness of 1 nm, and an aluminum film having a film thickness of 100 nm were formed to prepare an organic light emitting device. A luminescent area of the organic light emitting device is 2.0×2.0 mm². Structural formulae of PEDOT, PSS, BST, DBFSiDBF, PPF, Alq3, and Liq are as follows.

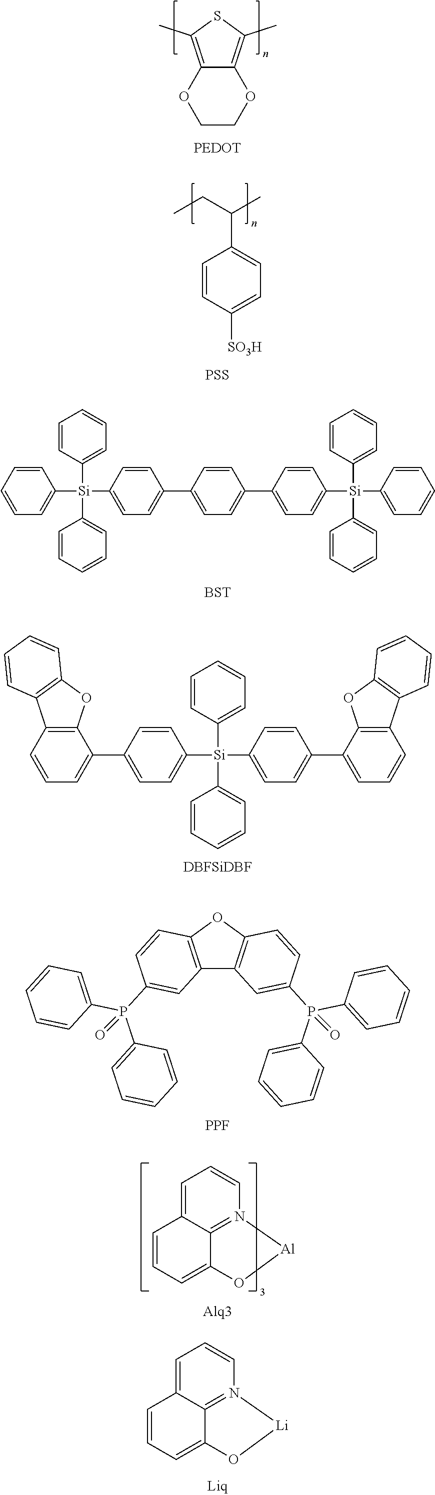

An organic light emitting device using 2,4,5,6-tetra(carbazol-9-yl)isophthalonitrile (4CzIPN) instead of the organic compound C were prepared in a similar manner.

The current density-voltage-luminance characteristics of the prepared organic light emitting device were measured using a Keithley 2400 source meter manufactured by Tektronix and a CS-200 luminance meter manufactured by Konica Minolta. The EL spectrum was measured using a PMA-11 multichannel spectroscope manufactured by Hamamatsu Photonics K.K. Transient luminescent decay was measured using an H7826 optical sensor manufactured by Hamamatsu Photonics K.K., a 33220A function generator manufactured by Agilent, and a DP03052 oscilloscope manufactured by Tektronix, while applying a pulse voltage (maximum of 8 V, minimum of −4 V) at a frequency of 1 KHz.

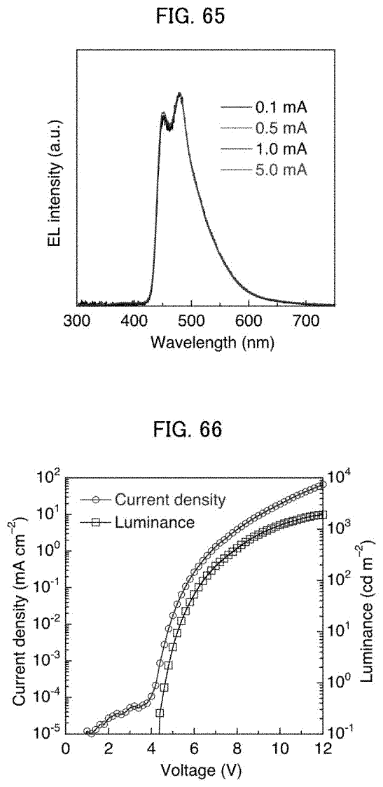

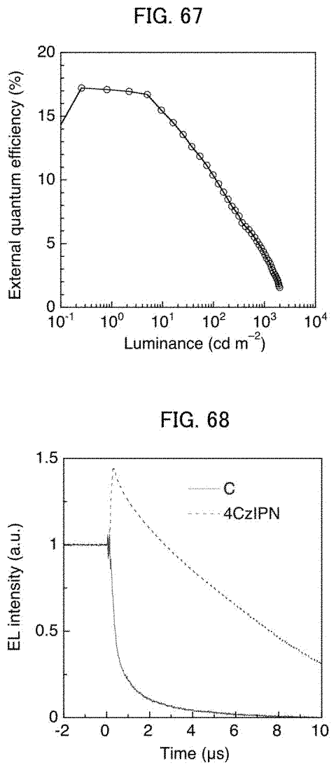

The organic light emitting device using the organic compound C exhibited blue luminescence from the organic compound C at an electric current of 0.1 mA to 5.0 mA (see FIG. 65). This organic light emitting device also exhibited good current density-voltage-luminance characteristics without leakage current or the like (see FIG. 66). In this organic light emitting device, the maximum external quantum efficiency of the organic compound C reached 17% (see FIG. 67). These results have indicated that the organic compound C can convert a triplet exciton into a singlet exciton and can be used as an organic light emitting device. In addition, as compared with 4CzIPN, which is a common TADF material, the organic compound C in this organic light emitting device exhibited faster transient luminescent decay (see FIG. 68). This is because the triplet exciton is rapidly converted to a singlet exciton and can be used as luminescence, resulting from the negative energy gap ΔE_{ST} in the organic compound C.

<Other Organic Compounds>

In addition to the above described organic compounds C through M, organic compounds p4-107, p4-4, p37-118, p139-139, p141-141, p142-142, p140-140, p162-162, and p65-166 were synthesized.

The organic compound p4-107 is represented by Formula (21) below.

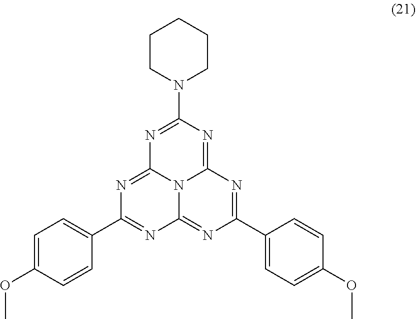 [SMILES: COc1ccc(C2=NC3=NC(c4ccc(OC)cc4)=NC4=NC(N5CCCCC5)=NC(=N2)N34)cc1]

Synthesis of the organic compound p4-107 was carried out as follows. Cyameluric acid chloride (70 mg, 0.3 mmol) was dissolved in dichloromethane (3 mL), and aluminium chloride (130 mg, 1.0 mmol) and methoxybenzene (80 mL, 0.8 mmol) were added at 0° C. After stirring for 15 minutes, the temperature was raised to room temperature, and stirring was carried out for 18 hours. An excessive amount of piperidine (1.0 mL) was added to the reaction liquid and diluted with water and dichloromethane after 30 minutes. An organic layer was separated, and dried with sodium sulfate. Then, concentration and column purification were carried out (AcOEt:CH₂C12=1:50-1:6) to obtain an organic compound p4-107. The resulting yellow solid organic compound p4-107 was 5.4 mg (0.012 mmol, 4.6%).

¹H NMR (600 MHz, CDCl₃) δ [ppm]=1.68-1.72 (m, 6H), 2.44 (s, 6H), 3.99 (br s, 4H), 7.29 (d, J=7.8 Hz, 4H), 8.44 (d, J=7.8 Hz, 4H)

MS (MALDI-TOF): 469.61 [calcd: 468.20]

The organic compound p4-4 is represented by Formula (22) below.

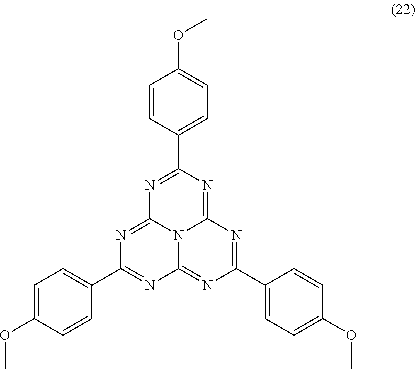 [SMILES: COc1ccc(C2=NC3=NC(c4ccc(OC)cc4)=NC4=NC(c5ccc(OC)cc5)=NC(=N2)N34)cc1]

Synthesis of the organic compound p4-4 was carried out as follows. Cyameluric acid chloride (100 mg, 0.36 mmol) was dissolved in dichloromethane (3 mL), and methoxybenzene (196 mL, 1.8 mmol) and aluminium chloride (173 mg, 1.3 mmol) were added at 0° C. 5 minutes later, the temperature was raised to room temperature, and stirring was carried out for 24 hours. After addition of water, an organic layer was separated and dried with sodium sulfate. After concentration, purification was carried out in a column (AcOEt:CHC₁₃=0:100-1:20) to obtain an object. The resulting yellow solid organic compound p4-4 was 34.5 mg (0.071 mmol, 19.8%).

¹H NMR (600 MHz, CDCl₃) δ [ppm]=3.92 (s, 9H), 6.99 (d, J=9 Hz, 6H), 8.57 (d, J=9 Hz, 6H)

MS (MALDI-TOF): 492.65 [calcd: 491.17]

The organic compound p37-118 is represented by Formula (23) below.

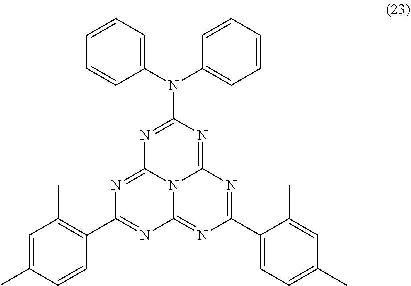

Synthesis of the organic compound p37-118 was carried out as follows. Cyameluric acid (623 mg, 2.26 mmol) was dissolved in m-xylene (20 mL) and diphenylamine (420 mg, 2.49 mmol) was added at room temperature. After stirring was carried out for 2.5 hours, the temperature was raised to 50° C., and stirring was carried out for another 2 hours. The resulting mixture was cooled to 0° C., aluminium chloride (904 mg, 6.8 mmol) was added, the resulting mixture was stirred at room temperature for 17 hours, and then water was added. Subsequently, 30 minutes later, chloroform was added, an organic layer was separated, and dried with sodium sulfate. Then, concentration and column purification were carried out (CHCl₃ 100%) to obtain an object. The resulting yellow solid organic compound p37-118 was 318 mg (0.58 mmol, 25.6%).

1H NMR (600 MHz, CDCl₃) δ [ppm]=2.34 (s, 6H), 2.63 (s, 6H), 7.03 (br s, 4H), 7.28-7.31 (m, 6H), 7.38 (t, J=7.8 Hz, 4H), 7.97 (d, J=8.4 Hz, 2H)

MS (MALDI-TOF): 549.94 [calcd: 548.24]

The organic compound p139-139 is represented by Formula (24) below.

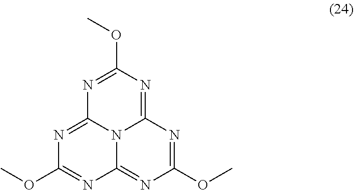

Synthesis of the organic compound p139-139 was carried out as follows. Cyameluric acid chloride (138 mg, 0.5 mmol) was dissolved in tetrahydrofuran (2 mL), and methanol (2 mL) and N,N-diisopropylethylamine (425 mL, 2.5 mmol) were added at room temperature in a nitrogen atmosphere. 20 minutes later, the temperature was raised to 60° C., and stirring was carried out for 24 hours. The temperature was returned to room temperature and water was added, and then the deposit was filtered and vacuum dried. The dried matter was dissolved in chloroform, filtered with silica gel, and cleaned with chloroform to obtain an object. The resulting white solid organic compound p139-139 was 35 mg (0.133 mmol, 27%).

¹H NMR (600 MHz, CDCl₃) δ [ppm]=4.10 (s, 9H)

MS (MALDI-TOF): 264.30 [calcd: 263.08]

The organic compound p141-141 is represented by Formula (25) below.

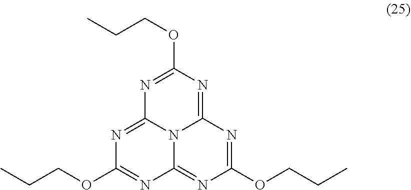 [SMILES: CCCOC1=NC2=NC(OCCC)=NC3=NC(OCCC)=NC(=N1)N23]

Synthesis of the organic compound p141-141 was carried out as follows. An intermediate 14 (228 mg, 0.5 mmol) represented by Formula (26) below was dissolved in 1-propanol (3 mL) and 2,4,6-trimethylpyridine (217 mL, 1.65 mmol) was added at room temperature in an argon atmosphere. After stirring for 10 minutes, the temperature was raised to 90° C., and stirring was carried out for 3 hours. After the temperature was returned to room temperature and water was added, extraction was carried out with chloroform, and then an organic layer was dried with sodium sulfate and concentrated. Purification was carried out with a column (AcOEt:CHC₁₃=5:95-15:85) to obtain an object. The resulting white solid organic compound p141-141 was 107 mg (0.31 mmol, 62%).

1H NMR (600 MHz, CDCl₃) δ [ppm]=1.00 (t, 9H), 1.80 (s, 6H), 4.43 (t, 6H)

MS (MALDI-TOF): 348.50 [calcd: 347.17]

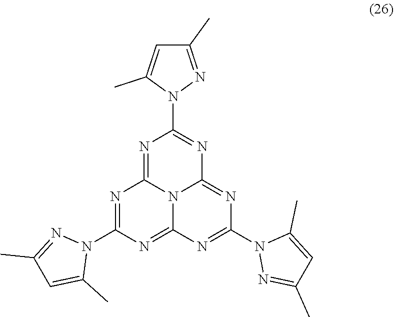 [SMILES: Cc1cc(C)n(C2=NC3=NC(n4nc(C)cc4C)=NC4=NC(n5nc(C)cc5C)=NC(=N2)N34)n1]

The synthesis of the intermediate 14 was carried out as follows. Cyameluric acid chloride (314 mg, 1.1 mmol) was dissolved in toluene (5 mL) and 3,5-dimethylpyrazole (362 mg, 3.8 mmol) and N,N-diisopropylethylamine (969 mL, 5.7 mmol) were added at room temperature in an argon atmosphere. The temperature was raised to 70° C. after 40 minutes and to 90° C. after another 20 minutes, and then stirring was carried out for 2 hours. After the temperature was returned to room temperature and water was added, extraction was carried out with chloroform, and then an organic layer was dried with sodium sulfate and concentrated. Purification was carried out with a column (MeOH:CHCl₃=1:99-10:90) to obtain an object. The resulting pale yellow solid intermediate 14 was 490 mg (1.08 mmol, 94%).

¹H NMR (600 MHz, CDCl₃) δ [ppm]=2.34 (s, 9H), 2.76 (s, 9H), 6.11 (s, 3H)

MS (MALDI-TOF): 456.54 [calcd: 455.20]

The organic compound p142-142 is represented by Formula (27) below.

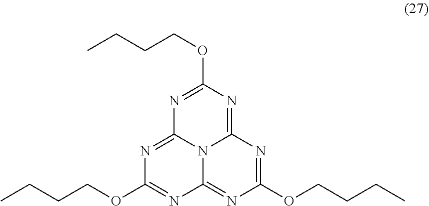 [SMILES: CCCCOC1=NC2=NC(OCCCC)=NC3=NC(OCCCC)=NC(=N1)N23]

Synthesis of the organic compound p142-142 was carried out as follows. Cyameluric acid chloride (358 mg, 1.3 mmol) was dissolved in tetrahydrofuran (3 mL), and 1-butanol (3 mL) and N,N-diisopropylethylamine (1.1 mL, 6.5 mmol) were added at room temperature in an argon atmosphere. After the addition, temperature was raised to 70° C. and to 90° C. after 2 hours, and stirring was carried out for 1.5 hours. After the temperature was returned to room temperature and water was added, extraction was carried out with chloroform, and then an organic layer was dried with sodium sulfate and concentrated. Purification was carried out with a column (AcOEt:CH₂C12=1:99-10:90) to obtain an object. The resulting white solid organic compound p142-142 was 374 mg (0.96 mmol, 74%).

¹H NMR (600 MHz, CDCl₃) δ [ppm]=0.95 (t, 9H), 1.45 (tq, 6H), 1.76 (tt, 6H), 4.47 (t, 6H), MS (MALDI-TOF): 390.64 [calcd: 389.22]

The organic compound p140-140 is represented by Formula (28) below.

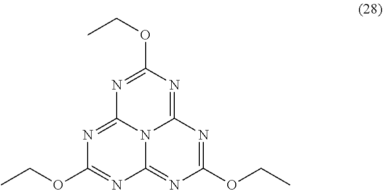 [SMILES: CCOC1=NC2=NC(OCC)=NC3=NC(OCC)=NC(=N1)N23]

Synthesis of the organic compound p140-140 was carried out as follows. Cyameluric acid chloride (456 mg, 1.65 mmol) was dissolved in tetrahydrofuran (5 mL), and ethanol (5 mL) and N,N-diisopropylethylamine (1.4 mL, 8.3 mmol) were added at room temperature in an argon atmosphere. 2 hours later, the temperature was raised to 80° C., and stirring was carried out for 17 hours. After the temperature was returned to room temperature and water was added, extraction was carried out with dichloromethane, and then an organic layer was dried with sodium sulfate and concentrated. Purification was carried out with a column (AcOEt:CH₂C12=5:95-15:85) to obtain an object. The resulting white solid organic compound p140-140 was 172 mg (0.56 mmol, 34%).

¹H NMR (600 MHz, CDCl₃) δ [ppm]=1.41 (t, 9H), 4.53 (q, 6H)

MS (MALDI-TOF): 306.47 [calcd: 305.12]

The organic compound p162-162 is represented by Formula (29) below.

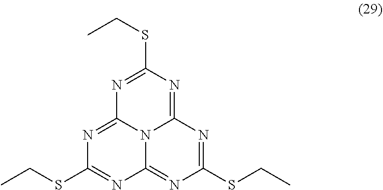 [SMILES: CCSC1=NC2=NC(SCC)=NC3=NC(SCC)=NC(=N1)N23]

Synthesis of the organic compound p162-162 was carried out as follows. Cyameluric acid chloride (221 mg, 0.8 mmol) was dissolved in toluene (5 mL), and ethanethiol (592 mg, 4.0 mmol) and N,N-diisopropylethylamine (680 mL, 5.7 mmol) were added at room temperature in an argon atmosphere. 30 minutes later, the temperature was raised to 35° C., and stirring was carried out for 17 hours. After the temperature was returned to room temperature and water was added, extraction was carried out with chloroform, and then an organic layer was dried with sodium sulfate and concentrated. Purification was carried out with a column (AcOEt:CH₂C12=0:100-5:95) to obtain an object. The resulting white solid organic compound p162-162 was 234 mg (0.66 mmol, 83%).

¹H NMR (600 MHz, CDCl₃) δ [ppm]=1.37 (t, 9H), 3.16 (q, 6H)

MS (MALDI-TOF): 354.43 [calcd: 353.06]

The organic compound p65-166 is represented by Formula (30) below.

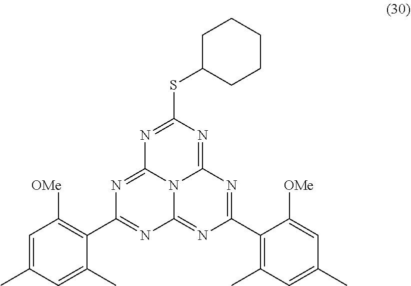

Synthesis of the organic compound p65-166 was carried out as follows. To a dichloromethane (11.8 mL) solution of the intermediate 13 (608 μL, 4.3 mmol), aluminum chloride (616 mg, 4.6 mmol) was added at room temperature and stirred for 40 minutes. A dichloromethane (12 mL) solution of a compound 2 was slowly added and stirred at room temperature for 20.5 hours. A 1-M sodium hydroxide aqueous solution (16 mL) was added at 0° C., stirred at room temperature for 4 hours, and then filtered using cerite. A 20% sodium chloride aqueous solution was added to the reaction liquid, and then an organic layer was separated and dried with anhydrous sodium sulfate. Then concentration and column purification (CH₂C12 CH₂C12:MeOH=9:1) were carried out to obtain a crude body (117 mg). The crude body was purified (SunFire, Hexane/EtOAc=82:18) in a preparative column to obtain an organic compound p65-166 as a third peak. The resulting yellow solid organic compound p65-166 was 13.8 mg (0.025 mmol, 2.3%).

1H NMR (600 MHz, CDCl₃) δ [ppm]=0.80-0.92 (br, 2H), 1.20-1.38 (br, 2H), 1.38-1.50 (m, 2H), 1.66-1.76 (br, 2H), 2.00-2.08 (br, 2H), 2.31 (s, 6H), 2.32 (s, 6H), 3.78 (s, 6H), 3.89-3.98 (br, 1H), 6.57 (s, 2H), 6.63 (s, 2H) The crude body was purified (SunFire, Hexane/EtOAc=82:18) in a preparative column to obtain an organic compound represented by Formula (31) below as a second peak. The resulting yellow solid organic compound was 22.4 mg (0.040 mmol, 3.8%).

1H NMR (600 MHz, CDCl₃) δ [ppm]=0.80-0.92 (br, 2H), 1.20-1.38 (br, 2H), 1.38-1.50 (m, 2H), 1.68-1.77 (br, 2H), 2.00-2.10 (br, 2H), 2.31 (s, 3H), 2.32 (s, 3H), 2.41 (s, 6H), 3.78 (s, 3H), 3.79 (s, 3H), 3.86-3.98 (br, 1H), 6.58 (s, 1H), 6.59 (s, 2H), 6.64 (s, 1H)

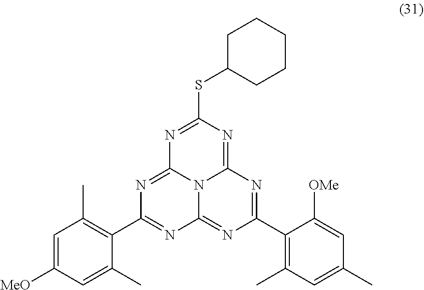 [SMILES: COc1cc(C)c(C2=NC3=NC(c4c(C)cc(C)cc4OC)=NC4=NC(SC5CCCCC5)=NC(=N2)N43)c(C)c1]

[Additional Remarks]

The present invention is not limited to the embodiments, but can be altered by a skilled person in the art within the scope of the claims. The present invention also encompasses, in its technical scope, any embodiment derived by combining technical means disclosed in differing embodiments.

### Industrial Applicability

The present invention can be utilized as a luminescent material.

1. An organic compound having a lone electron-pair and a π electron orbit, wherein an energy gap ΔE_{ST} obtained by subtracting an energy level E_{T1} of a lowest triplet excitation state T₁ from an energy level E_{S1} of a lowest singlet excitation mode S₁ is −0.20 eV≤ΔE_{ST}<0.0090 eV.

2. The organic compound as set forth in claim 1, wherein a radiative decay rate constant kᵣ is 1.0×10⁶s⁻¹<kᵣ.

3. The organic compound as set forth in claim 1, wherein an oscillator strength f is 0.0050<f.

4. The organic compound as set forth in claim 1, wherein said organic compound is a heptazine derivative that is represented by Formula (1) below and that has three arbitrary substituents R1, R2, and R3 which are independent of each other.

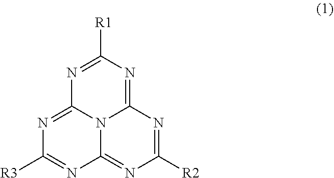

5. The organic compound as set forth in claim 4, wherein the substituents R1, R2, and R3 are constituted by two types of substituents.

6. The organic compound as set forth in claim 4, wherein the substituents R1, R2, and R3 are constituted by three different types of substituents.

7. The organic compound as set forth in claim 4, wherein the substituents R1, R2, and R3 are constituted by one type of substituent.

8. An organic compound having a lone electron-pair and a π electron orbit, wherein:
said organic compound is a heptazine derivative that is represented by Formula (1) below and that has three arbitrary substituents R1, R2, and R3 which are independent of each other; and
the substituents R1, R2, and R3 are constituted by two types or three types of substituents.

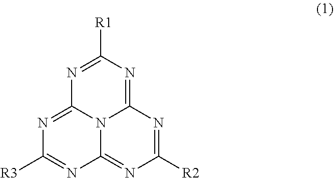

9. An organic light emitting device comprising an organic compound recited in claim 1.

10. The organic light emitting device as set forth in claim 9, further comprising a luminescent layer that contains a host compound and the organic compound which functions as a dopant compound.

11. An organic light emitting device, comprising a luminescent layer that contains a dopant compound and a host compound,
the host compound being an organic compound which has a lone electron-pair and a π electron orbit and in which an energy gap ΔE_{ST} obtained by subtracting an energy level E_{T1} of a lowest triplet excitation state T₁ from an energy level E_{S1} of a lowest singlet excitation mode S₁ is negative or 0 eV≤ΔE_{ST}<0.0090 eV.

12. An organic light emitting device comprising a luminescent layer containing a dopant compound and a host compound,
the host compound being a heptazine derivative that has a lone electron-pair and a 7E electron orbit, that is represented by Formula (1) below, and that includes an arbitrary substituent R1. [chemical structure]
